(19) United States
(12) Patent Application Publication
(10) Pub. No.: US 2004/0057926 A1
(43) Pub. Date: Mar. 25, 2004

(54) Modulation of the immune response through the manipulation of arginine levels

(75) Inventors: Augusto C. Ochoa, New Orleans, LA (US); Juan B. Ochoa, Pittsburgh, PA (US); Mircea Popescu, Plainsboro, NJ (US); Arnold H. Zea, Metairie, LA (US); Paulo C. Rodriguez, Metairie, LA (US)

Correspondence Address: FOLEY AND LARDNER, SUITE 500, 3000 K STREET NW, WASHINGTON, DC, 20007, US

(73) Assignee: LSU Medical Center

(21) Appl. No.: 10/386,131

(22) Filed: Mar. 12, 2003

Related U.S. Application Data
(60) Provisional application No. 60/363,366, filed on Mar. 12, 2002.

(51) Int. Cl. A61K 38/19; A61K 31/69; A61K 31/198

(52) U.S. Cl. 424/85.1; 514/64; 514/561; 514/564; 514/565

(57) ABSTRACT

The present invention provides methods and compositions for modulating an immune response by controlling the level of arginase available to a cell, tissue or system. An immune response can be enhanced or depressed by altering the amount of arginine available to a cell, tissue or system through the manipulation of localized or systemic arginine levels using substances which provide arginine to the body and enzymes which break down arginine, such as arginase and nitric oxide synthase. Increasing or decreasing an immune response according to the present invention provides therapeutic treatments for a variety of conditions and diseases. The present invention also provides clinical methods and kits which can measure the strength or resistance to an immune response in a cell, tissue or system based upon the amount of available arginine and enzymes which break down arginine.

### CLAIM OF PRIORITY

This application claims priority from U.S. Provisional Patent Application No. 60/363,366, filed Mar. 12, 2002.

### STATEMENT REGARDING FEDERALLY SPONSORED RESEARCH

This work was supported in part by grants RO1-CA82689, RO1-CA88885, NCI-PO1 CA028842, KO8 GN 0646 and X08-GM00676-01 from the National Cancer Institute and the National Institutes of Health—(NIH-NCI), Bethesda, Md. The Government has certain rights in this invention.

### FIELD OF THE INVENTION

The present invention relates generally to modulating or measuring the strength of an immune response. More particularly, this inventions relates to modulating or measuring an immune response through the manipulation or measurement of arginine and enzyme levels which degrade arginine.

### BACKGROUND OF THE INVENTION

L-arginine is used as the substrate to produce either nitric oxide (NO) or urea and ornithine. This process is carefully controlled by the enzymes nitric oxide synthase and arginase (I and II) in macrophages and possibly other cells. In the normal response, the damage to any tissue, be it inflicted by an outside process (trauma, burns, infection etc.) or an internal process (tumor, autoimmune responses, etc), initiates an immune response that tries to achieve two results:

1) Protect the tissues from bacterial invasion and eliminate any dead tissue.

2) Initiate the healing process of the damaged tissues.

Nitric oxide is preferentially produced by macrophages when it needs to destroy bacteria or eliminate dead tissue. For this the macrophage metabolizes L-arginine through the Nitric Oxide Synthase pathway resulting in the production of NO and nitrites. Once this process has been completed and the macrophage shifts the metabolism of L-arginine to produce urea and ornithine through the arginase pathway (arginase I). There are two types of arginase—arginase I which is produced by the liver and macrophages and arginase II which is non-hepatic. Arginase I is inducible in macrophages by cytokines while arginase II is constitutively expressed. Ornithine serves as the basis for the production of proline and polyamines needed for cell proliferation and stimulates the production of collagen by the fibroblasts. This phenomenon leads to the eventual healing (scar formation) of the damaged tissue.

In disease however, normal immune function is disrupted leading to an excessive production of arginase I and the depletion of arginine, which results in an impaired immune function. Most of these altered events are seen in diseases where there is “chronic inflammation.” Examples of these diseases include tumors, lupus erythematosus, rheumatoid arthritis, chronic infectious processes (tuberculosis, HIV, leprosy, chronic active hepatitis) and pulmonary diseases such as emphysema and chronic obstructive pulmonary disease (COPD). Furthermore certain instances of massive tissue damage (as in extensive trauma and burns) can lead to a disruption of this balanced management of L-arginine. In all these diseases which result in an alteration in the management of L-arginine levels can lead to the disruption of the immune response (with all of its known consequences) or can lead to tissue damage by excessive scarring. In the clinical setting the practical consequences of this is as follows:

Exacerbation of arginase 1 production (as in uncontrolled autoimmune disease, chronic infections and cancer) can lead to two problems, a severe decrease in the immune response and the development of damaging scar tissue. Patients with trauma, cancer and systemic lupus have a severely impaired immune response. Likewise, the continued inflammatory process of autoimmunity leads to the development of damaging scar tissue (fibrosis), as in rheumatoid arthritis.

Thus there remains a need to control immune responses for therapeutic benefit.

### SUMMARY OF THE INVENTION

According to the present invention, the level of arginine in a cell, tissue or bodily system is modulated in order to regulate an immune response. Preferred non-limiting example of cells, tissues and systems which can have their arginine levels modulated according to the present invention include lymphocytes (especially T-lymphocytes), monocytes, macrophages, dendritic cells, cancer cells (tumors), tissues undergoing or which have recently undergone trauma, bone marrow, organs, connective tissues, cartilage, circulatory system, arginine producing cells, arginase producing cells and the reticuloendothelial system.

One embodiment of invention provides a method of treating an arginase I mediated immune suppression or depleted arginine levels in a mammal in need thereof that includes:

Administering an effective amount of an arginase I inhibitor, an inhibitor of a cationic amino acid transporter Y+ receptor, such as a CAT-2B cationic amino acid transporter, or a liposomal formulation of arginine, an arginine provider, an arginase inhibitor or CAT-2B inhibitor to a mammal in need thereof wherein an immune response in the mammal is increased, generally compared to the immune response in the absence of treatment with the inhibitor of arginase I or the inhibitor of the CAT-2B cationic amino acid transporter. In some embodiments the arginase inhibitor is administered alone. In other embodiments, the cationic amino acid transporter Y+ receptor is administered alone. In some embodiments both the arginase I inhibitor and the inhibitor of a cationic amino acid transporter Y+ receptor are administered in a combination therapy. When both the arginase inhibitor and the cationic amino acid transporter Y+ receptor are administered in a combination therapy, these inhibitors can be administered in any given order. For example the arginase inhibitor can be administered first or second, although in some embodiments they are administered in close proximity to one another. The inhibitors can also be administered simultaneously. In some embodiments, the inhibitors can be provided together in a single pharmaceutical formulation or in separate formulations as desired. Thus, the present invention also provides these compositions comprising these formulations. In some of these formulations, either or both of the inhibitors can be encapsulated in liposomes and administered as a liposomal formulation. These liposomal formulation can be targeted to specific tissues or arginase producing tissues and cells as described herein, such as cancers, infectious agents, macrophages, dendritic cells or T-cells. The inhibitors, either alone or in combination, can also be administered with arginine or an arginine provider, which can be encapsulated in a liposome. In some of the treatments and administration methods described herein the inhibitors or arginase or cationic amino acid transporter Y+ receptor or the arginine providers can be administered multiple times to the subject.

In the present methods, increased immune responses can be measured by any methods or assays known to those skilled in the art. For example, in some embodiments, the level of immune response can be determined by measuring re-expression of zeta chain of the TCR complex after antigen stimulation. In other embodiments, immune response cn be measured by T-cell response against an antigen or by T cell proliferation. In other embodiments, a measure of immune response can be determined by arginine levels, either systemic or localized arginine levels. In another embodiment the efficacy of the treatment can also be determined by measuring the arginase levels and/or activity of the arginase producing cells, e.g. tumor cells, infectious organisms, or macrophages, either systemically or locally to the cells themselves. The arginase levels can then be correlated with the immune response as described herein. Additional methods for determining the efficacy of the treatment are described herein. Methods for performing these measurements are also described in the examples.

In another embodiment, a method of treating an arginase mediated immune suppression resulting from a bacterial or viral infection in a mammal in need thereof is provided. This embodiment involves at least administering an effective amount of an inhibitor of arginase, an inhibitor of a CAT-2B cationic amino acid transporter or a liposomal formulation of arginine or an arginine provider to a mammal suffering having a bacterial or viral infection in need thereof. Generally, in this method an immune response in the mammal is increased, such as by comparison to the immune response in the absence of treatment with the inhibitor of arginase I or the inhibitor of the CAT-2B cationic amino acid transporter. In some of these methods the infection is not a result of leishmaniasis.

In the above embodiments, the inhibitor of arginase I or the inhibitor of the CAT-2B cationic amino acid transporter can be a competitive inhibitor. In some of these embodiments, the mammal is a human. In other embodiments, the arginase I mediated immune suppression is caused by a chronic infectious disease, autoimmune disease, trauma, leprosy, tuberculosis, liver transplantation, infectious microorganisms such as bacteria, viruses or parasites or a cancer. In some embodiments when the disease being treated is cancer the cancer is other than gastric cancer or breast cancer. In yet other embodiments, the inhibitor of arginase I or the inhibitor of the CAT-2B cationic amino acid transporter is cycloheximide, NOHA, nor-NOHA, ornithine, lysine, norvaline, adrenergic blocking agents, propanolol, a cytokine, L-mono-methyl-L-arginine (NMMA), a boronic acid based compound, 2(S)-amino-6boronohexanoic acid (ABH) and S-(2-boronoethyl)-L-cysteine (BEC), and combinations thereof. In some of the above embodiments, the immune response increased in the mammal comprises increasing stimulated T-cell proliferation, T-cell function or both. In some of the embodiments, the T-cell is a stimulated T-cell. In still other embodiments, the immune response increased in the mammal is a systemic immune response. In some of the above methods the arginase inhibitor preferentially inhibits arginase I compared to arginase II. In some of the above embodiments, the inhibitor of a CAT-2B cationic amino acid transporter or the liposomal formulation of arginine or an arginine provider is administered in amount such that the arginine level available to the T-cells of the subject is about 40 μM, 80 μM, 120 μM or greater.

Another embodiment of the present invention provides a method of therapeutically suppressing an immune response in a mammal. Generally, this method includes administering an effective amount of arginase I or a stimulator of arginase I to a mammal wherein an immune response in the mammal is suppressed, such as by comparison to the immune response in the absence of treatment with the stimulator of arginase I. In some embodiments, the stimulator of arginase I can increase the arginase I levels or increase the activity of the arginase I. In some of the above embodiments, the stimulator of arginase I is a Th2 cytokine, IL-4, IL-10, IL-13, 8-bromo-cAMP (Morris et al. Am. J. Physiol. November 1998;275(5 Pt 1):E740-7), 8-bromo-cAMP plus Lipopolysaccharide 8-bromo-cAMP and interferon-gamma or combinations thereof.

Another aspect of the present invention involves measuring the amount of arginine or arginase in a medium in order to determine the strength of a potential immune response or resistance to an immune response, and kits provided therefore.

As will be understood by the skilled artisan all of the embodiments and aspects of the methods disclosed herein can be suitably used with all other appropriate aspects and embodiments as disclosed herein to define the present invention.

### BRIEF DESCRIPTION OF THE DRAWINGS

The foregoing advantages and features of the invention will become apparent upon reference to the following detailed description and the accompanying drawings, of which:

FIG. 1 shows the difference between ζ chain expression in Jurkat cells cultured in media with and without arginine;

FIG. 2a shows the effect on ζ chain expression over time in Jurkat cells cultured in arginine-free media;

FIG. 2b shows the effect on CD3ε expression over time in Jurkat cells cultured in arginine-free media;

FIG. 2c shows the effect on TCRαβ expression over time in Jurkat cells cultured in arginine-free media;

FIGS. 3a, 3b and 3c show the effect on ζ chain expression over time in Jurkat cells cultured in glutamine-free media;

FIGS. 4a and b show the effect on ζ chain expression of Jurkat cells cultured in arginine-free media subsequently transferred to media containing arginine;

FIGS. 5a and b show the effect of arginine depletion on the re-expression of ζ chain and TCR in Jurkat cells after antigen stimulation; and

FIG. 6 shows a decrease in ζ chain expression over time in cells cultured in arginine free medium.

FIG. 7 shows the absence of L-arginine in tissue culture media induces a sustained decrease in CD3ζ. Mean intensity fluorescence for CD3ε (A) and CD3ζ (B) was measured in purified T-cells stimulated with anti-CD3 plus anti-CD28 in RPMI (-□-) or Arg-free-RPMI (-⋄-) or cultured without stimulation in RPMI (-◯-) or Arg-free-RPMI (-∇-). Data is of four different experiments and are presented as the mean±SEM. (C), T-cells stimulated and cultured in the presence (A+) or absence (A−) of L-arginine, were tested at different time points for the expression pf CD3ζ, CD3ε and GAPDH by Western blot.

FIG. 8 shows the addition of L-arginine to the culture media induces the recovery of CD3ζ. A. T-cells were stimulated and cultured in Arg-free-RPMI (-⋄-) for 24 h then L-arginine (1140 μM) was added to the media (-♦-) and the expression of CD3ζ was measured by flow cytometry at 24, 48 and 72 h. B. Normal T-cells were stimulated and cultured in the absence of L-arginine (-⋄-), L-glutamine (-Δ-), L-glysine (-∇-) or in RPMI (-□-). CD3ζ was measured by flow cytometry.

FIG. 9 shows T-cell proliferation is significantly decreased in T-cells cultured in Arg-free-RPMI after antigen stimulation. Unstimulated and stimulated T-cells were pulsed with [³H] thymidine. Data from three experiments are presented as a mean±SEM (represented by the error bars). *p<0.001; **p<0.002

FIG. 10 shows: A. Production of IL2, IFNγ, IL5 and IL10 in T-cells stimulated and cultured in RPMI and Arg-free-RPMI. Data from five experiments is presented as a mean±SEM. B. Ribonuclease protection assay (RPA) shows the mRNA expression for the different cytokines in T-cells stimulated and cultured for 24, 48 and 72 h in the presence or absence of L-arginine. *p<0.001

FIG. 11 shows the absence of L-arginine does not induce changes in CD3ζ mRNA levels and it is not due to protein degradation A. Effect of lysosomes (bafilomycin) and proteasomes (lactasystin) inhibitors in T-cells stimulated and cultured in RPMI or Arg-free-RPMI for 48 h. CD3ζ expression in T-cells was tested by flow cytometry. B. Stimulated T-cells were cultured in RPMI or Arg-free-RPMI for 1, 2, 24, 48 and 72 h. Northern blot analysis was performed for the detection of the expression of CD3ζ mRNA using 10 μg of total RNA. GAPDH was used as a house keeping gene. C. T-cells were cultured in RPMI or Arg-free-RPMI for 12, 24 and 48 h, then actinomicyn D (5 mg/ml) and total RNA was extracted at 2, 4 and 8 h, electrophoresed and hybridized with CD3ζ probe.

FIG. 12 depicts that metabolic labeling demonstrates a decreased CD3ζ synthesis T-cells stimulated and cultured in Arg-free-RPMI. T-cells were labeled with [³⁵S] methionine lysed and immunoprecipitated with anti-CD3 at 24, 48, 72 and 96 h. Cells labeled at time of harvesting (time 0) were used as a controls. To determine the specificity of the labeled proteins, irrelevant monoclonal antibodies (Isotype, mouse IgG₁ monoclonal antibody) were used for immunoprecipitation controls.

FIG. 13: PM stimulated with IL-4+IL-13 display an increased expression of ASE I and decrease the extra-cellular levels of L-Arg. (a) 2×10⁶ PM were stimulated with IL-4+IL-13 or IFN-γ for 24 hours. Cytoplasmic extracts were isolated and western blots were done for ASE I, ASE II, iNOS and GAPDH. (b) Nitrite levels were measured in the supernatants using the Griess reagent as an indirect measure of NO production. Bars represent the mean levels of nitrites in 3 different experiments±SD. (c) Cytoplasmic extracts from 2×10⁶ PM stimulated with IL-4+IL-13 were harvested at different time points and tested for ASE I, ASE II and GAPDH expression by western blot. (d) Supernatants from cultures of PM stimulated with IL-4+IL-13 or IFN-γ were tested for L-Arg concentration by HPLC at 3, 6, 12 and 24 hours after stimulation. Results show the mean±SD of 3 different experiments.

FIG. 14: Co-culture of Jurkat cells and T lymphocytes with IL-4+IL-13 stimulated PM results in a decreased CD3ζ expression. (a) Co-cultures of 2×10⁶ PM stimulated with IL-4+IL-13 and 1×10⁶ Jurkat cells were done in 0.4 μm pore transwells (Boyden chambers). Jurkat cells were on the top chamber, while activated PM were on the bottom chamber. Jurkat cells were harvested at 24 and 48 hours and tested for CD3ζ, CD3ε and GAPDH by western blot. (b) Co-culture of PM and Jurkat cells were done in transwells using different numbers of PM stimulated with IL-4+IL-13 or IFN-γ ranging from 0.25-2.0×10⁶ cells per well and 1×10⁶ Jurkat cells. All cells were cultured in RPMI containing 150 μM L-Arg. CD3ζ expression was measured by flow cytometry 24 hours later. (c) Co-cultures of 2×10⁶ PM and 1×10⁶ Jurkat cells were done as described previously. Jurkat cells were harvested and tested for CD3ζ expression by flow cytometry at different times in culture. (d) PM stimulated with IL-4+IL-13 or IFN-γ were co-cultured with 1×10⁶ T lymphocytes that were previously stimulated with cross-linked anti-CD3+anti-CD28. The expression of CD3ζ was measured by flow cytometry. FIG. 2B, C, D represent the mean±SD of CD3ζ expression in 3 different experiments.

FIG. 15: ASE I but not iNOS, ASE II or hydrogen peroxide induces a decreased expression of CD3ζ in Jurkat cells and normal T lymphocytes. (a) 2×10⁶ PM were stimulated with IL-4+IL-13 for 24 hours in RPMI containing 150 μL-Arg. Inhibitors NOHA (100 μM), Nor-NOHA (50 μM), L-NIL (5 μg/ml) and hydrogen peroxide scavenger catalase (200 U/ml) were added at time 0. A culture with excess L-Arg (2 mM) added at time 0 was also included. 1×10⁶ Jurkat cells were added onto 0.4μ transwells and CD3ζ was tested by flow cytometry after an additional 24 hours of culture. Bars represent the mean±SD CD3ζ expression in 3 different experiments. (b) PM stimulated with IL-4+IL-13 were co-cultured with stimulated T-cells in the presence of NOHA or Nor-NOHA. (c) Western blot for ASE I was done using cytoplasmic extracts from macrophages stimulated with IL-4+IL-13 or IFN-γ in the presence or absence of NOHA (100 μM) or Nor-NOHA (50 μM). (d) These cytoplasmic extracts were also tested for ASE activity by measuring L-ornithine production in the presence or absence of NOHA (100 μM) or Nor-NOHA (50 μM). All experiments were repeated at least 3 times. The data represent the mean±SD in 3 different experiments.

FIG. 16: Increased L-Arg uptake and CAT-2B expression in PM stimulated with IL-4+IL-13. (a) 1×10⁶ PM were stimulated with IL-4+IL-13 or IFN-γ and cultured in RPMI containing 150 μM L-Arg and 5 μCi of ³H-L-Arg. Cells were detached using Trypsin/EDTA and washed twice with D-PBS. ³H-L-Arg uptake was measured at 6, 12 and 24 hours. ***P<0.005. (b) 2×10⁶ PM were stimulated with IL-4+IL-13 or IFN-γ and RNA isolated at 3, 6, 12, 24 and 48 h. CAT-2B mRNA expression was measured by northern blot. (c) 2×10⁶ PM were stimulated with IL-4+IL-13 or IFN-γ for 24 hours in the presence of L-Arg analogues L-NMMA (1 mM), L-NNA (1 mM) and L-NAME (1 mM) in RPMI containing 150 μM L-Arg. 1×10⁶ Jurkat cells were added onto 0.4μ transwells, and CD3ζ was tested by flow cytometry after an additional 24 hours. (d) 2×10⁶ PM stimulated with IL-4+IL-13 were co-cultured with 1×10⁶ Jurkat cells in transwells. After 24 hours of co-culture, 2 mM exogenous L-Arg or 2 mM L-glutamine was added and CD3ζ was tested by flow cytometry 24 hours later. FIG. 4A, C, D represent the mean CD3ζ expression in 3 different experiments±SD.

FIG. 17: H. pylori sonicate impairs the proliferation of Jurkat cells and PBMC. Jurkat cells and PBMC (2×10⁵/well) stimulated with anti-CD3 plus anti-CD28 antibodies were cultured with increasing concentrations of H. pylori sonicate for 2 h. One μCi of ³H-thymidine was added for the last 20 h of culture. A. Proliferation of Jurkat cells and PBMC after 24 h and 48 h of culture, respectively. The results show the average±SEM of three experiments. B. Jurkat cells were cultured with (50 g/ml) or without the H. pylori sonicate for up to 96 h to study its effects on proliferation over time. The results show the average±SEM of two experiments.

FIG. 18: H. pylori sonicate does not alter the pattern of tyrosine phosphorylation in Jurkat cells. Jurkat cells were cultured in the presence of 50 μg/ml of H. pylori sonicate during 4 h and 8 h, washed and lysed. The proteins were separated by electrophoresis, transferred to membranes and immunobloted with anti-phosphotyrosine. The radiograph shows the pattern of phosphorylation in Jurkat cells cultured in the absence (−) or presence (+) of sonicate during the times shown. No significant differences in the phosphorylation pattern were observed.

FIG. 19: H. pylori sonicate reduces the expression of CD3ζ chain in Jurkat cells. Jurkat cells incubated with H. pylori sonicate for 24 h were stained for CD3ζ. Control cells were left untreated (labeled as 0). The mean±SEM of at least six experiments is shown. *p=0.0004 and **p<0.0001 as compared to cells without H. pylori sonicate.

FIG. 20: H. pylori proteins do not change CD3ζ expression in Jurkat cells. Jurkat cells were cultured for 24 h in the presence of purified H. pylori proteins CagA (2.5 μg/ml), VacA (4.0 μg/ml), UreA (2.0 μg/ml), UreB (2.0 μg/ml), H. pylori sonicate (50 μg/ml). Cells were stained for CD3ζ and mean fluorescence intensity (MFI) tested by flow cytometry. The mean±SEM of three experiments is shown. *p=0.01 as compared to non-stimulated Jurkat cells.

FIG. 21: Arginase inhibitor NOHA or excess L-arginine re-establishes CD3ζ expression and proliferation in Jurkat cells treated with H. pylori sonicate. Fifty micrograms of the H. pylori sonicate were pre-incubated overnight with either L-arginine or NOHA and the mixture added to the cells as described in Materials and Methods. A. Proliferation of Jurkat cells treated with H. pylori sonicate in the presence of L-arginine (2 mM) or NOHA (10 μg/ml). The mean±SEM of two experiments is shown. *p=0.03 compared to unstimulated Jurkat cells; **p=0.0003, ***p=0.007 as compared to Jurkat cells treated with 50 μg/ml of H. pylori sonicate. B. Expression of CD3ζ in Jurkat cells treated with the H. pylori sonicate in the presence of NOHA. The mean±SEM of the mean fluorescence intensity for CD3ζ of three different experiments is shown. *p=0.01 when compared to the unstimulated Jurkat cells. *p=0.004 compared to non-stimulated Jurkat cells. No statistical differences were obtained when comparisons between cells treated with the H. pylori sonicate and cells treated with the mixture sonicate/NOHA were done (p=0.3).

FIG. 22: H. pylori arginase reduces the expression of the CD3ζ chain and proliferation of Jurkat cells. A. Arginase activity in WT ATCC 43504 H. pylori and the isogenic rocF(−) strain was measured as described in Materials and Methods. The mean±SE of three different experiments is shown. B. Expression of CD3ζ in Jurkat cells cultured with 50 μg/ml of sonicate from either the WT or the rocF(−) mutant H. pylori. The mean±SEM of six experiments is shown. *p=0.03 when compared to control cells; **p=0.0001 and p=0.008 when compared to controls and rocF(−), respectively. C. ³H-thymidine incorporation in Jurkat cells treated with the sonicates of either the WT or the rocF(−) mutant H. pylori. The figure shows the mean±SEM of three different experiments. *p=0.03 when compared to non-stimulated cells. No statistical difference was observed between non-stimulated cells and those treated with the rocF(−) sonicate (p=0.2).

FIG. 23: Live wild type H. pylori reduces the expression of CD3ζ in Jurkat cells. Jurkat cells were exposed to either wild type or arginase mutant H. pylori in a trans-well system (400 bacteria per Jurkat cell). The mean±SEM mean fluorescence intensity (MFI) of CD3ζ of the MFI of fluorescence in two experiments is shown. *p=0.02 as compared to non-stimulated cells. No statistical differences were observed between non-stimulated cells and those treated with the rocF(−) sonicate (p=0.7)

FIG. 24: H. pylori arginase blocks the normal recovery of CD3ζ expression in T-cells. After inducing the decreased expression of CD3ζ in T-cells by stimulation with anti-CD3/CD28, L-arginine (400 μM) and H. pylori sonicate (20 μg/ml) were added for additional 24 h. The CD370 expression was measured by flow cytometry. The mean±SEM of six different normal subjects is shown in the graph. The normal MFI for CD3ζ in freshly isolated T-cells was 40. *p=0.003 when compared to T-cells treated with H. pylori WT-derived sonicate.

FIGS. 25: A-D show the results of arginase production in tumors as described in Example VII.

FIG. 26: A shows the ncreased arginase production in the peripheral blood mononuclear cells of human patients with Renal Cell Carcinoma. B shows Arginase production in peripheral blood mononuclear cells of patients with pulmonary tuberculosis (PTB) compared with patients that are PPD+ or normal controls.

### DETAILED DESCRIPTION OF SPECIFIC EMBODIMENTS

The present invention is based on U.S. Provisional Patent Application No. 60/363,366, the entire content of which is hereby incorporated by reference.

The present invention provides methods and compositions for modulating immune responses in order to limit their deleterious effects on patients. According to this invention immune responses can be modulated by controlling the levels of arginine, and in particular by arginase I, systemically or in specific tissues according to the present invention. Arginine levels can be controlled according to the present invention by either providing arginine, or stimulating the production of arginase, preferably in a cell or tissue specific manner. Manipulating arginine levels allows immune responses to be enhanced or suppressed as needed. Additionally, the present invention provides several clinical and diagnostic methods which measure arginine or arginase levels, and thus the strength of an immune response.

The present invention also provides compositions and methods for treating a subject having suppressed T-cell function or proliferation. Generally, the suppression of T-cell function or proliferation will be the result of arginase activity, and in particular arginase I activity. Treatment of suppressed T-cell function or proliferation can include administering an effective amount of an arginase I inhibitor or an inhibitor of a cationic amino acid transporter Y+ receptor, such as a CAT-2B cationic amino acid transporter, to a patient in need thereof or administering an effective amount of an arginine provider, formulated as described herein, to a patient in need of such treatment. These two approaches can also be combined to further enhance the treatment efficacy. Generally, administering the arginase inhibitor, arginine provider, or both will generally increase the amount of arginine available to the T-cells. The increased arginine levels will, in turn, allow the T-cell to properly respond to antigenic stimuli thereby increasing T-cell function and/or proliferation. Without limiting the scope of the invention, unless otherwise stated, it is believed that by providing arginine to the T-cells, or limiting arginase activity, the T-cells are able to properly express the CD3ζ chain of the T-cell receptor after being bound by an antigen or anti-CD3 antibody. The present invention also provides a method of treating depressed circulating arginine levels by administering an arginase I inhibitor or an inhibitor of a cationic amino acid transporter Y+ receptor, such as a CAT-2B cationic amino acid transporter. The present invention also provides a method of treating diseases or disorders in which T-cell proliferation or function is impaired. The present invention also provides a method of treating diseases or disorders which have the characteristic or arginase production and/or depleted arginine levels.

The present invention thus also provides a method of treating the diseases, disorders or infections described herein comprising administering an effective amount of an arginase inhibitor, such as an arginase I inhibitor, an inhibitor of a cationic amino acid transporter Y+ receptor, such as a CAT-2B cationic amino acid transporter, to a patient in need thereof, regardless of the mechanism involved, as described herein. In the alternative, the method of treating these diseases, disorders or infections can also comprise administering an effective amount of an arginine provider, formulated as described herein, to a patient in need of such treatment. These two approaches can also be combined to further enhance the treatment efficacy.

Enhancing the immune function, such as T-cell function, of the subject suffering from one of these diseases, disorders or infections will help the subject to more effectively fight the condition and will also help to prevent the cell or pathogen causing the condition to escape the protective immune response of the subject.

In some embodiments, the present methods can be used to treat various cancers, including prostate, colon, breast or lung cancer. In other embodiments, the present invention can be used to treat to a cancers other than gastric or breast cancer.

In some embodiments of the present invention, the disease or disorder treated is not cachexia, heart disease, systemic hypertension, pulmonary hypertension, erectile dysfunction, autoimmune encephalomyelitis, chronic renal failure, a gastrointestinal motility disorder, a gastric cancer, breast cancer, reduced hepatic blood flow, or cerebral vasospasm. In other embodiments the present invention can be used to treat an infection, such as a parasitic infection. In some of these embodiments the infection is a parasitic infection other than leishmaniasis.

Although manipulation of arginase levels has been suggested to control the amount of arginine available for the production of NO via NOS pathways and thus control of muscle contraction, the present invention differs from these uses by manipulating arginase levels and exerting a direct effect on the immune system, either by increasing an immune response or decreasing an immune response. Additionally, the use of enzymes such as arginase have also been suggested to deplete arginine levels thereby starving cells, such as cancers or infectious organisms, that require arginine. In contrast, the present invention can inhibit arginase activity thereby increasing the amount of available arginine thereby allowing an effective enhancement of immune function in cells, such as macrophages, dendritic cells and T-cells, that would otherwise be immune suppressed due to low arginine availability. Additionally, in some embodiments the present methods do not simply treat the disclosed diseases or disorders by increasing nutritional levels of arginine, but instead have a direct effect on the immune system as discussed herein. Specifically, the present invention can specifically target the inhibitors of arginase I, the inhibitors of the cationic amino acid transporter Y+ receptor, such as CAT-2B, or arginine providers described herein to specific tissues. Alternatively, they can be administered systemically.

Previous publications have suggested that some conditions, diseases or disorders in which high arginase levels have been found can be treated with arginase inhibitors. However, these publication provided no mechanistic foundation for the effect that arginase levels had on overall immune response. Additionally, many of these publications simply report that arginase level is a marker for these diseases. Thus these publications cannot be extended to disorders in which there is general T-cell impairment or proliferation or all diseases or disorders which are known to have increased arginase levels.

Surprisingly, it has been found that some bacterial or viral infections can prevent an effective immune response against the bacteria or virus by producing or up-regulating arginase and/or depleting arginine levels and thus limiting the body's immune response against the infecting organism or virus. Thus, another aspect of the present invention involves increasing an immune response, such as a T-cell function or proliferation, in a subject suffering from a bacterial or viral infection, and in particular viral or bacterial infections which exhibit increased arginase levels. Treatment of these infections can include administering an effective amount of an arginase I inhibitor or an inhibitor of a cationic amino acid transporter Y+ receptor, such as a CAT-2B cationic amino acid transporter, to a patient in need thereof or administering an effective amount of an arginine provider, formulated as described herein, to a patient in need of such treatment. These two approaches can also be combined to further enhance the treatment efficacy. In some of the embodiments, the infection is not leishmaniasis.

Unexpectedly, it has been determined that arginine depletion is mostly a result of an increased arginase activity in tumor cells, infectious organisms, pathogens or in mononuclear cells (macrophages, monocytes or dendritic cells). Blocking of arginase completely prevents the depletion of arginine. However arginase is not released into the circulation in some cases and instead remains inside of the cells (tumors or macrophages). For aginine to enter the cell it is transported into the cell by cationic aminoacid transporters, mainly CAT-2B. Thus, arginase I activity and/or production, cationic aminoacid transporter activity or all of the forementioned can be targeted for inhibition. Blocking CAT-2B partially restores T-cell function by allowing z chain expression, but not to the level achieved by blocking arginase activity. This suggests that arginase is a more important component in the depletion of arginine.

Without limiting the scope of the invention, it is believed tumor or macrophages (in cancer) or microorganisms or macrophages (in infections) or macrophages alone (in autoimmunity), produce arginase and express CAT-2B receptors that make them uptake arginine and deplete it from the tissue microenvironment inducing molecular changes in the T cells including a decreased expression of the CD3z chain, an inability to translocate NFkBp65 and to upregulate Jak-3, all of which lead to an impaired immune response. This might also affect natural killer cells (NK) and possibly other macrophages. In addition, arginase may be produced by circulating macrophages or can be spilled into the circulation by tumor cells or macrophages undergoing apotosis leading to a depleted arginine levels in circulation and causing the same changes in T cells as mentioned above, that eventually lead to a decreased immune function. These deleterious effects can be treated or prevented using the present invention.

A. Enhancing the Immune Response Through Arginine Manipulation.

Increasing an immune response can be achieved according to the present invention by regulating the amount of arginine available to cells or tissue using at least two approaches. First, arginine availability in tissues or cells, such as T-cells or macrophages, can be enhanced so that arginine levels are increased. Increasing the level of arginine to cells can be achieved by delivering high concentrations of arginine providers such as arginine, arginine precursors, their pharmaceutical equivalents, arginine salts and combinations thereof to cells. This can be achieved in vivo by administering the arginine providers to a patient through any known method, including without limitation topically, intravenously or orally, depending upon the type of cells or tissue targeted. Preferably, the cells or tissue desired to have high levels of arginine available in their microenvironment are preferentially targeted by the administered arginine provider. Delivering arginine providers to specific tissues can be achieved by preferentially enhancing the delivery to specific cells, tissues or systems, such as the reticuloendothelial system, which includes the spleen, lymph nodes and the liver, and monocyte/macrophages, using suitable delivery vehicles described below.

Increasing the arginine levels in the microenvironment of the reticuloendothelial system or targeted cells and tissues can enhance the immune function of those cells, and in particular T-cells, thus boosting the overall immune response of a patient. This can be helpful in treating a wide variety of diseases and conditions, such as cancer, autoimmune diseases (systemic lupus erythematosus, rheumatoid arthritis), chronic infectious diseases (leprosy, tuberculosis, HIV/AIDS, chronic active hepatitis), trauma, burns, pulmonary diseases such as emphysema and chronic obstructive pulmonary disease (COPD) and the like. Very high local ranges of concentrations of arginine, which can be as much as two, five, ten, twenty, fifty, a hundred, a thousand, five thousand, ten thousand times or more over normal cell arginine concentrations (between 80 and 120 μM in normal individuals) can be achieved in cells targeted to have higher arginine levels. Preferably, the methods described herein will provide arginine levels to the T-cells or systemically that are about 40 μM or greater, for example 50 μM, 75 μM, 100 μM, 200 μM, 500 μM, 750 μM, 1000 μM, 2000 μM or greater. The only limit on the amount and corresponding increase of arginine which can be achieved is the result of delivery vehicle size and the physical characteristics, such as solubility, of the arginine providers. These high localized levels can be achieved without deleteriously affecting non-targeted cells. High localized concentrations of arginine can be used to overcome localized levels of arginase thereby restoring normal, if not providing enhanced, arginine levels and immune function in and around the targeted cells. These formulations are also suitable as adjuvants to increase the response to vaccines. In this manner fewer vaccinations are required and smaller amounts of antigenic material is required.

Because the utilization of L-arginine in the body can be controlled directly by arginine intake, which can occur through at least oral or intravenous supplementation, synthesis from other chemicals (such as pre-cursors, arginine equivalents, pharmaceutically acceptable salts of arginine, the amino acid glutamine, and combinations thereof), and through controlling the levels of active arginase all of these areas are preferably controlled according to the present invention.

Arginine levels in tissues can be increased to enhance an immune response, such as by:

1. Delivering high concentrations of arginine providers to the reticuloendothelial system and particularly the monocyte/macrophages using liposomal compositions described herein or other vesicles to enhance the immune response in patients with cancer, infectious diseases or other diseases where an increased immune response can help in the induction of a therapeutic or protective effect.

2. Using arginine-liposomes or other presentations of arginine to enhance the immune stimulation of vaccines (similar to other adjuvants).

Immune function of cells can also be enhanced by inhibiting the production or activity of arginase, and in particular arginase I. Inhibiting the production or effect of arginase will maintain the available levels of arginine for the immune response to proceed. Arginase inhibitors can be delivered to targeted cells, tissues and systems in the same manner as arginine providers as discussed herein. Preferably, the inhibition of arginase is achieved by specifically directing the arginase inhibitors directly at the cells of the reticuloendothelial system, and more specifically to the macrophages. Arginase inhibitors suitable for use in the present invention include, but are not limited to, amino acids (such as NG-hydroxy-L-arginine-[NOHA] which occurs naturally as a byproduct of nitric oxide production and is an excellent physiological inhibitor, N(omega)-hydroxy-nor-l-arginine [nor-NOHA]; ornithine, lysine, and norvaline), adrenergic blocking agents (such as beta-blockers including propanolol), cytokines (such as IL2, IFNg, IL12), other non-specific stimulators of macrophage function (for example, Bryostatin) and other compounds (i.e., borate). Preferably, arginase inhibitors used in the present methods preferentially inhibit arginase I production and/or activity as opposed to arginase II. Examples of such compounds include those disclosed in Colleluori et al., Biochemistry, 40:9356-9362 (2001). Other arginase inhibitors, and in particular arginase II inhibitors, are disclosed in U.S. Pat. No. 6,387,890. More preferably, the arginase inhibitors used herein specifically inhibit arginase I, and not arginase II, production and/or activity.

Surprisingly and unexpectedly, it has been discovered that the expression of the arginase I, which is inducible, is primarily responsible for the depletion of arginine, in the cells and surrounding environment, resulting in suppression of an immune response. In contrast, it is believed that arginase II, which is constitutively expressed, is responsible for most baseline arginase activity, but does not lead to overall arginine depletion and impairment of the immune response. Accordingly, preferentially inhibiting arginase I production and/or activity without inhibiting arginase II can be useful in overcoming arginine depletion which will result in less impairment of, and can possibly enhance, the immune response with minimal interference of other cellular processes requiring arginase II activity, such as in the production of NO.

The present methods and compositions are also useful for preventing or limiting infection by inhibiting arginase production and/or activity in infectious organisms. Unexpectedly, it has been elucidated that many infectious organisms use arginase to deplete the arginine levels of immune system cells thereby limiting the ability of the immune cells to respond to the infection. Accordingly, by inhibiting the arginase production and/or activity of these infectious organisms, the present methods can be used to treat or prevent such infections. In one embodiment, compositions containing arginase inhibitors can be directly targeted to the infections to limit the deleterious effect the arginase has on the immune response. Alternatively, immune system cells can be provided with enhanced levels of arginine and/or arginase inhibitors in order to overcome the arginase produced by the infectious organisms. The infections that can be treated include, but are not limited to tuberculosis, Helicobacter pylori, schistosomiasis and leprosy. Preferably, the methods described herein will provide arginine levels to the T-cells or systemically that are about 40 μM or greater, for example 50 μM, 75 μM, 100 μM, 200 μM, 500 μM, 750 μM, 1000 μM, 2000 μM or greater.

Cells which produce arginase, such as macrophages, tumor cells, transplanted cells, cells undergoing autoimmune reactions, liver cells, infectious organisms and the like, are particularly preferred targets for arginase inhibitors. Again, specific targeting of cells can be achieved as described elsewhere herein.

Enhancement of the immune response can also be achieved through inhibiting the production or activity of arginase. This can be achieved by at least:

1. Designing dietary supplements to enhance the immune response based on their ability to suppress arginase, or to increase the concentration of arginine. The latter can be achieved by increasing the absorption or delivery of arginine. Increased arginine concentration can be achieved by providing a higher concentration of arginine in dietary supplements or intravenous fluids, or by injecting arginine in an encapsulated form such as liposomes so as to have it delivered to the organs of the reticuloendothelial system including spleen, liver and lymph nodes, or to sites of inflammation.

2. Beta-blockers, such as propanolol, can be used to inhibit production of arginase and thus prevent the induction of anergy.

3. Arginase inhibitors can also be used to prevent the development of artherosclerotic plaques in blood vessels thus preventing or treating heart disease.

4. Arginase production can also be limited by inserting an antisense gene for an arginase gene in a tissue so that reduced amounts or no arginase enzyme is produced in the tissue. An antisense gene for arginase can be controlled by a constitutively expressed promoter, so arginase is constantly suppressed, or by an inducible promoter, so arginase production can be regulated as desired.

B. Compromising an Immune Response

Therapeutic benefit can also be achieved by decreasing the immune response of a patient. Anergy, or the complete lack of an immune response, can be achieved in a patient by increasing the amount and activity of arginase either systemically or more preferably in targeted cells and tissues. Induction of targeted anergy can have a therapeutic benefit in diseases where there is “chronic inflammation,” such as in autoimmune diseases such as lupus, rheumatoid arthritis, ulcerative colitis or Crohn's disease by interfering with localized immune response without impairing the systemic immune response of a patient. Induction of anergy is also useful in patients to prevent rejection, such as in patients receiving organ transplants and would also be helpful in preventing graft versus host disease or rejection of a transplanted organ. Beneficial results are preferably achieved by targeting arginase or the modulators of arginase production or activity specifically to the cells or tissues in which induction of anergy is desired. Thus a decreased immune response can be localized to specific tissues, such as the cartilage in rheumatoid arthritis or transplanted tissue in graft versus host disease, to achieve beneficial results without compromising the systemic immune response. The present invention provides therapeutic benefit through diminishing immune responses as well as completely preventing an immune response.

Increasing the production of arginase can be achieved with arginase stimulators. Preferred arginase stimulators for use in the present invention include, but are not limited to, beta-adrenergic agents (for example, epinephrine, norepinephrine, isoproterenol, dopamine, and salbutamol), cytokines (such as IL-4, IL-10, IL-13 and TGF-beta) and arginase substrates (i.e., arginine). Providing an arginase enzyme, which can be arginase I or arginase II from a natural or synthetic source, to the targeted cells is also useful for inducing anergy according to the present invention.

Therapeutic benefits which can be achieved by the induction of anergy or immunosuppression according to the present invention include:

1. A method of inducing T-cell anergy or immunosuppression by inducing the production of arginase by monocyte/macrophages, or decreasing the availability of L-arginine, to decrease or stop the induction of an immune response. The possible application of this technology can be to treat autoimmunity including lupus, arthritis, prevent or treat organ rejection, treat graft vs. host disease.

2. A method of making a transplant less immunogenic or resistant to rejection by treating the recipient with arginase or substances (medications, cytokines or others) that induce the production or increase the activity of arginase and therefore stop or prevent the development of an immune response against the transplanted organ. Preferably the arginase or substances that induce arginase production are targeted to the transplant through delivery vehicles and methods discussed herein.

3. A method of protecting an organ from rejection, or making a bone marrow used in transplantation resistant to rejection by inducing the production of arginase in the organ or bone marrow cells, or by transfecting such organ or cell transplants like pancreas, liver or bone marrow, with the arginase I gene. Techniques for inserting genes into the DNA of cells are known in the art and are useful for practicing the present invention. An arginase gene inserted into cells can be controlled by a constitutively expressed promoter, so arginase is produced constantly, or by an inducible promoter, so arginase production can be regulated as desired.

4. The use of catecholamines or catecholamine analogues (encapsulated or not encapsulated in liposomes) to increase the production of arginase and thus induce immune suppression or decrease the immune response. This can be used in diseases such as autoimmunity (lupus, rheumatoid arthritis, etc) or transplant rejection.

5. Additionally, blocking the synthesis of arginine from citrulline or other precursors with the resulting decrease in arginine can cause the development of immunosuppression or tolerance in cells or tissues.

6. Designing dietary supplements to decrease the immune response by enhancing the expression of arginase and/or decreasing the concentration of arginine.

Based upon the results set forth in the Examples, the present invention also provides a method for inducing the death of tumor cells, and in particular leukemia, by selectively depriving the tumor cells of arginine thus resulting in tumor cell death, presumably through apoptosis. This can be achieved by delivering high levels of arginase or drugs which deplete arginine specifically to the tumor cells thereby limiting the amount of arginine available to the tumor cells. This effectively results in starving the tumor cells of arginine and tumor cell death.

Additionally, the present invention also provides methods wherein arginine providers are delivered to certain tissues to enhance their immune response while at the same time the level of arginine in other tissues is decreased thereby inducing anergy in those targeted tissues.

The present invention also provides other uses, and in particular clinical tests and kits for performing these tests as set forth herein. Clinical tests for evaluating the state of immune competence in a patient, and measuring the efficacy of a treatment on a disease or condition are provided by the present invention.

1. The level of immunosuppression in an individual can be determined by measuring the level of arginase in specific fluids cells, such as the cytoplasm of peripheral blood cells or monocyte/macrophages or lymphocytes or in serum. As discussed in the examples below, the level of arginine has a direct correlation on T-cell function in the immune system. This aspect of the invention can be performed by, for example, isolating the fluid or cells of interest from the patient and measuring the arginase levels and/or activity through known arginase enzyme assays Arginase activity can be measured by the conversion of arginine to L-ornithine or urea. However, arginase can be directly determined by western blot or by ELISA. The expression of the arginase gene can be detected by PCR, by PCR ELISA and by Northern blot. These assays may include directly measuring the arginase level or measuring the ability of the fluid or cells to break down arginine. As a non-limiting example of this aspect of the invention, patients, and in particular trauma patients, which have high levels of arginase in their peripheral blood mononuclear cells and a low serum arginine levels are considered to be immunosuppressed.

2. The aggressiveness of a tumor can also be determined by measuring the ability of the tumor cells to produce arginase. To perform this aspect of the invention, preferably tumor cells are isolated and then the level of arginase and/or arginase activity is measured directly by detecting the amount of arginase enzyme produced or alternatively by assaying the ability of the cells to break down arginine. Alternatively, the presence of arginase, the level of arginase activity or the amount arginine in the serum of a patient with cancer can also be measured.

3. Tumors can also be measured for their sensitivity to immunotherapy by measuring the tumors ability to produce arginase in the presence different treatment regimes and pharmaceuticals to determine the most effective therapy against the tumor before subjecting the patient to the therapy. According to this feature of the invention tumor cells are isolated and subjected to proposed therapies including, but not limited to chemotherapy, radiation therapy, heat therapy, immunotherapy pharmaceuticals and the like, after which the cells ability to produce arginase is measured. This arginase production is compared against control cells from the tumor which have not been subjected to the therapy to determine the efficacy of the treatment on the tumor cells. Additionally, tumors in vivo can be made sensitive to immunotherapy or chemotherapy by blocking their ability to produce arginase using arginase inhibitors, for example nor-hydroxy arginine (NOHA) or N-nor-hydroxyarginine (nor-NOHA). This aspect of the invention involves targeting arginase inhibitors against the tumor cells as described elsewhere herein.

4. The efficacy of a treatment of a condition or disease, such as cancer, tuberculosis or trauma, can be measured by determining the ability of said treatment to decrease the production of arginase by the targeted cells or by cells of the immune system. This feature of the present invention is similar to the aspect of the invention discussed immediately above, however in this feature of the invention the cells are isolated prior to, and after, any treatment has been administered and the arginase production and/or activity of the cells is measure and compared. Accordingly, a determination can then be made whether the treatment has been effective. As a non-limiting example, patients with pulmonary tuberculosis that have no ζ chain expression have peripheral blood mononuclear cells with high arginase levels. The mononuclear cells are capable of releasing the arginase into culture medium, and presumably into serum. Treatments of tuberculosis which have been shown to be effective in providing an improved clinical response result in decreased arginase levels in the peripheral blood mononuclear cells. Conversely, in tuberculosis patients not responding to treatment, the arginase level in peripheral blood mononuclear cells remained high or increased.

5. The efficacy of immunosuppressive drugs or treatments can be measured by determining their ability to induce the production of arginase. According to this feature of the invention, immunosuppresive drugs are administered to either a patient or isolated cells of interest and the arginase level is measured and compared against the control wherein the arginase production and activity of cells without administered treatment is measured.

6. New immunosuppressive medications and treatments can be developed, and existing ones tested, by determining their ability to induce arginase production or activity in cells. This aspect of the invention can be performed by measuring the ability of known cell lines, for example Jurkat cells, to produce arginase before and after being subjected to medications and/or treatments.

7. New and existing immunostimulants can be tested by determining the ability of these medications to suppress the production and/or activity of arginase. This embodiment of the invention is similar to the ones previously mentioned although in this instance, the decrease in the production, activity or both of arginase will be measured instead of the increase in arginase or arginase activity.

8. Determining arginase level systemically or in specific cells may also indicate if a patient is likely to respond to treatment. For example, some tuberculosis patients having low starting arginase levels in peripheral blood mononuclear cells responded better to treatment than patients with higher levels of arginase. The level or of arginase may also be able to predict the stage or level or progression of disease in a patient. In general, higher levels of arginase correspond with later stages of disease.

Arginase content and activity in cells and fluid can be measured either directly, by measuring the amount of arginase, or indirectly, measuring the conversion of arginine to ornithine. Any suitable test may be used to measure the arginase level or activity in a fluid or cell. Preferred techniques for measuring arginase or arginase gene expression include ELISA, flow cytometry, Western blot analysis, Northern blot analysis and PCR.

C. Delivery Vehicles for Delivering Substances Which Modulate an Immune Response.

Delivery vehicles suitable for use in the present invention include without limitation liposomes and vesicles, such as multilamellar vesicles, small unilamellar vesicles, large unilamellar vesicles, sterically stabilized liposomes, immunoliposomes, virosomes, polymer coated liposomes, heterovesicular liposomes, and combinations thereof. These delivery vehicles are useful for delivering arginine, arginase inhibitors, arginase, stimulators of arginase production and DNA sequences containing an arginase gene or an anti-sense against the arginase gene to desired cells and tissues for targeted delivery. Production of such liposomes and vesicles are known in the art and can be suitably used in the present invention. Administration of these delivery vehicles can be given intravenously, intrathecally, subcutaneously, intraperitoneally, intrapleural, intra-articular, topically or by nebulization.

Passive targeting of the reticuloendothelial system with high relative concentrations of arginine providers to the reticuloendothelial system can be achieved with conventional liposomes because the circulating cells of the reticuloendothelial system readily remove and degrade unmodified liposomes. Thus, the contents of unmodified liposomes are readily available to cells of the reticuloendothelial system after the liposomes are removed and degraded. Longer circulating life for the liposomes can be achieved by treating the surface of the liposomes to resist uptake by cells or degradation, such as, for example, coating the liposomal surface with a polymer. Modification of the surfaces of liposomes with a non-ionic surface active agent, cationic surface active agent, anionic surface active agent, polysaccharides and derivatives thereof, polyoxyethylene derivatives etc. can be carried out as desired.

Active targeting of specific cells can also be achieved. Active targeting of a cell typically involves attaching a ligand to the surface of a liposome. Suitable ligands useful for liposome targeting include antibodies, enzymes, proteins, lectins, sugars, polysaccharides, peptides, aliphatic acids, and the like. Preferred ligands include antibodies which are specific for the certain cell targeted, such as macrophages, for an increase in arginine levels. For example, when the joints are targeted, as is desired for rheumatoid arthritis, antibodies against joint tissue, such as cartilage, can be attached to the surface of the liposome. In like manner, cancer cells can be targeted using the appropriate antibodies attached to the surface of liposomes. Production of polyclonal and monoclonal antibodies which are specific for certain cells and tissues are well known in the art. Ligands can be either attached covalently or non-covalently to the surface of the liposome although covalently bound ligands are preferred.

The solution used for suspending liposomes may be an acid, alkali, various buffers, physiological saline, amino acid infusions etc. in addition to water. Further, antioxidants, such as citric acid, ascorbic acid, cysteine, ethylenediaminetetraacetic acid (EDTA) etc., may also be added. Furthermore, preservatives such as paraben, chlorobutanol, benzyl alcohol, propylene glycol etc. may also be added. In addition, glycerin, glucose, sodium chloride etc. can also be added as agents for rendering the solution isotonic.

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

As the lipids for preparation of liposomes, mention is made of phospholipids, glyceroglycolipids, and sphingoglycolipids among which phospholipids are preferably used. Examples of such phospholipids include natural or synthetic phospholipids such as phosphatidylcholine, phosphatidylethanolamine, phosphatidylserine, phosphatidic acid, phosphatidylgycerol, phosphatidylinositol, lysophosphatidylcholine, sphingomyelin, egg yolk lecithin and soybean lecithin, as well as hydrogenated phospholipids etc.

The glyceroglycolipids include sulfoxyribosyldiglyceride, diglycosyldiglyceride, digalactosyldiglyceride, galactosyldiglyceride, glycosyldiglyceride, etc.

The sphingoglycolipids include galactosylcerebroside, lactosylserebroside, ganglioside etc. These are used singly or in combination. If necessary, sterols such as cholesterol as membrane stabilizer, tocopherol, etc. as antioxidant, stearylamine, dicetylphosphate, ganglioside, etc. as charged substances, may be used in addition to the lipid component

Controlled release formulations also are envisioned by the present invention. For example, there is a great deal of literature on liposomes that are useful to deliver proteins, the contents of the following U.S. patents are hereby incorporated by reference: U.S. Pat. Nos. 4,863,740, 4,877,561, 5,225,212, 5,007,057, 5,049,389, 5,023,087, 4,992,271, 4,962,091, 4,895,719, 4,855,090, 4,844,904, 4,781,871, 4,762,720, 4,752,425, 4,612,007, 5,292,524, 5,258,499, 5,229,109, 4,983,397, 4,895,719, and 4,684,521.

Additionally, the use of multivesicullar vesicles and microcapsules also are envisioned by the present invention, see WO 94/23697 and U.S. Pat. No. 5,102,872 respectively. The active agents described herein may be entrapped or conjugated to polymers and implanted in a patient to facilitate slow release. Examples of these technologies are shown in U.S. Pat. Nos. 5,110,596, 5,034,229, and 5,057,318, the respective contents of which are hereby incorporated by reference.

For production of the liposome preparation of the present invention, any method of preparing a known liposome preparation can be used. These methods of preparing a liposome preparation include, but are not limited to, the liposome preparation methods of Bangham et al. (J. Mol. Biol., 13, 238 (1965)), the ethanol injection method (J. Cell. Biol., 66, 621 (1975)), the French press method (FEBS Lett., 99, 210 (1979)), the freezing and thawing method (Arch. Biochem. Biophys., 212, 186 (1981)), the reverse phase evaporation method (Proc. Natl. Acad. Sci. (USA), 75, 4194 (1978)), and the pH gradient method (Biochim. Biophys. Acta, 816, 294 (1985); Japanese Patent Application Laid-Open Publication No. 165,560/95). Typically, production of liposomes containing the active agent, for example arginine providers, arginase, arginase inhibitors or arginase stimulators are produced by the following steps: dissolving a preferred active agent in a solvent suitable for dissolving the active agent to produce dissolved active agent; adding the dissolved active agent to a dissolved lipid suitable for formulation and delivery of drugs to produce a solution; and freezing and lyophilizing the solution. At this point, the solution may be stored frozen for later use or dissolved in sterile water to produce a suspension. For use, the lyophilized solution is suspended in appropriate volumes of sterile, distilled water. In addition, other methods of liposome preparation known in the art may be utilized, for example, rotary evaporation can be used instead of lyophilization.

The liposome preparation of the present invention obtained by e.g., the methods described above can be used as such, but can also be lyophilized after adding fillers such as mannitol, lactose, glycine etc. depending on the object of use, storage conditions etc. Lyoprotectants stabilizers such as glycerin etc. may also be added before lyophilization.

It is contemplated specifically that the pharmaceutical compositions of the present invention be used for the delivery of liposomes carrying arginine providers, arginase, arginase stimulators, arginase inhibitors, cationic amino acid transport inhibitors and the like to selected tissues or cells. A person having ordinary skill in this art would readily be able to determine, without undue experimentation, the appropriate dosages of these formulations. When used in vivo for therapy, the formulations of the present invention are administered to the patient in therapeutically effective amounts; i.e., amounts that eliminate or reduce the tumor burden. As with all pharmaceuticals, the dose and dosage regimen will depend upon the nature of the disease or disorder, the characteristics of the particular active agent (e.g., its therapeutic index), the patient, the patient's history and other factors. Again, dose and dosage regimen will vary depending on a number of factors known to those skilled in the art. See Remington's Pharmaceutical Science, 17th Ed. (1990) Mark Publishing Co., Easton, Pa.; and Goodman and Gilman's: The Pharmacological Basis of Therapeutics 8th Ed (1990) Pergamon Press.

D. Delivery of Nuceic Acid: Ex Vivo and In Vivo

Any means for the introduction of heterologous, such as arginase I, or antisense arginase, nucleic acids into host cells, especially eukaryotic cells, an in particular animal cells, preferably human or non-human mammalian cells, may be adapted to the practice of this invention. For the purpose of this discussion, the various nucleic acid constructs described herein may together be referred to as the transgene. Ex vivo approaches for delivery of DNA include calcium phosphate precipitation, electroporation, lipofection and infection via viral vectors. Two general in vivo gene therapy approaches include (a) the delivery of “naked”, lipid-complexed or liposome-formulated or otherwise formulated DNA and (b) the delivery of the heterologous nucleic acids via viral vectors. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g., with various lipid or liposome materials, may then be effected using known methods and materials and delivered to the recipient mammal.

While various viral vectors may be used in the practice of this invention, retroviral-, AAV- and adenovirus-based approaches are of particular interest. See, for example, Dubensky et al. (1984) Proc. Natl. Acad. Sci. USA 81, 7529-7533; Kaneda et al., (1989) Science 243,375-378; Hiebert et al. (1989) Proc. Natl. Acad. Sci. USA 86, 3594-3598; Hatzoglu et al. (1990) J. Biol. Chem. 265, 17285-17293 and Ferry, et al. (1991) Proc. Natl. Acad. Sci. USA 88, 8377-8381. The following additional guidance on the choice and use of viral vectors may be helpful to the practitioner.

i. Retroviral Vectors

Retroviruses are a class of RNA viruses in which the RNA genome is reversely transcribed to DNA in the infected cell. The retroviral genome can integrate into the host cell genome and requires three viral genes, gag, pol and env, as well as the viral long terminal repeats (LTRs). The LTRs also act as enhancers and promoters for the viral genes. The packaging sequence of the virus, (.PSI.), allows the viral RNA to be distinguished from other RNAs in the cell (Verma et al., Nature 389:239-242, 1997). For expression of a foreign gene, the viral proteins are replaced with the gene of interest in the viral vector, which is then transfected into a packaging line containing the viral packaging components. Packaged virus is secreted from the packaging line into the culture medium, which can then be used to infect cells in culture. Since retroviruses are unable to infect non-dividing cells, they have been used primarily for ex vivo gene therapy.

ii. AAV Vectors

Adeno-associated virus (AAV)-based vectors are of general interest as a delivery vehicle to various tissues, including muscle and lung. AAV vectors infect cells and stably integrate into the cellular genome with high frequency. AAV can infect and integrate into growth-arrested cells (such as the pulmonary epithelium), and is non-pathogenic.

The AAV-based expression vector to be used typically includes the 145 nucleotide AAV inverted terminal repeats (ITRs) flanking a restriction site that can be used for subcloning of the transgene, either directly using the restriction site available, or by excision of the transgene with restriction enzymes followed by blunting of the ends, ligation of appropriate DNA linkers, restriction digestion, and ligation into the site between the ITRs. The capacity of AAV vectors is about 4.4 kb. The following proteins have been expressed using various AAV-based vectors, and a variety of promoter/enhancers: neomycin phosphotransferase, chloramphenicol acetyl transferase, Fanconi's anemia gene, cystic fibrosis transmembrane conductance regulator, and granulocyte macrophage colony-stimulating factor (Kotin, R. M., Human Gene Therapy 5:793-801, 1994, Table I). A transgene incorporating the various DNA constructs of this invention can similarly be included in an AAV-based vector. As an alternative to inclusion of a constitutive promoter such as CMV to drive expression of the recombinant DNA encoding the fusion protein(s), an AAV promoter can be used (ITR itself or AAV p5 (Flotte, et al. J. Biol. Chem. 268:3781-3790, 1993)).

Such a vector can be packaged into AAV virions by reported methods. For example, a human cell line such as 293 can be co-transfected with the AAV-based expression vector and another plasmid containing open reading frames encoding AAV rep and cap under the control of endogenous AAV promoters or a heterologous promoter. In the absence of helper virus, the rep proteins Rep68 and Rep78 prevent accumulation of the replicative form, but upon superinfection with adenovirus or herpes virus, these proteins permit replication from the ITRs (present only in the construct containing the transgene) and expression of the viral capsid proteins. This system results in packaging of the transgene DNA into AAV virions (Carter, B. J., Current Opinion in Biotechnology 3:533-539, 1992; Kotin, R. M, Human Gene Therapy 5:793-801, 1994)). Methods to improve the titer of AAV can also be used to express the transgene in an AAV virion. Such strategies include, but are not limited to: stable expression of the ITR-flanked transgene in a cell line followed by transfection with a second plasmid to direct viral packaging; use of a cell line that expresses AAV proteins inducibly, such as temperature-sensitive inducible expression or pharmacologically inducible expression. Additionally, one may increase the efficiency of AAV transduction by treating the cells with an agent that facilitates the conversion of the single stranded form to the double stranded form, as described in Wilson et al., WO96/39530.

Concentration and purification of the virus can be achieved by reported methods such as banding in cesium chloride gradients, as was used for the initial report of AAV vector expression in vivo (Flotte, et al. J. Biol. Chem. 268:3781-3790, 1993) or chromatographic purification, as described in O'Riordan et al., WO97/08298.

For additional detailed guidance on AAV technology which may be useful in the practice of the subject invention, including methods and materials for the incorporation of a transgene, the propagation and purification of the recombinant AAV vector containing the transgene, and its use in transfecting cells and mammals, see e.g., Carter et al, U.S. Pat. No. 4,797,368 (Jan. 10, 1989); Muzyczka et al, U.S. Pat. No. 5,139,941 (Aug. 18, 1992); Lebkowski et al, U.S. Pat. No. 5,173,414 (Dec. 22, 1992); Srivastava, U.S. Pat. No. 5,252,479 (Oct. 12, 1993); Lebkowski et al, U.S. Pat. No. 5,354,678 (Oct. 11, 1994); Shenk et al, U.S. Pat. No. 5,436,146 (Jul. 25, 1995); Chatterjee et al, U.S. Pat. No. 5,454,935 (Dec. 12, 1995), Carter et al WO 93/24641 (published Dec. 9, 1993), and Flotte et al., U.S. Pat. No. 5,658,776 (Aug. 19, 1997).

iii. Adenovirus Vectors

Various adenovirus vectors have been shown to be of use in the transfer of genes to mammals, including humans. Replication-deficient adenovirus vectors have been used to express marker proteins and CFTR in the pulmonary epithelium. The first generation E1a deleted adenovirus vectors have been improved upon with a second generation that includes a temperature-sensitive E2a viral protein, designed to express less viral protein and thereby make the virally infected cell less of a target for the immune system (Goldman et al., Human Gene Therapy 6:839-851, 1995). More recently, a viral vector deleted of all viral open reading frames has been reported (Fisher et al., Virology 217:11-22, 1996). Moreover, it has been shown that expression of viral IL-10 inhibits the immune response to adenoviral antigen (Qin et al., Human Gene Therapy 8:1365-1374, 1997).

DNA sequences of a number of adenovirus types are available from Genbank. The adenovirus DNA sequences may be obtained from any of the 41 human adenovirus types currently identified. Various adenovirus strains are available from the American Type Culture Collection, Rockville, Md., or by request from a number of commercial and academic sources. A transgene as described herein may be incorporated into any adenoviral vector and delivery protocol, by the same methods (restriction digest, linker ligation or filling in of ends, and ligation) used to insert the CFTR or other genes into the vectors. Hybrid Adenovirus-AAV vectors represented by an adenovirus capsid containing selected portions of the adenovirus sequence, 5′ and 3′ AAV ITR sequences flanking the transgene and other conventional vector regulatory elements may also be used. See e.g., Wilson et al, International Patent Application Publication No. WO 96/13598. For additional detailed guidance on adenovirus and hybrid adenovirus-AAV technology which may be useful in the practice of the subject invention, including methods and materials for the incorporation of a transgene, the propagation and purification of recombinant virus containing the transgene, and its use in transfecting cells and mammals, see also Wilson et al, WO 94/28938, WO 96/13597 and WO 96/26285, and references cited therein.

Generally the DNA or viral particles are transferred to a biologically compatible solution or pharmaceutically acceptable delivery vehicle, such as sterile saline, or other aqueous or non-aqueous isotonic sterile injection solutions or suspensions, numerous examples of which are well known in the art, including Ringer's, phosphate buffered saline, or other similar vehicles. Preferably, in gene therapy applications, the DNA or recombinant virus is administered in sufficient amounts to transfect cells at a level providing therapeutic benefit without undue adverse effects. Optimal dosages of DNA or virus depends on a variety of factors, as discussed elsewhere, and may thus vary somewhat from patient to patient. Again, therapeutically effective doses of viruses are considered to be in the range of about 20 to about 50 ml of saline solution containing concentrations of from about 10⁷ to about 10¹⁰ pfu of virus/ml, e.g., from 10⁸ to 10⁹ pfu of virus/ml.

iv. Host Cells

This invention is particularly useful for the engineering of animal cells and in applications involving the use of such engineered animal cells. While various mammalian cells may be used, including, by way of example, equine, bovine, ovine, canine, feline, murine, and non-human primate cells, human cells are of particular interest. Among the various species, various types of cells may be used, such as hematopoietic, neural, glial, mesenchymal, cutaneous, mucosal, stromal, muscle (including smooth muscle cells), spleen, reticulo-endothelial, epithelial, endothelial, hepatic, kidney, gastrointestinal, pulmonary, fibroblast, and other cell types. Of particular interest are cells of the reticulo-endothelial system. Also of interest are stem and progenitor cells, such as hematopoietic, neural, stromal, muscle, hepatic, pulmonary, gastrointestinal and mesenchymal stem cells

The cells may be autologous cells, syngeneic cells, allogeneic cells and even in some cases, xenogeneic cells with respect to an intended host organism. The cells may be modified by changing the major histocompatibility complex (“MHC”) profile, by inactivating beta2-microglobulin to prevent the formation of functional Class I MHC molecules, inactivation of Class II molecules, providing for expression of one or more MHC molecules, enhancing or inactivating cytotoxic capabilities by enhancing or inhibiting the expression of genes associated with the cytotoxic activity, or the like.

In some instances specific clones or oligoclonal cells may be of interest, where the cells have a particular specificity, such as T-cells and B-cells having a specific antigen specificity or homing target site specificity.

v. Introduction of Constructs into Animals

Cells which have been modified ex vivo with the DNA constructs may be grown in culture under selective conditions and cells which are selected as having the desired construct(s) may then be expanded and further analyzed, using, for example, the polymerase chain reaction for determining the presence of the construct in the host cells and/or assays for the production of the desired gene product(s). Once modified host cells have been identified, they may then be used as planned, e.g., grown in culture or introduced into a host organism.

Depending upon the nature of the cells, the cells may be introduced into a host organism, e.g., a mammal, in a wide variety of ways. Hematopoietic cells may be administered by injection into the vascular system, there being usually at least about 10⁴ cells and generally not more than about 10¹⁰ cells. The number of cells which are employed will depend upon a number of circumstances, the purpose for the introduction, the lifetime of the cells, the protocol to be used, for example, the number of administrations, the ability of the cells to multiply, the stability of the therapeutic agent, the physiologic need for the therapeutic agent, and the like. Generally, for myoblasts or fibroblasts for example, the number of cells will be at least about 10⁴ and not more than about 10⁹ and may be applied as a dispersion, generally being injected at or near the site of interest. The cells will usually be in a physiologically-acceptable medium.

Cells engineered in accordance with this invention may also be encapsulated, e.g., using conventional biocompatible materials and methods, prior to implantation into the host organism or patient for the production of a therapeutic protein. See e.g., Hguyen et al, Tissue Implant Systems and Methods for Sustaining viable High Cell Densities within a Host, U.S. Pat. No. 5,314,471 (Baxter International, Inc.); Uludag and Sefton, 1993, J Biomed. Mater. Res. 27(10):1213-24 (HepG2 cells/hydroxyethyl methacrylate-methyl methacrylate membranes); Chang et al, 1993, Hum Gene Ther 4(4):433-40 (mouse Ltk- cells expressing hGH/immunoprotective perm-selective alginate microcapsules; Reddy et al, 1993, J Infect Dis 168(4):1082-3 (alginate); Tai and Sun, 1993, FASEB J 7(11):1061-9 (mouse fibroblasts expressing hGH/alginate-poly-L-lysine-alginate membrane); Ao et al, 1995, Transplanataion Proc. 27(6):3349, 3350 (alginate); Rajotte et al, 1995, Transplantation Proc. 27(6):3389 (alginate); Lakey et al, 1995, Transplantation Proc. 27(6):3266 (alginate); Korbutt et al, 1995, Transplantation Proc. 27(6):3212 (alginate); Dorian et al, U.S. Pat. No. 5,429,821 (alginate); Emerich et al, 1993, Exp Neurol 122(1):37-47 (polymer-encapsulated PC12 cells); Sagen et al, 1993, J Neurosci 13(6):2415-23 (bovine chromaffin cells encapsulated in semipermeable polymer membrane and implanted into rat spinal subarachnoid space); Aebischer et al, 1994, Exp Neurol 126(2):151-8 (polymer-encapsulated rat PC12 cells implanted into monkeys; see also Aebischer, WO 92/19595); Savelkoul et al, 1994, J Immunol Methods 170(2):185-96 (encapsulated hybridomas producing antibodies; encapsulated transfected cell lines expressing various cytokines); Wirm et al, 1994, PNAS USA 91(6):2324-8 (engineered BHK cells expressing human nerve growth factor encapsulated in an immunoisolation polymeric device and transplanted into rats); Emerich et al, 1994, Prog Neuropsychopharmacol Biol Psychiatry 18(5):935-46 (polymer-encapsulated PC12 cells implanted into rats); Kordower et al, 1994, PNAS USA 91(23):10898-902 (polymer-encapsulated engineered BHK cells expressing hNGF implanted into monkeys) and Butler et al WO 95/04521 (encapsulated device). The cells may then be introduced in encapsulated form into an animal host, preferably a mammal and more preferably a human subject in need thereof. Preferably the encapsulating material is semipermeable, permitting release into the host of secreted proteins produced by the encapsulated cells. In many embodiments the semipermeable encapsulation renders the encapsulated cells immunologically isolated from the host organism in which the encapsulated cells are introduced. In those embodiments the cells to be encapsulated may express one or more chimeric proteins containing component domains derived from proteins of the host species and/or from viral proteins or proteins from species other than the host species. For example in such cases the chimeras may contain elements derived from GAL4 and VP16. The cells may be derived from one or more individuals other than the recipient and may be derived from a species other than that of the recipient organism or patient.

Instead of ex vivo modification of the cells, in many situations one may wish to modify cells in vivo. For this purpose, various techniques have been developed for modification of target tissue and cells in vivo. A number of viral vectors have been developed, such as adenovirus, adeno-associated virus, and retroviruses, as discussed above, which allow for transfection and, in some cases, integration of the virus into the host. See, for example, Dubensky et al. (1984) Proc. Natl. Acad. Sci. USA 81, 7529-7533; Kaneda et al., (1989) Science 243,375-378; Hiebert et al. (1989) Proc. Natl. Acad. Sci. USA 86, 3594-3598; Hatzoglu et al. (1990) J. Biol. Chem. 265, 17285-17293 and Ferry, et al. (1991) Proc. Natl. Acad. Sci. USA 88, 8377-8381. The vector may be administered by injection, e.g., intravascularly or intramuscularly, inhalation, or other parenteral mode. Non-viral delivery methods such as administration of the DNA via complexes with liposomes or by injection, catheter or biolistics may also be used.

In accordance with in vivo genetic modification, the manner of the modification will depend on the nature of the tissue, the efficiency of cellular modification required, the number of opportunities to modify the particular cells, the accessibility of the tissue to the DNA composition to be introduced, and the like. By employing an attenuated or modified retrovirus carrying a target transcriptional initiation region, if desired, one can activate the virus using one of the subject transcription factor constructs, so that the virus may be produced and transfect adjacent cells.

The DNA introduction need not result in integration in every case. In some situations, transient maintenance of the DNA introduced may be sufficient. In this way, one could have a short term effect, where cells could be introduced into the host and then turned on after a predetermined time, for example, after the cells have been able to home to a particular site.

### EXAMPLES

### Example I

Regulation of Arginine Levels in Jurkat Cells.

The data presented in this Example shows that the absence of L-arginine induces a profound decrease in the overall expression of the CD3ζ. The decreased CD3ζ chain expression results in a decreased response to antigenic stimuli and thus a decreased immune response. These changes are not produced by an increased apoptosis and do not affect the expression of the IL-2 receptor.

Jurkat cells were cultured for 3 days in C-RPMI or arginine free-RPMI with or without stimulation (αCD3 plus PHA) and the proliferation was assessed by ³H-thymidine incorporation. As shown in Table 1, Jurkat cells cultured in the absence of L-arginine had a significantly lower proliferation with or without stimulation, as compared to those cultured in C-RPMI (containing about 1000 μM L-arginine).

TABLE 1
(³H)Thymidine incorporation (CPM) in Jurkat cells cultured
for 3 days in RPMI with or without L-arginine (P < 0.00001)

| Medium | Unstimulated | Stimulated^{a} |
|---|---|---|
| C-RPML | 70801 | 125452 |
| Arg.-free RPMI | 57774 | 49884 |

^{a}Stimulation with 30 ng/ml αCD3 plus 1 μg/ml PHA

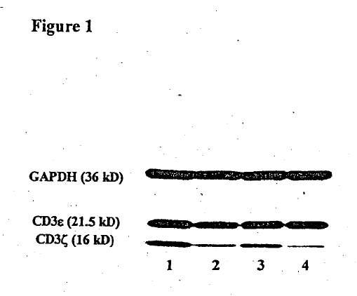

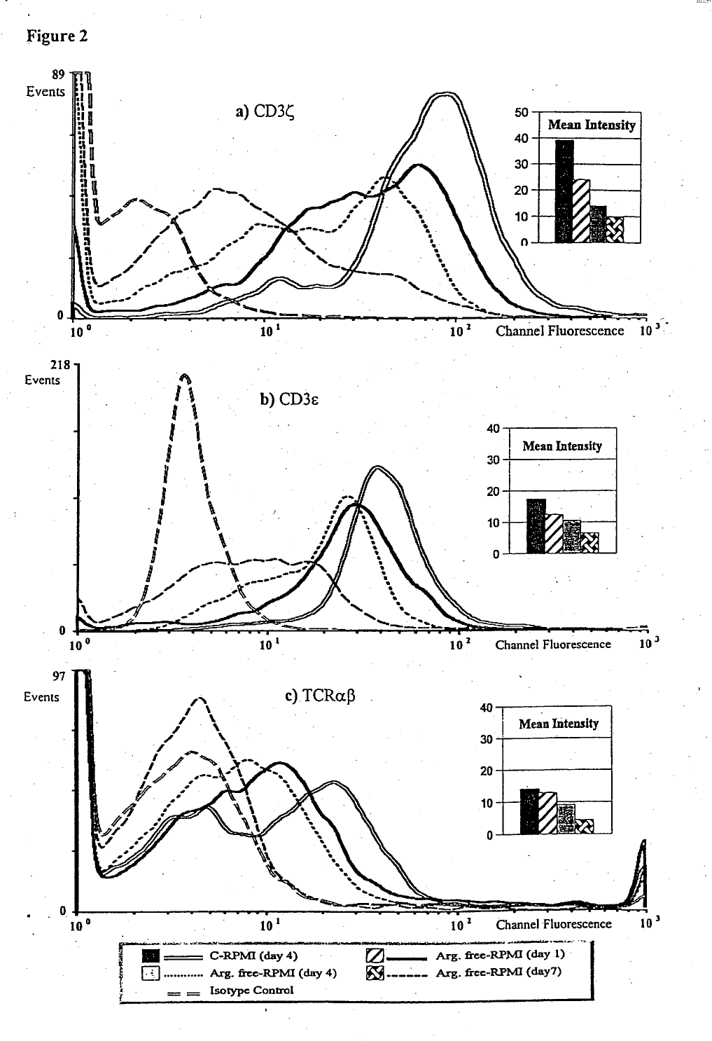

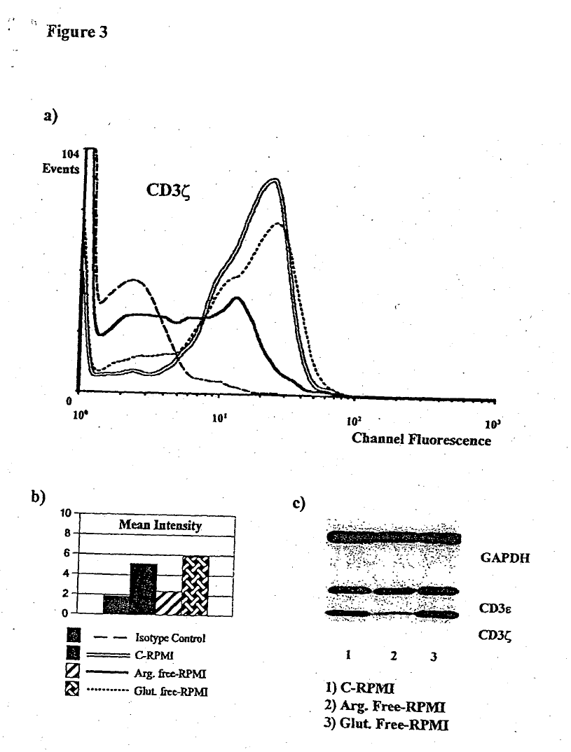

Next, it was tested whether L-arginine depletion altered the expression of the key elements of the T-cell antigen receptor and IL-2 receptor in Jurkat cells. As seen in FIG. 1 , the expression of CD3ζ chain was markedly decreased in Jurkat cells cultured for 2 and 4 days in arginine free-RPMI (lanes 2 and 4) when compared with those cultured in C-RPMI (lanes 1 and 3). The decreased expression of ζ chain was not due to an overall protein degradation since the expression of GAPDH and CD3ε was virtually unchanged over the same period of time. The decreased expression of CD3ζ was confirmed by flow cytometry (FIG. 2a), demonstrating a gradual and progressive decrease in the intensity of CD3ζ expression in Jurkat cells cultured in arginine free-RPMI. The initial decrease in CD3ζ could be seen as early as 24 hours, but continued up to 7 days in culture as compared to its expression in Jurkat cells cultured in C-RPMI that remains unchanged (data not shown). Flow cytometry also showed a decreased membrane expression of CD3ε and TCRαβ (FIGS. 2b&c) similar to that of CD3ζ. However, the cytoplasmic levels of CD3ε protein, as seen by Western blot, remained constant in cells cultured in arginine free-RPMI (FIG. 1). This suggested that the decrease in the membrane expression of the CD3ε and TCRαβ in the absence of L-arginine could be explained by a decrease in CD3ζ chain, preventing the assembly and expression of the TCR, and was not because of a decrease in the cytoplasmic levels of CD3ε protein. The decrease in CD3ζ was specifically induced by the depletion of L-arginine, since culture of Jurkat cells in glutamine free-RPMI did not result in any changes in its expression or that of the other TCR proteins (FIG. 3).

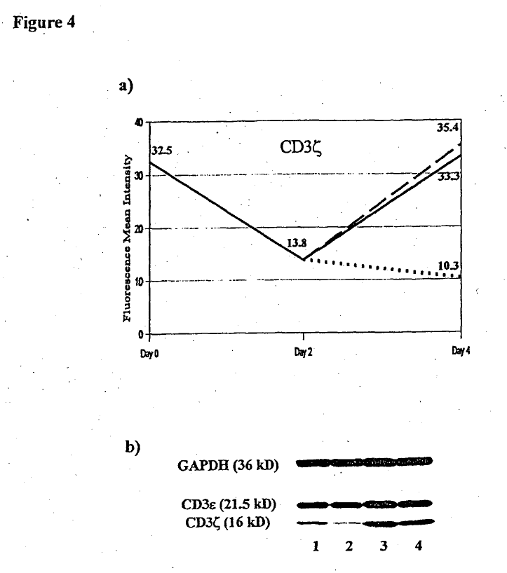

The loss of CD3ζ was fully reversible when L-arginine was replenished in the tissue culture medium. Reappearance of CD3ζ chain occurred either by adding L-arginine alone at a concentration of 100 μM (FIGS. 4a, dashed line, and 4b, lane 3), or by replacing the TCM with C-RPMI (FIGS. 4a, solid line, and 4b, lane 4). This effect was specific for L-arginine since the transfer of Jurkat cells that had lost CD3ζ, into fresh TCM without L-arginine (supplementing other amino-acids except L-arginine), failed to induce a re-expression of ζ chain and the TCR (data not shown). The data discussed is representative of at least five experiments in each case.

### Example II

Effect of L-arginine Depletion on the Re-expression of CD3ζ Chain and the T-cell Receptor After Antigen Stimulation of Normal T Cells.

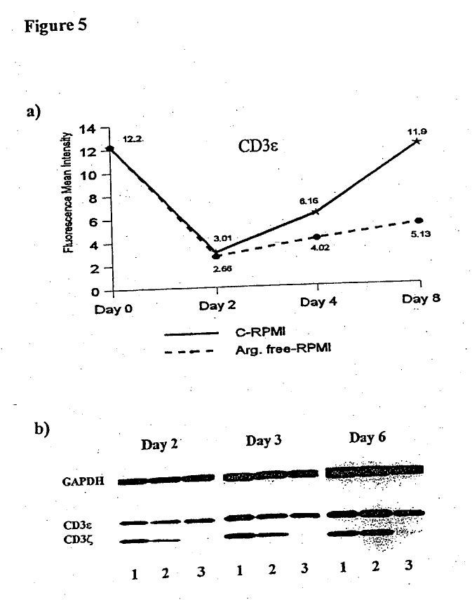

Upon binding to antigen, the T-cell receptor is internalized, followed by its re-expression within the following 48 hours. The effect of L-arginine depletion on the re-expression of CD3ζ chain and the T-cell receptor after antigen stimulation was tested. Normal T lymphocytes stimulated with anti-CD3 in C-RPMI showed the normal cycle of down regulation of the T-cell receptor followed by its reexpression 48 hours later and a complete recovery after 8 days (FIG. 5a, solid line). However, if the cells were cultured in arginine free RPMI the TCR failed to be re-expressed suggesting that CD3ζ chain had not been re-expressed. These changes in the membrane expression of the TCR were paralleled by an initial decrease in the cytoplasmic levels of the CD3ζ protein (FIG. 5b, day 2, lane 2) which is re-expressed after 48-72 hours (FIG. 5b, days 3 & 6, lane 2). During this process there are minimal changes in the level of cytoplasmic CD3ε protein. In contrast, T-cells cultured in arginine free-RPMI after the antigen stimulation, were unable to re-express the CD3ζ chain (FIG. 5b, day 6, lane 3). This prevented the TCR from being re-expressed on the cell membrane (FIG. 5a, dashed line), although the concentration of CD3ε protein in the cytoplasm remained unchanged as seen by Western blot (FIG. 5b).

The depletion of arginine, however, did not affect the normal expression and mitogen induced upregulation of the IL-2 receptor chains. Jurkat cells cultured in arginine free-RPMI and stimulated with anti-CD3 plus PHA showed an increase in the IL-2 receptor α, β, and γ_{c} chains similar to the cells cultured in C-RPMI (Table 2). Likewise, the production of IL-2, although slightly lower in arginine free-RPMI, did not appear to be significantly decreased (Table 3). Testing for the expression of the tyrosine kinases associated with the IL-2 receptor demonstrated a temporary decrease in the expression of Jak-3, (but not Jak-1) during the first 48 hours in culture, that was spontaneously normalized after this period of time (data not shown).

TABLE 2
Increased expression of IL-2 receptor chains α, β, and γ as
measured by the percentage of positive cells upon stimulation^{a} of
Jurkat cells cultured in RPMI 1640 with or without L-arginine

| | | C-RFMI | | Arg.-free RPMI | | |
|---|---|---|---|---|---|---|
| IL-2R | Days in | Un- | Stimu- | Un- | Stimu- | |
| Chain | Culture | stimulated | lated^{a} | stimulated | lated^{a} | P |
| α | 1 | 2.76 | 26.1 | 2.30 | 23.9 | 0.73 |
| | 2 | 2.76 | 11.7 | 1.94 | 17.3 | 0.44 |
| β | 1 | 5.70 | 15.1 | 3.00 | 15.3 | 0.38 |
| | 2 | 4.14 | 21.4 | 3.50 | 26.3 | 0.78 |
| γ_{c} | 1 | 4.80 | 8.4 | 2.78 | 6.8 | 0.87 |
| | 2 | 3.58 | 9.82 | 2.28 | 12.3 | 0.36 |

^{a}Stimulation with 30 ng/ml αCD3 plus 1 μg/ml PHA
^{b}P comparing stimulated cells with and without L-arginine

TABLE 3
IL-2 production (pg/ml in supernatant) by Jurkat cells upon
stimulation for 24 h after being cultured in RPMI 1640 with
or without L-arginine (P = 0.10)

| Days in | C-RPMI | | Arg.-free RPMI | |
|---|---|---|---|---|
| Culture | Unstimulated^{a} | Stimulated | Unstimulated^{a} | Stimulated |
| 1 | ND^{b} | 350 | ND | 300 |
| 2 | ND | 240 | ND | 150 |

^{a}Stimulation with 30 ng/ml αCD3 plus 1 μg/ml PHA
^{b}ND, not detectable

Without limiting the scope of this invention it is believed that modulating levels of the amino acid L-arginine can regulate the expression of specific signal transduction proteins in tissues, and in particular T-cells, and thus control their immunological competence.

In summary, based on the data from this example, modulation of arginine levels in cultures of Jurkat cells or normal T lymphocytes shows that:

1. An absence or very low levels of L-arginine decrease the expression of the T-cell receptor ζ chain (CD3ζ), resulting in a down-regulation of the complete T-cell receptor complex from the cell membrane (5 proteins plus the CD3ζ dimer). This is shown in FIGS. 1 and 2 of the manuscript by Taheri et al.

2. The absence of L-arginine also decreases the ability of Jurkat cells to proliferate to an antigenic stimuli. (Table 1)

3. This phenomenon is specific for L-arginine since the culture of Jurkat cells in glutamine-free tissue culture medium does not change the expression of the T-cell receptor or of the CD3ζ chain. (FIG. 3)

4. The replenishment of L-arginine into the tissue culture medium induces a re-expression of the CD3ζ chain and of the T-cell receptor. (FIG. 4).

5. This process does not interfere with other important receptors such as the IL2 receptor, which increases normally after stimulation, even in the absence of L-arginine. Thus the phenomenon is not that of a generalized process of protein degradation in the cell. (Table 2).

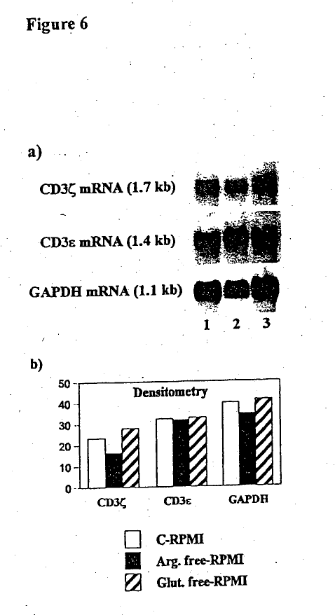

6. The absence of L-arginine produces a decreased expression of the CD3ζ-RNA (FIG. 6). Further research has shown that this is due to a diminished stability of the RNA, and possible post-transcriptional regulation (data available).

7. Although there are no immediate signs of apoptosis (programmed cell death) during the first 24-48 hours, long-term culture (>4 days) in the absence of L-arginine does increase the number of apoptotic Jurkat cells. This would suggest that certain leukemic cell lines might be exquisitely sensitive to the absence of certain amino acids or micronutrients that do not affect normal T-cells. This provides a therapeutic approach to the treatment of certain malignancies including cancers, including without limitation leukemia. (Data not shown).

Accordingly, these results provide a specific subset of cells and/or tissues which can be targeted in order to treat diseases which result in the loss of T-cell receptor ζ chain, non-limiting examples of which include cancer, autoimmune diseases, chronic infections, trauma and burns. However, the scope of the present invention is not limited to diseases or conditions wherein the T-cell receptor ζ chain, or another signal transduction protein, is lost as a result of disease progression.

### Example III

Regulation of Arginine Levels in T-cells.

The T-cells in Example II were treated under the same conditions as the Jurkat cells of Example I. The data in normal T-cells shows the following:

1. The culture of normal T-cells in the absence of L-arginine does not in itself produce any changes in the CD3ζ or the T-cell antigen receptor.

2. Once stimulated with an antigen, the T-cell receptor is internalized and normally re-expressed on the membrane within 24-48 hours. If this is done in the absence of L-arginine the T-cell receptor and the CD3ζ chain are not re-expressed. This renders the T-cell non-functional since it does not have a TCR with which to recognize antigen.

3. The stimulation of T-cells in the absence of arginine also prevents the up-regulation of certain cytokine genes such as IFN-g, IL4 and IL5, but not IL2, nor the IL2 receptor. Therefore, the absence of arginine appears to control the production of some cytokines.

Similar results have been obtained with the addition of arginase into the tissue culture medium. Jurkat cells lose the expression of z chain with concentrations of arginase as low as 1 unit/ml.

Surprisingly and unexpectedly, the loss of signal transduction molecules in T-cells requires a combination of both the absence of L-arginine and antigen stimulation, and production of certain cytokines, such as IFN-g, IL4 and IL10, was decreased by the absence of arginine. Additionally, the induction of signal transduction defects by the inclusion of arginase in the tissue culture medium was surprising. Therefore, these results confirm that limiting the availability of arginine to otherwise functional T-cells results in the induction of anergy which can thus be taken advantage of for therapeutic benefit as described in the present invention.

The conditions, data and results of Examples I -III are further discussed in Clin Cancer Res. Mar;7(3 Suppl):958s-965s (2001).

### Example IV

This Example Demonstrates That L-arginine Modulates CD3ζ Expression and T-cell Function in Activated T-cells

### Materials and Methods

T-cell Preparations:

Peripheral blood mononuclear cells (PBMC) from normal donors were separated over Ficoll-Paque (Pharmacia Biotech, Uppsala, Sweden). PBMC were enriched over T-cell enrichment columns (R&D Systems, Minneapolis, Minn.). After 10 min. of incubation, the columns were washed and the obtained enriched T-cells were counted and tested for surface markers by flow cytometry. The resultant enriched T-cell preparation contained >95% CD3⁺ cells, <3% CD16⁺ cells and <1% each of B (CD19⁺) cells and monocytes (CD14⁺).

Tissue Culture Media and Cell Cultures:

Tissue culture dishes (100 mm) (Corning, N.Y.) were coated overnight at 4° C. with PBS containing 10 μg/ml of anti-CD3 (OKT-3 Ortho Pharmaceutical, Raritan, N.J.) and washed once with cold phosphate buffer saline (PBS). Two million purified T-cells were resuspended in complete RPMI-1640 (RPMI) containing approximately 1140 μM of L-arginine (BioWhittaker, Walkersville, Md.) or in RPMI-1640 without L-arginine (Arg-free-RPMI) (GIBCO, Invitrogen, Grand Island, N.Y.) containing 100 ng/ml of anti-CD28 (Becton Dickinson, San Jose, Calif.) and added to plates. Control media included RPMI without L-glutamine or L-glycine. Unstimulated T-cells were placed in the same culture conditions as controls. All cultures were supplemented with 10% Fetal Calf Serum (FCS) (Hyclone, Road Logan, Utah), 20 mM of Hepes buffer (GIBCO, Life Technologies, Inc. Gaithersburg, Md.) and 4 mM of L-glutamine (BioWhittaker).

Flow Cytometry:

To determine CD3ζ expression, purified T-cells were incubated for 15 min at 4° C. with anti-CD3-FITC or the isotype control (Beckman-Coulter, Miami, Fla.) at 1 μg of antibody/10⁶ cells. Cells were washed and re-suspended PBS containing digitonin at 500 μg/ml (Wako, BioProducts, Richmond, Va.) and 2.5 μg of anti-CD3ζ-PE antibody (Beckman-Coulter, Miami, Fla.). The cells were incubated for 8 minutes at 4° C., washed with PBS, re-suspended in PBS for analysis. For surface markers 3×10⁵ T-cells were plated on a 96 well/U-bottom plates (Corning, Corning, N.Y.) and incubated with 1 μg of isotype control, CD3, CD14, CD19, CD16, CD69 or CD25 antibodies (Becton-Dickinson, San Jose, Calif.) for 15 minutes at 4° C., followed by two washes with PBS containing 2% bovine serum albumin (BSA) (Sigma, St. Louis, Mo.) then fixed in 1% paraformaldehyde. For apoptosis, T-cells were stained in the surface with 2 μg of Apo 2.7 antibody (Beckman-Coulter, Miami, Fla.) or stained after cell surface permeabilization with 100 μg/ml digitonin. Fluorescence analysis was done using a Coulter-EPICS flow cytometer (Beckman-Coulter, Miami, Fla.).

Western Blot:

Briefly, 10×10⁶ cells were lysed in Triton X-100 buffer with protease inhibitors as described before. Zea, A. H., M. T. Ochoa, P. Ghosh, D. L. Longo, W. G. Alvord, L. Valderrama, R. Falabella, L. K. Harvey, N. Saravia, L. H. Moreno, and A. C. Ochoa. 1998. Changes in expression of signal transduction proteins in T lymphocytes of patients with leprosy. Infect.Immun. 66:499-504. Lysates were electrophoresed in 14% Tris-glycine gels (Novex, San Diego, Calif.) and transfer to PDFV membranes (Novex), blocked and immunobloted with the different antibodies and detected by horseradish peroxidase conjugate antibodies and ECL (Amersham, Arlington Height, Ill.) and autoradiographed on X-OMAT AR films (Eastman, Kodak, Rochester, N.Y.).

Cytokine Production:

Supernatants from the T-cells cultures were collected at 48, 72 and 96 h and tested for IL2, IFNγ, IL4, IL5, and IL-10 production by ELISA. Briefly, 96 well plates (Immulon IV, Dynatech, Burlington, Mass.) were coated with the respective monoclonal antibody (Biosource, Camarillo, Calif.) and the culture supernatants incubated for 30 min. The reaction was detected by biotin-streptavidin conjugated with horseradish peroxidase (Pharmingen, San Diego, Calif.) using 3,3′,5,5′-tetramethylbenzidine (Roche, Indianapolis, Ind.) as a substrate. The reaction was stopped with 0.8 M sulfuric acid and the absorbances were read at 450 nm. The minimum level of cytokines detectable by the assay was 30 pg/ml.

Proliferation Assay:

1×10⁵ unstimulated or stimulated cells/well in media RPMI with or without arginine were plated. After 24, 48, and 72 hours of culture, 0.5 μCi of ³H-thymidine/well (NEN) was added and allowed to incubate for 18 hours at 37° C. Each condition was tested in triplicate. The cells were lysed by freeze and thawing harvested onto a Unifilter-96 GF/B (Packard, Meriden, Conn.) and counted using a TOPCOUNT Microplate Scintillation Counter (Packard, Meridien, Conn.).

Isolation of RNA for Northern Blots and Ribonuclease Protection Assay:

Total RNA was extracted from 10⁷ T-cells by lysis with TRIzol (GIBCO). Ten micrograms of total RNA was electrophoresed under denaturing conditions, blotted onto nytran membranes (Schleicher & Schuell Inc, Keene, N.H.), and cross-linked by UV irradiation. Membranes were prehybridized at 42° C. in ULTRAhyb buffer (Ambion, Austin, Tex.) and hybridized overnight with 1×10⁶ cpm/ml of [³²P]-labeled specific probes. Membranes were then washed three times, and radiographed at −70° C. using Kodak BIOMAX-MR films (Eastman Kodak). The murine cDNA glyceraldehide-3 phosphate dehydrogenase (GAPDH) (Clontech, Palo Alto, Calif.) and the human CD3ζ (a kind gift from Dr. Allan Weissman, NIH, Bethesda, Md.) were labeled by random priming using a RediPrime Kit (Amersham) and α-[³²P]-dCTP 3,000 Ci/mmol (NEN). To test mRNA synthesis inhibition, stimulated T-cells were cultured in presence or absence of L-arginine for 12, 24 and 48 hours. At that time points 5 μg/ml of actinomycin D (Sigma) were added to the cultures and samples for RNA extraction were taken at 0, 2, 4, and 8 hours. All signal intensities were normalized to GAPDH. Densitometry analysis was performed to analyze the band intensities.

For the ribonuclease protection assay (RPA), 5 μg of the RNA were mixed with the templates (Pharmingen, San Diego, Calif.) and incubated first at 90° C. allowing the temperature to ramp down slowly to a 56° C. The samples were treated with RNAse followed by proteinase K treatment. After extraction with phenol-chlorophorm, the sample was precipitated in 100% ethanol for 30 min at −70° C. The sample was recovered by centrifugation a 12000 rpm and resuspended in loading buffer. The samples were separated on a polyacrylamide gel containing 8M Urea. The gel was dried and exposed to a BIO-MAX films (Eastman, Kodak).

Radiolabeling and Pulse Experiments:

Ten million T-cells were cultured for 24, 48 and 72 h in RPMI or a Arg-free-RPMI media were harvested and resuspended in 2 ml of Methionine-free RPMI or Methionine-free-Arg-free-RPMI (GIBCO). After a 1 h of starvation the cells were labeled for 6 h with 1 mCi of [³⁵S]-methionine (NEN) in RPMI or Arg-free-RPMI media with 5% dialyzed FCS. Cells were washed twice in cold PBS and lysed in 1% digitonin and 0.12% Triton X-100 buffer plus protease inhibitors. Cell lysates were incubated with protein G-Sepharose beads (Pharmacia, Upsala, Sweden) coated with 20 μg of OKT-3 (Ortho-Pharmaceutical) The immunoprecipitates were subjected to one-dimensional non-reducing SDS-polyacrylamide gel electrophoresis (PAGE). Gels were dried and exposed to Kodak BIOMAX MR (Eastman Kodak).

Lysosome and Proteasome Inhibition:

To inhibit lysosome function 2×10⁶ T-cells cultured in the presence or absence of L-arginine, were treated with 1 μM bafylomycin A1 (Calbiochem, San Diego, Calif.) or 1 μM folimycin (Calbiochem) during the course of the experiments begining at time 0 (start of the cultures), 24, 48 and 72 hours after stimulation. To inhibit proteasome activity 2.5 μM lactacystin (Calbiochem) was added to the cultures at the same time points. DMSO was used as a control vehicle in all the cases. The CD3ζ expression was tested by flow cytometry 24 hours after the addition of the inhibitors.

Statistical Analysis:

The significance of changes were calculated by student's t test using the Graph-Pad statistical program (Graph-Pad, San Diego, Calif.)

### Results of This Example

Depletion of L-arginine Blocks the Normal TCR Cycling and CD3ζ Re-expresion in Activated T-cells:

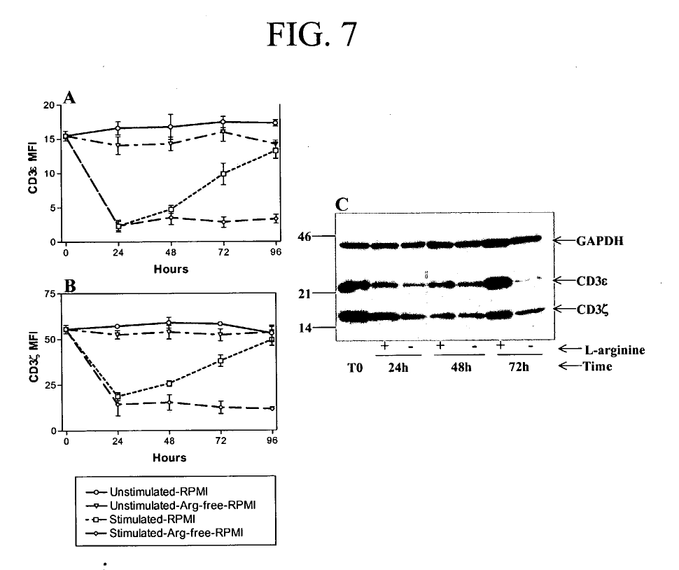

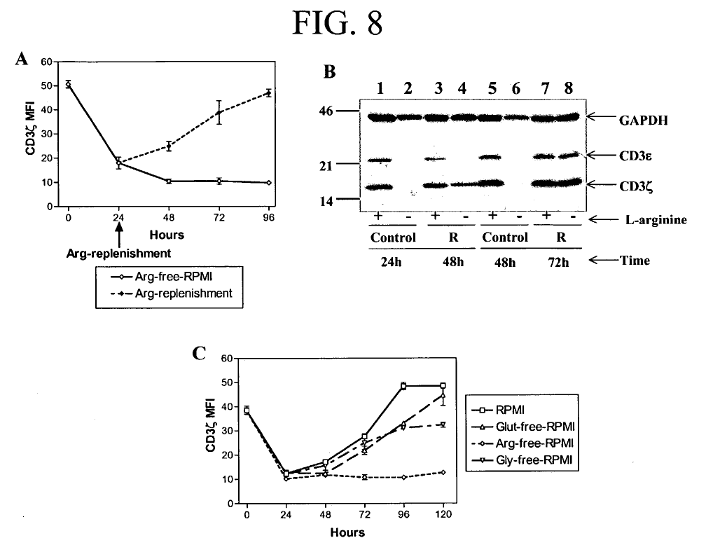

T-cells stimulated with cross-linked anti-CD3 plus anti-CD28 and cultured in conventional RPMI 1640, which contains 1140 μM L-arginine (RPMI), showed a normal cycle of down-regulation and re-expression of the CD3ε (FIG. 7A) and CD3ζ (FIG. 7B) over 48 to 72 hours. T-cells stimulated and cultured in Arg-free-RPMI showed a similar down-regulation of CD3ζ at 24 hours, however, they failed to recover the expression of CD3ζ and consequently of the TCR over the following 72 hours (FIG. 7B, 7A). Unstimulated T-cells did not show any changes in the expression of the CD3ε or CD3ζ when cultured in Arg-free-RPMI or RPMI. Western blot analysis confirmed the results seen by immunofluorescence (FIG. 7C). The addition of L-arginine (1140 μM) to the culture media resulted in the rapid re-expression of CD3ζ (FIG. 8A) even when L-arginine was added as late as 72 hours after stimulation (data not shown). The effect of L-arginine on CD3ζ was amino acid specific since the depletion of other acids such as L-glutamine, L-glycine (FIG. 8B) or L-leucine and L-lysine (data not shown), did not alter the cycle of internalization and re-expression of CD3ζ after antigen stimulation. In addition, this effect was selective for the TCR since other receptors such as the IL2 receptor increased normally after stimulation in T-cells cultured in L-arginine free medium (Table below).

L-arginine Starvation Markedly Reduces Cell Proliferation and Cytokine Production:

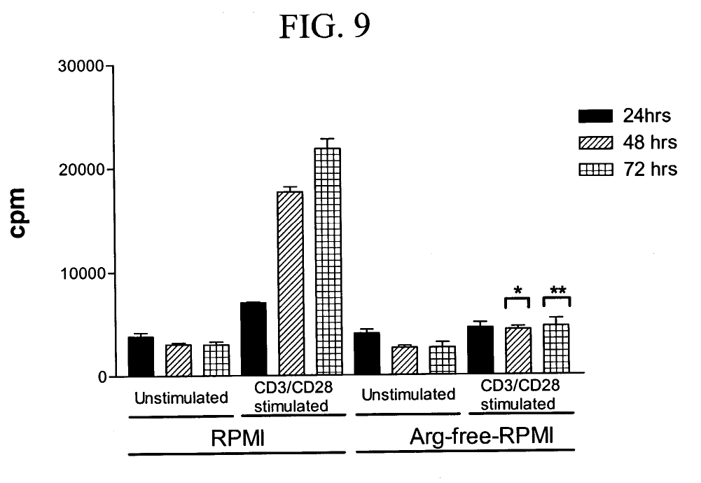

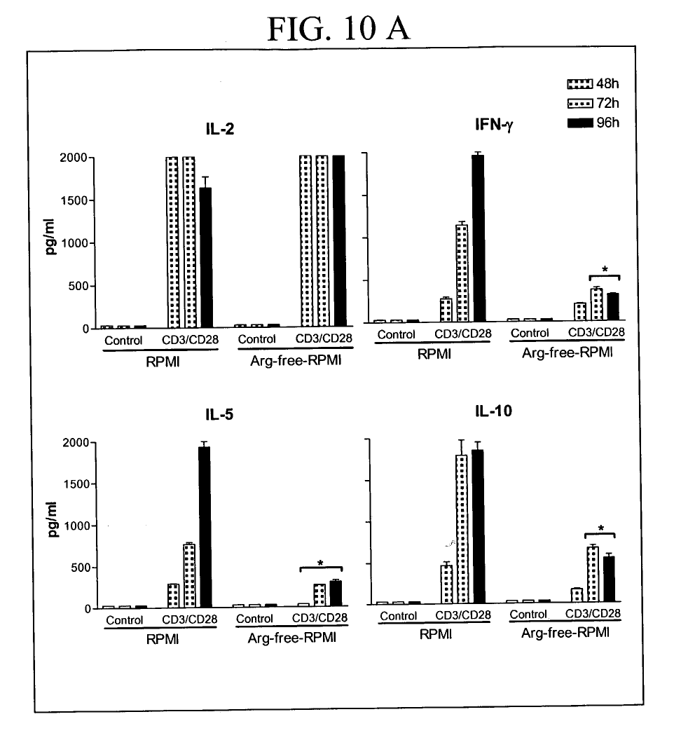

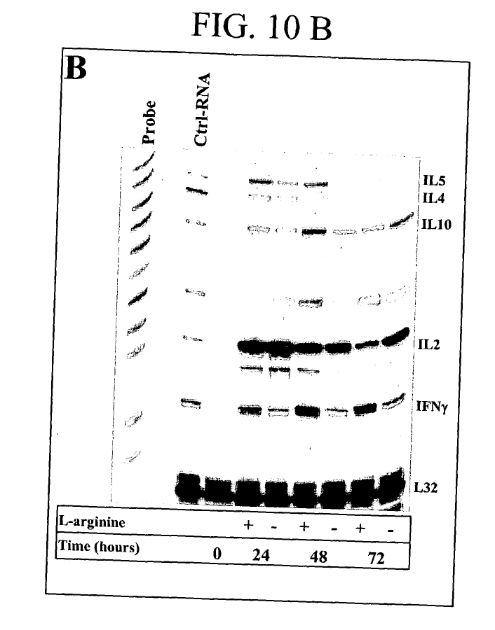

The absence of L-arginine affected other T-cell functions was also tested. As seen in FIG. 9, T-cells stimulated and cultured in Arg-free-RPMI up to 72 h, had a negligible [³H]-thymidine incorporation as compared to T-cells cultured in RPMI. In addition T lymphocytes cultured in the absence of L-arginine had a significantly (p<0.001) decreased production of IFNγ, IL4, IL5 and IL10 after 48 h in culture. Interestingly however, the production of IL2 was not decreased by the absence of L-arginine (FIG. 10A). A ribonuclease protection assay (RPA) confirmed these findings and demonstrated that the expression of RNA for IFNγ, IL4, IL5 and IL10 were decreased in T-cells stimulated and cultured in Arg-free-RPMI, while IL2 mRNA levels were not different in cells cultured in the presence or absence of L-arginine (FIG. 10B).

Mechanisms Leading to a Decreased Expression of CD3ζ in Arginine Starvation:

Without limiting the scope of this invention, it is postulated that the inability to re-express CD3ζ in T-cells cultured in the absence of L-arginine could be caused by several mechanisms including an increased T-cell apoptosis, an increased degradation of the CD3ζ protein, a decrease in CD3ζ mRNA expression or stability, or a diminished protein synthesis.

There was no increase in the expression of apoptosis markers Apo 2.7 (Table I in this example) or Anexin V (data not shown) in T-cells cultured in the absence of L-arginine, when compared to T-cells cultured in RPMI. In addition, the viability of the T-cells (by trypan blue exclusion) cultured in the absence of L-arginine was always >92% at all time points in the experiments.

Western blots and flow cytometry analysis did not show differences in the phosphorylation of CD3ζ (Table 5), indicating that the phosphorylated form of CD3ζ does not increase in the absence of the non-phosphorylated form.

TABLE 5
Expression of CD3ζ and phospho-CD3ζ in stimulated T
cells cultured in presence or absence of L-arginine

| | 24 | 48 | 72 | |
|---|---|---|---|---|
| Control | CD3ζ | 42.4 | 53.4 | 53.8 |
| | CD3-p-ζ | 10.8 | 10.9 | 2.71 |
| A+ | CD3ζ | 5.71 | 12.4 | 24.1 |
| | CD3-p-ζ | 5.18 | 2.32 | 2.62 |
| A− | CD3ζ | 3.59 | 4.78 | 5.99 |
| | CD3-p-ζ | 2.83 | 5.18 | 4.11 |

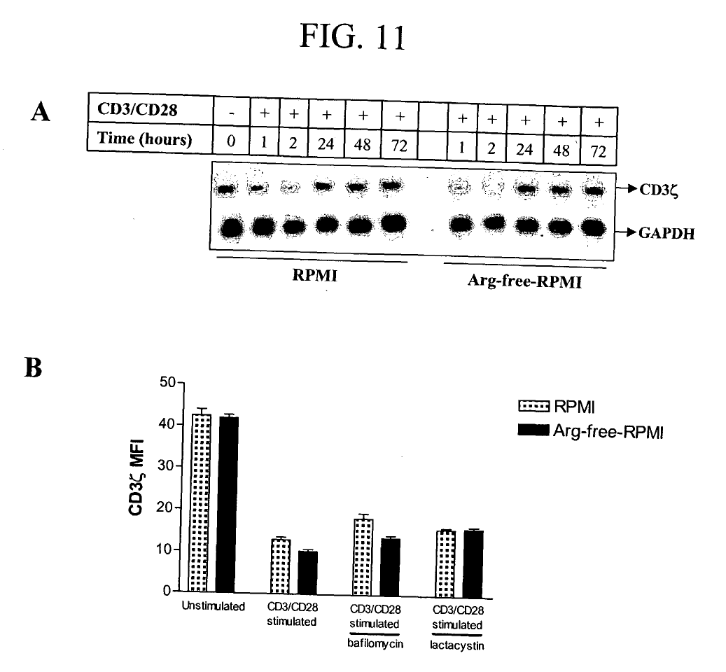

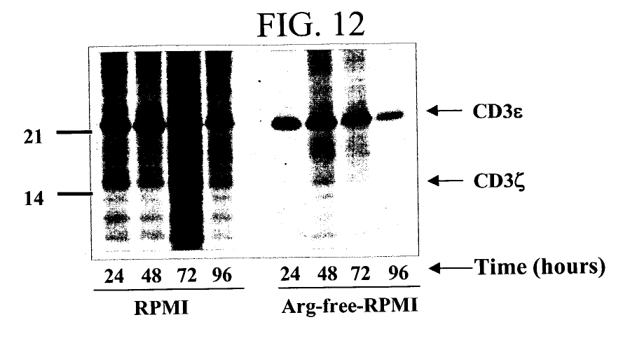

The inhibition of proteosome or lysosome function by the use of lactacystin or bafilomicyn failed to prevent the decrease of CD3ζ upon stimulation. The addition of these drugs after 24 hours caused a minor but not significant increase in CD3ζ which remained below the levels seen in unstimulated T-cells (FIG. 11A).

Northern blots also failed to demonstrate differences in CD3ζ mRNA expression between T-cells cultured in Arg-free-RPMI or RPMI. There were no differences in the mRNA expression at 24, 48 and 72 hours that could explain the decreased expression of the CD3ζ protein at 24 h in the cells cultured in RPMI and the decreased expression of CD3ζ at 24, 48 and 72 h in cells cultured in Arg-free-RPMI (FIG. 11B). However, a slight decrease in mRNA expression was observed in cells cultured in Arg-free-RPMI. To determine whether the stimulation of T-cells in absence of L-arginine influenced the stability of the CD3ζ mRNA, stimulated T-cells were cultured for 12, 24 and 48 hours, then actinomycin D was added and the measurement of the expression of CD3ζ mRNA at different time points, were assessed. As seen in FIG. 11C, there were no differences in CD3ζ mRNA stability at different time points between the cells culture in Arg-free media and those cultured in RPMI.

All together, this data therefore suggests that the decreased expression of in CD3ζ in stimulated T-cells cultured in Arg-free-RPMI was not caused by cell apoptosis, protein degradation, transcriptional or posttranscriptional mechanisms.

The possibility that the absence of L-arginine blocked the synthesis of CD3ζ was also tested. Pulse experiments were done with T-cells stimulated and cultured in RPMI or Arg-free-RPMI media for 24, 48 and 72 hours and labeled with [³⁵S] methionine. After 48 h T-cells cultured in RPMI had re-expressed CD3ε and CD3ζ, while cells cultured in Arg-free-RPMI still had a decreased expression of CD3ζ. The cell lysates were immunoprecipitated with anti-CD3 moAb or an irrelevant antibody and analyzed in a one dimensional SDS-PAGE. As shown in FIG. 12, T-cells stimulated and cultured in RPMI undergo a minor decrease in CD3ζ at 24 hours but overall maintained a normal rate of synthesis for CD3ε and CD3ζ. In contrast, T-cells stimulated and cultured in absence of L-arginine, show an inability to synthesize new CD3ζ, but not CD3ε. Thus the results could suggest a possible regulation of CD3ζ at the translational level.

### Discussion of Results

T-cells are activated following the recognition by the TCR of antigenic peptides bound to MHC molecules. The TCR is a multimeric protein complex consisting of the clonotypic αβ heterodimer, the CD3 γδε chains and the ζ homodimer. The αβ heterodimer is responsible for for specific recognition of the antigens, whereas the associated CD3 and ζ homodimer are responsible for signaling by the complex. The regulation of CD3ζ expression is mostly regulated by antigen stimulation. Activation of T-cells results in the modulation of TCR CD3ζ by internalization, degradation and recycled back to cell surface.

Decreased expression of CD3ζ chain have been reported in T-cells from patients with cancer, infectious diseases and autoimmune diseases. Different mechanisms for this decrease include apoptosis, production of peroxide by macrophages and chronic stimulation among others. In this example, the absence of the amino acid L-arginine produce in stimulated T-cells a sustained down-regulation of the CD3ζ chain possibly by a reduction in the synthesis rate of the protein.

L-arginine is a semi-essential amino acid that plays an important role in the immune response. In macrophages, L-arginine is the substrate for nitric oxide (NO) synthesis by the inducible nitric oxide synthase (iNOS) and the production of ornithine and urea by arginase. Increased arginase in serum can deplete plasma levels of L-arginine in vivo following liver transplantation and trauma, resulting in a decreased T-cell proliferation that can be reversed by the infusion of L-arginine. Previous studies have shown that certain amino acids such as L-cysteine, L-glutamine, L-lysine and L-methionine are essential for cell growth and proliferation after antigen stimulation. Furthermore, it has been demonstrated that the depletion of most essential amino acids (glutamine, histidine, lysine, proline) leads to apoptosis. Jurkat cells cultured in the absence of L-arginine presented a decreased levels of CD3ζ expression, low T-cell proliferation but normal production of IL2. This could be explained by the fact that other amino acids might play an important role in the regulation of cytokine production. For instance, L-glutamine enhances the production of cytokines by T-cells, including IL2, IL10 and IFNγ. Furthermore, a recent study demonstrated that adequate concentrations of glutamine increase a Th1 response. However, in this study the presence of L-glutamine in the tissue culture media did not normalize the production of cytokines that were decreased by the absence of L-arginine. Interestingly when resting T-cells were cultured in Arg-free-RPMI the expression of CD3ζ and T-cell functions were normal. However, when stimulated T-cells were cultured in RPMI media the T-cells presented the normal cycle of internalization (24 h) and re-expression on the surface (48-72 h). In contrast, an absence of L-arginine CD3ζ chain remained downregulated during the time periods established in the experiments. The decreased expression of CD3ζ was accompanied by low T-cell proliferation, low production of IFNγ, IL4, IL10 cytokines, but did not alter the production of IL2. Thus, the deprivation of L-arginine in stimulated T-cells selectively alters the expression of certain proteins essential to T-cell activation, alterations that are not seen when the T-cells are cultured in absence of other amino acids such as leucyne, glycine or glutamine. However, absence of L-arginine do not completely shut down all T-cell functions since the replenishment or addition of L-arginine to the tissue culture media (even at concentrations as low as 100 μM, produces the recovery of CD3ζ and T-cell functions 24 hours after its addition. In vitro models have demonstrated that the addition of L-arginine to tissue culture medium increases the response of CD8⁺ T-cells to antigen stimulation and increases the relative density of the TCR on the cell membrane (11). In addition, one possible explanation for the recovery of CD3ζ is that early T-cell activation pathways such as Ca⁺⁺ flux and tyrosine phosphorylation are not impaired by the depletion of L-arginine starvation (data not shown). Furthermore, in addition to the determination of CD3ζ, the expression of other signal transduction proteins involved in T-cell activation in absence of L-arginine need to be tested.

In this example some of the possible mechanisms postulated previously to explain the decreased expression of CD3ζ chain have been studied. One of the mechanisms postulated to explain the decreased expression of CD3ζ is apoptosis. Furthermore, it has been demonstrated that the depletion of certain essential amino acids leads to apoptosis. However, in this example L-arginine depletion does not appear to affect T-cell viability nor does it induce apoptosis. Antigen stimulation in the absence of L-arginine causes major alterations in the cycle of internalization and re-expression of the TCR by inducing a prolonged and sustained decrease in the expression of CD3ζ. Under normal conditions, stimulation of the TCR by antigen, anti-CD3 antibody or PKC activators causes its internalization. The seven protein chains that make up the TCR then either enter a recycling pathway or are sorted to and degraded in lysosomes or proteasomes. For CD3ζ, internalization and lysosomal degradation is followed by the synthesis of new CD3ζ protein, assembly of new TCRs in the endoplasmic reticulum and the expression of new receptors on the cell membrane. The initial internalization results in a reduction of the TCR levels that appear to play an important role in extinguishing the activation signal and reducing T-cell responsiveness to new antigen stimulation. In this example L-arginine depletion did not increase proteosomal or lysosomal degradation of the CD3ζ chain (FIG. 11A), since the use of lysosomal and proteosomal inhibitors did not increase the expression of CD3ζ.

Recent publications by the present inventors indicate that L-arginine starvation induced a rapid decrease in CD3ζ expression in the Jurkat T-cell line. Although the mechanisms to explain this decrease en CD3ζ expression are not yet clear, they appear to be associated with a decrease in RNA expression parallel to a short half life of CD3ζ mRNA. However the work presented here suggests that the mechanisms by which L-arginine may regulate CD3ζ expression in normal T lymphocytes is significantly different from the Jurkat T-cell line. First, the absence of L-arginine does not induce alterations in TCR expression in resting T-cells. This process appears to be protein specific since other proteins such as the early activation markers CD69 (data not shown), the IL2 receptor and the production of IL2 (FIG. 10A) are not altered by the absence of L-arginine and secondly, L-arginine starvation does not affect the expression of CD3ζ mRNA nor mRNA stability in normal T-cells, but does decrease the RNA expression for certain cytokines including IL4, IL5, IL10 and IFNγ. It is unclear whether similar mechanisms might be responsible for the changes in cytokine mRNA in normal T-cells. Preliminary data also show that L-arginine may modulate the translocation of the p65 subunit of the NF-κB nuclear transcription factor (data not shown), which is important in cytokine production.

Taken all together, these results indicate that the decreased expression of CD3ζ is not due to transcriptional or posttranscriptional mechanisms. Instead, it appears that the decreased expression of CD3ζ chain in absence of L-arginine is due to a lack in its synthesis. Metabolic labeling show a clear decrease in the novo CD3ζ protein synthesis in T-cells cultured in Arg-free-RPMI when compared to those cultured in RPMI. The molecular mechanisms involved in the control of gene expression by amino acid starvation have been extensively studied in yeast. The effect of amino acid starvation in mammalian cells is still unclear. General amino acid starvation of mammalian cells results in a pronounced fall in the overall rate of protein synthesis, associated with an increased phosphorylation of the alpha-subunit of the initiation factor eIF-2, which in turn impairs the activity of the guanine nucleotide exchange factor, eIF-2B. It is known that human primary T-cells are metabolic quiescent with little ongoing DNA, RNA or protein synthesis. The low level of protein synthesis rate in quiescent T-cells is associated with low levels of initiation factors in these cells. The rate of protein synthesis increase 2-4 fold after mitogenic stimulation, and has been reported that the mRNA and protein levels for several translation initiation factors increase during T-cell activation. It is possible speculate that the T-cells cultured in absence of L-arginine no proliferate, thus are quiescent and therefore, the levels of initiation factors is low. These translational mechanisms need to be further investigated. However, how these mechanisms may impair CD3ζ synthesis is currently unknown. It is most unclear which of the multiple steps in protein translation may be altered by the depletion of L-arginine.

It is also possible that L-arginine starvation may impair protein folding. A review of the Gene Bank Database shows that all of the arginine's of CD3ζ are located in the intra-cytoplasmic portion of the chain, while in other surface proteins that are not affected by L-arginine starvation such as the IL2R chains, the arginines are distributed throughout the protein. It is possible but yet unclear whether the distribution of arginine residues in ζ chain affects the synthesis of this protein in absence of arginine.

The findings described here could also be important in explaining the diminished expression of certain T-cell signal transduction proteins described in patients with cancer, chronic infections and some autoimmune diseases. A decreased expression of CD3ζ and other signal transduction proteins has been described in patients with cancer, leprosy, AIDS and autoimmune diseases such as lupus and arthritis. The similarity in the alterations in T-cell signal transduction proteins in spite of diversity in the pathophysiology of these diseases suggests a common mechanism as a cause for these changes. Several mechanisms have been shown to cause a decrease in CD3ζ chain expression including the induction of T-cell apoptosis by Fas-FasL and the production of H₂O₂ by macrophages and neutrophils. This example shows that for the first time that the absence of L-arginine may also play a role in the induction of these defects. L-arginine levels are regulated in vivo by three enzymatic pathways in macrophages and endothelial cells, namely iNOS, arginase I and arginase II. Clinical conditions such as severe trauma or following liver transplantation are accompanied by the massive release of large quantities of arginase and the depletion of L-arginine to undetectable levels. This in turn is accompanied by T-cell dysfunction. Arginase it is also produced by several tumor cells and certain strain of bacteria. In addition enterocytes from transgenic mice over-expressing arginase I display poor lymphoid tissue development, although the level of Igs is normal. Therefore, it is possible that the depletion of L-arginine either systemically (trauma and liver transplantation) or in the microenvironment (cancer and infectious diseases) could lead to the decrease in CD3ζ and T-cell dysfunction described in those diseases.

### Example V

This Example Demonstrates That Arginase I Produced by Macrophages, and not Arginase II or Inducible Nitric Oxide Synthase, Modulates T-cell Function and Proliferation.

### Methods and Materials

Tissue Culture Media and Reagents:

RPMI 1640 containing 150 μM L-Arg and without L-Arg (Invitrogen-Gibco, Grand Island, N.Y.) was used to culture Jurkat, T-cells and peritoneal macrophages (PM). The normal physiological concentration of L-Arg in serum ranges between 50-150 μM. Media were supplemented with 4% fetal calf serum (Hyclone, Road Logan, Utah), 25 mM HEPES (Invitrogen-Gibco), 4 mM L-glutamine (Biowhitaker, Walkerville, Md.) and 100 U/mL of Penicillin/Streptomycin (Invitrogen-Gibco). In some experiments, 2 mM L-Arg (Sigma, St Lois, Mo.) and 2 mM L-glutamine (Biowhitaker) were added to co-cultures containing IL-4+IL-13 or IFN-γ stimulated PM and Jurkat cells. Murine rIL-4 (R&D systems), murine rIL-13 (R&D systems) and murine rIFN-γ (R&D systems) concentrations were titrated (data not shown) and used at 50 U/ml, 50 ng/ml and 100 U/ml, respectively. Inhibitors included 50 μM NOHA, an inhibitor of both iNOS and ASE (Calbiochem, San Jose, Calif.), the specific ASE inhibitor Nor-NOHA (100 μM), the NOS inhibitor L-NIL (5 μg/ml) and the hydrogen peroxide scavenger catalase (200 U/ml) (Merck, Indianapolis, Ind.). Analogues of L-Arg L-NMMA (1 mM), L-NNA (1 mM) and L-NAME (1 mM) from Calbiochem were used to test the role of CAT-2B in the modulation of the extra-cellular levels of L-Arg.

Cell Lines and Mice:

Jurkat T-cells, a CD4⁺ cell line (Clone E6-1) (ATCC, Manassas, Va.) and normal stimulated T-cells were used to test the effect of ASE I, ASE II and iNOS produced by PM on CD3ζ expression. Six-week old female C57BL/6 mice were used to isolate peritoneal macrophages (PM). Briefly, 3 ml of thioglycolate-brewer (Becton Dickinson, San Jose, Calif.) were injected into the peritoneal cavity of mice for 3 days. Mice were then sacrificed and PM isolated by washing the peritoneum with HBSS. After 2 days of culture in RPMI with 4% fetal bovine serum, unattached cells were washed off and attached cells were used in co-cultures with Jurkat or normal T-cells.

T-cell Isolation and Antigen Stimulation:

T-cells were isolated from mononuclear cells of healthy donors by T-cell enrichment columns (R&D Systems, Minneapolis, Minn.) according to manufacturer specifications. T-cell purity (CD3+) ranged between 89-95%. The cycle of internalization and re-expression of the TCR-CD3 complex was induced by T-cell stimulation with 1 μg/ml of anti-CD3, 100 ng/ml of anti-CD28 (Becton Dickinson) and 10 μg/ml of goat anti-mouse IgG (KPL, Gaithersburg, Md.). Cells were stimulated in the absence of L-Arg and added to the co-cultures 24 h after stimulation.

Co-cultures in Transwells (Boyden Chambers):

PM were cultured in 6 well plates in RPMI containing 150 μM L-Arg and stimulated with murine rIL-4 (50 U/ml) and murine rIL-13 (50 ng/ml), or murine rIFN-γ (100 U/ml) for 24 hours. Then 1×10⁶ normal T-cells stimulated with anti-CD3 plus anti-CD28 or 1×10⁶ Jurkat cells, were cultured in the top chamber of a transwell system having 0.4 μm pores (Falcon, Franklin lakes, N.J.). CD3ζ expression was measured in Jurkat cells or T lymphocytes at different time points. PM were detached using trypsin/EDTA and the expression of protein and RNA for CAT-2B, ASE I, ASE II and iNOS was tested by western blot and northern blot respectively. Cytoplasmic extracts from PM were used to test ASE activity. Supernatants from stimulated PM were used to measure NO production using the Griess reagent method (Molecular Probes, Eugene, Oreg.). In addition, the extra-cellular concentration of L-Arg was tested in supernatants by HPLC (Dr. Gu Yao Wu laboratory, University of Texas) as described previously. O'Quinn, P. R., D. A. Knabe, and G. Wu. 2002. Arginine catabolism in lactating porcine mammary tissue. J Anim Sci. 80:467-474.

Antibodies and Probes:

Anti-CD3ε-FITC, anti-CD3ζ-PE (Beckman-Coulter, Miami, Fla.) were used for flow cytometry. Mouse IgG1-FITC and mouse IgG1-PE (Beckman-Coulter) were used as isotype controls. Monoclonal antibodies against CD3ζ (Santa Cruz Biotechnologies, Santa Cruz, Calif.), CD3ε (DAKO, Carpinteria, Calif.), iNOS (Santa Cruz Biotechnologies), ASE I (Transduction-Becton Dickinson) and ASE II (a kind gift of Dr. Sidney Morris Jr) were used for western blots. To test the expression of CAT transporters mRNA, specific amplification products from RT-PCR of CAT-1, CAT-2A, CAT-2B and CAT-3 were purified from agarose gels and used as probes to detect CAT expression by northern blot. Murine full-length cDNA for glyceraldehyde-3 phosphate dehydrogenase (GAPDH) (1.6 Kb) (Clontech, Palo Alto, Calif.) was used as housekeeping control.

Flow Cytometry:

Flow cytometry analysis was performed as previously described. Taheri, F., J. B. Ochoa, Z. Faghiri, K. Culotta, H. J. Park, M. S. Lan, A. H. Zea, and A. C. Ochoa. 2001. L-Arginine regulates the expression of the T-cell receptor zeta chain (CD3zeta) in Jurkat cells. Clin Cancer Res 7:958s-965s. Briefly, 5×10⁵ Jurkat cells or T lymphocytes were washed once with Dulbecco phosphate-buffered saline 1× (D-PBS) and resuspended in 200 μL of D-PBS containing 1 μg of anti-CD3ε or isotype control. Cells were incubated for 15 min at 4° C., washed with D-PBS and resuspended in 200 μl D-PBS containing 500 μg/ml of digitonin plus 1 μg of anti-CD3ζ or 1 μg of isotypic control. Cells were incubated for 8 minutes after which they were washed and resuspended in 400 μl of D-PBS. Fluorescence acquisition and analysis were done using a Coulter-EPICS XL flow cytometer (Beckman-Coulter) with a 488 nm argon laser. The data were expressed as mean channel fluorescence intensity (MFI).

Northern Blot:

Two million PM stimulated with IL-4+IL-13 or IFN-γ were used for RNA extraction using lysis with TRIzol (Invitrogen-Gibco) following the manufacturer's specifications. Four micrograms of total RNA from each sample were electrophoresed under denaturing conditions, blotted onto nytran membranes (Schleicher & Schuell Inc, Keene, N.H.) and cross-linked by UV irradiation. Membranes were pre-hybridized at 42° C. in ULTRAhyb buffer (Ambion, Austin, Tex.) and hybridized overnight with 1×10⁶ cpm/mL of ³²P-labeled probe. Probes for detection of CAT transporters and GAPDH mRNA were labeled by random priming using a RediPrime Kit (Amersham, Arlington Heights, Ill.) and (α-³²P) dCTP (3,000 Ci/mmol; NEN Life science products, Boston, Mass.). Membranes were washed and subjected to autoradiography at −70° C. using Kodak Biomax-MR (Eastman Kodak Company) films and intensifying screens.

Extracts and Western-blot Analysis:

Cytoplasmic extracts from PM, Jurkat cells or T lymphocytes were prepared as previously reported. Taheri, F., J. B. Ochoa, Z. Faghiri, K. Culotta, H. J. Park, M. S. Lan, A. H. Zea, and A. C. Ochoa. 2001. L-Arginine regulates the expression of the T-cell receptor zeta chain (CD3zeta) in Jurkat cells. Clin Cancer Res 7:958s-965s. Briefly, cells were resuspended in lysis buffer (50 mM HEPES, 150 mM NaCl, 5 mM EDTA, 1 mM NaOV₄ and 0.5% Triton) containing 50 μg/ml of aprotinin, 50 μg/ml of leupeptin, 100 μg/ml of trypsin-chymotrypsin inhibitor and 2 mM PMSF. Lysates were centrifuged at 3000×g for 10 min at 4° C. Cytoplasmic extracts from Jurkat or T-cells were immunoblotted for CD3ζ and CD3ε. The expression ASE I, ASE II and iNOS was detected by immunoblot using PM extracts. GAPDH was used as housekeeping protein. Cytoplasmic extracts were electrophoresed in 12 or 8% Tris-Glycine gels (Novex, San Diego, Calif.), transferred to PVDF membranes and immunoblotted with the appropriate antibodies. The reactions were detected using the ECL kit (Amersham-Pharmacia).

ASE Activity Assay:

Cell lysates from PM stimulated with IL-4+IL-13 or IFN-γ were tested for ASE activity by measuring the production of L-ornithine. In brief, cell lysates from PM stimulated with IL-4+IL-13 or IFN-γ in the presence or absence of NOHA or Nor-NOHA were added to 50 μl of Tris-HCl (50 mM; pH 7.5) containing 10 mM MnCl₂. This mixture was heated at 55-60° C. for 10 min to activate ASE. The hydrolysis reaction from L-Arg to L-ornithine was identified by a colorimetric assay after the addition of ninhydrin solution and incubation at 37° C. for 1 h.

### Results for This Example

Peritoneal Macrophages Stimulated With IL-4+IL-13 Deplete Extra-cellular L-Arg:

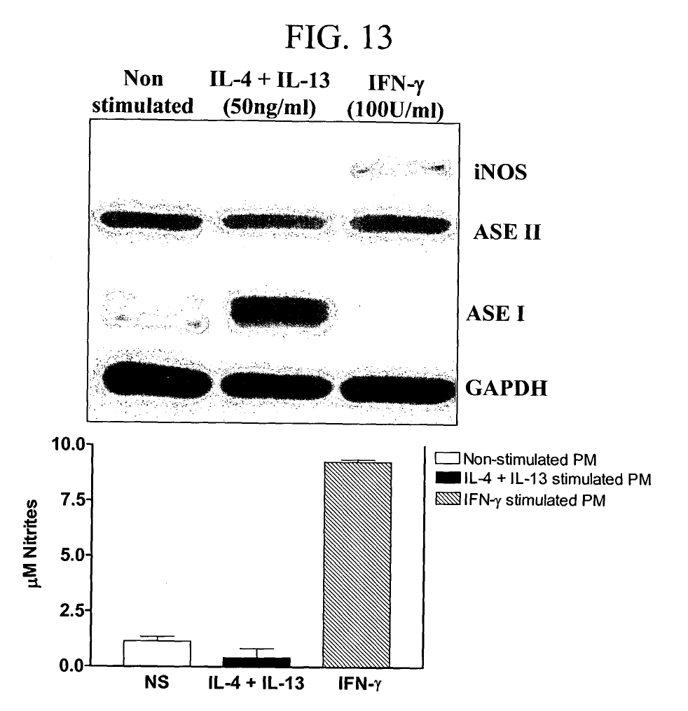

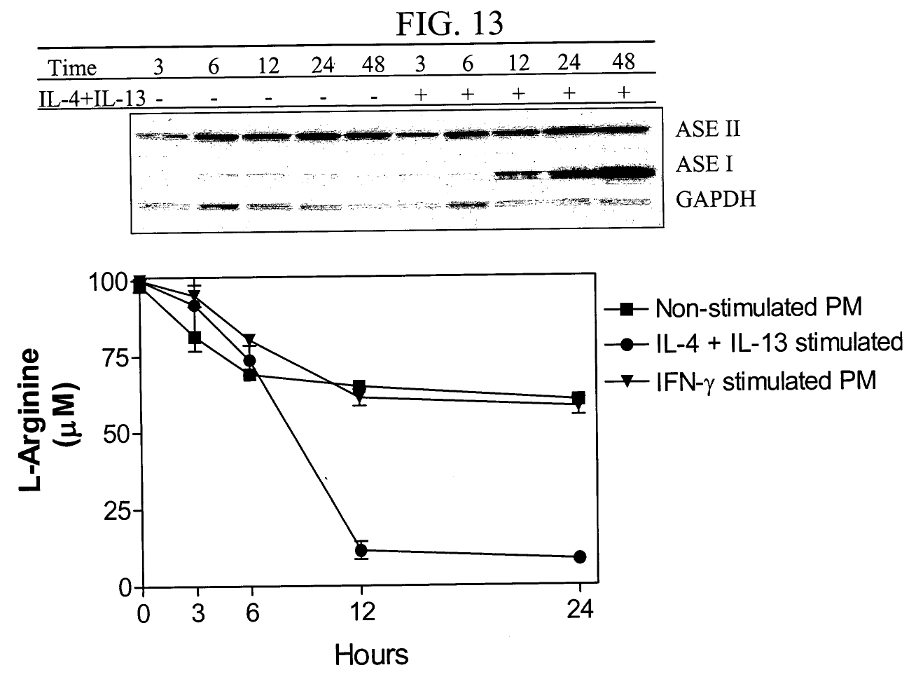

Previous reports have shown that the expression of iNOS and ASE I in murine macrophages is differentially regulated by Th1 and Th2 cytokines. As shown in FIG. 13A, peritoneal macrophages (PM) stimulated with IL-4+IL-13 up-regulate ASE I but not ASE II or iNOS. In contrast, PM stimulated with IFN-γ increase iNOS expression and NO production (as measured by the production of nitrites), and decrease ASE I expression (FIG. 13A, B). IL-4+IL-13 stimulation increases ASE I mRNA expression in the first 4 hours (data not shown), with an increase in ASE I protein by 12 hours, while the mitochondrial isoform ASE II remains unchanged during the same time (FIG. 13C). Activation of PM with these Th1 or Th2 cytokines also had different effects on extra-cellular levels of L-Arg. PM stimulated with IL-4+IL-13 rapidly reduced L-Arg in the tissue culture medium to levels below 15 μM in the first 12 hours of culture (FIG. 13D). In contrast, PM stimulated with IFN-γ only produced a moderate decrease in L-Arg (P<0.005), which was similar to unstimulated PM.

ASE I Depletes L-Arg and Induces CD3ζ Down-regulation in T-cells:

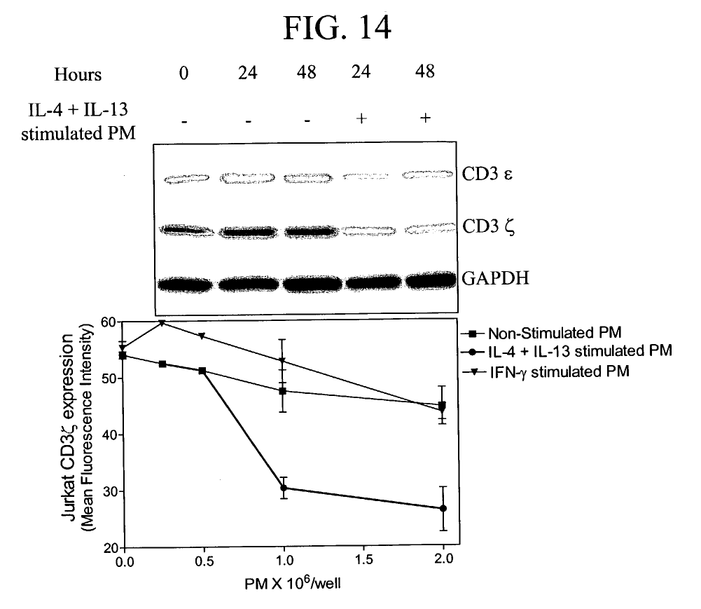

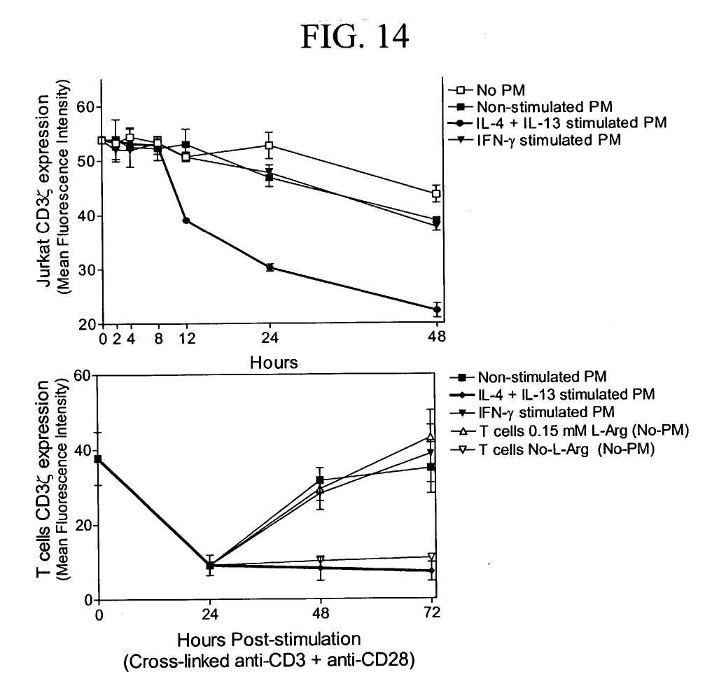

Although the present inventors previously published that Jurkat T-cells cultured in the absence of L-Arg have a decreased expression of CD3ζ within 12-24 hours, it was unclear whether the gradual decrease of L-Arg by ASE I expressing macrophages would have an impact on T-cells. Using a transwell system, macrophages stimulated with IL-4+IL-13 or IFN-γ were cultured in the bottom chamber and Jurkat cells (as marker for L-Arg depletion) in the top chamber, separated by a 0.4 μm pore filter. PM stimulated with IL-4+IL-13 induced a rapid decrease in the expression of CD3ζ and CD3ε in co-cultured Jurkat cells (FIG. 14A), which coincided with the increase in ASE I expression (FIG. 13C) and the reduction in the extra-cellular levels of L-Arg (FIG. 13D). In contrast, PM stimulated with IFN-γ or unstimulated PM did not alter CD3ζ or CD3ε expression. This effect was dependent on the number of PM and the time in culture (FIG. 14B, C). Control Jurkat cells (without PM) cultured with IL-4+IL-13 or IFN-γ did not show changes in CD3ζ expression (data not shown).

Normal T lymphocytes cultured in the absence of L-Arg also loose CD3ζ expression, however through different mechanisms from those seen in Jurkat cells. The absence of L-Arg does not modulate CD3ζ expression in resting T-cells. Instead, lack of L-Arg alters the cycle of internalization and re-expression of the TCR-CD3 complex following antigen stimulation, by preventing CD3ζ re-expression. T-cells stimulated with anti-CD3+anti-CD28 and co-cultured in transwells with PM activated with IL-4+IL-13 showed a persistent decrease in the expression of CD3ζ, similar to T-cells stimulated and cultured in medium without L-Arg (FIG. 14D). In contrast, T-cells co-cultured with resting PM or PM stimulated with IFN-γ displayed the normal cycle of internalization and re-expression of CD3ζ within 48 hours, as did control T-cells cultured in RPMI containing 150 μM L-Arg (FIG. 14D) or T-cells cultured with IL-4+IL-13 or IFN-γ (data not shown). Similar results were obtained using autologous murine T-cells co-cultured with IL4+IL13 stimulated PM (data not shown).

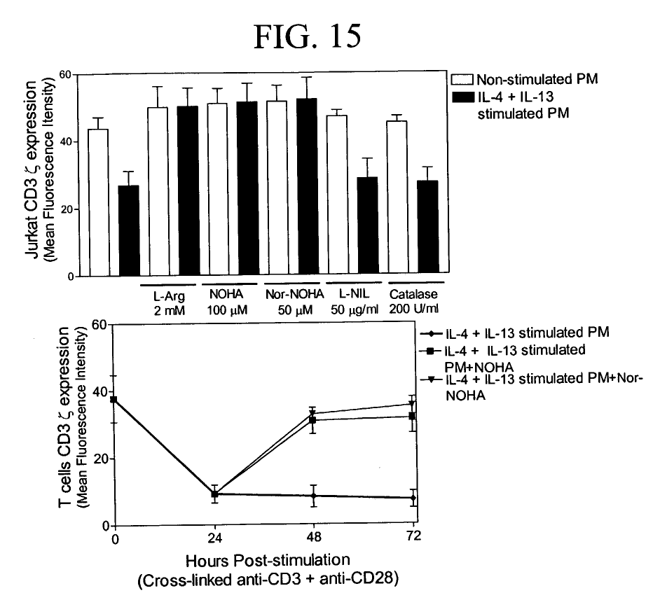

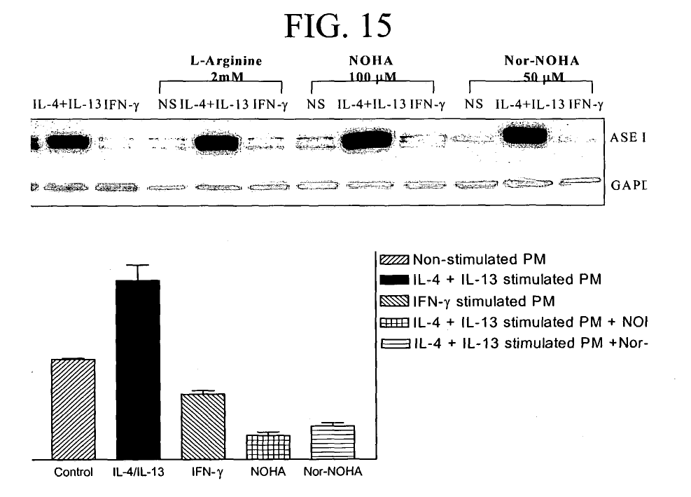

Inhibitors of ASE and iNOS were then used to further determine the role of these enzymes in the regulation of extra-cellular L-Arg and the expression of CD3ζ. The presence of ASE inhibitors NOHA or Nor-NOHA in the cultures or the addition of excess of L-Arg (2 mM) prevented the decrease of CD3ζ in Jurkat cells induced by co-culture with PM stimulated with IL-4+IL-13 (FIG. 15A) and partially blocked the rapid decrease in the extra-cellular levels of L-Arg (Table 6). In contrast, the iNOS inhibitor L-NIL did not prevent CD3ζ down-regulation. Catalase, a hydrogen peroxide scavenger, was included in some of the cultures since hydrogen peroxide produced by activated macrophages and neutrophils has also been shown to induce a decrease in CD3ζ expression. Catalase however did not prevent the decrease of CD3ζ induced by IL-4+IL-13 stimulated PM, demonstrating that hydrogen peroxide was not the cause for the loss of CD3ζ in this model. The addition of ASE inhibitors NOHA and Nor-NOHA also prevented the loss of CD3ζ in normal stimulated T lymphocytes co-cultured with IL-4+IL-13 activated PM (FIG. 15B). NOHA and Nor-NOHA did not prevent ASE I up regulation after IL-4+IL-13 stimulation (FIG. 15C), but instead blocked intracellular ASE activity (FIG. 15D).

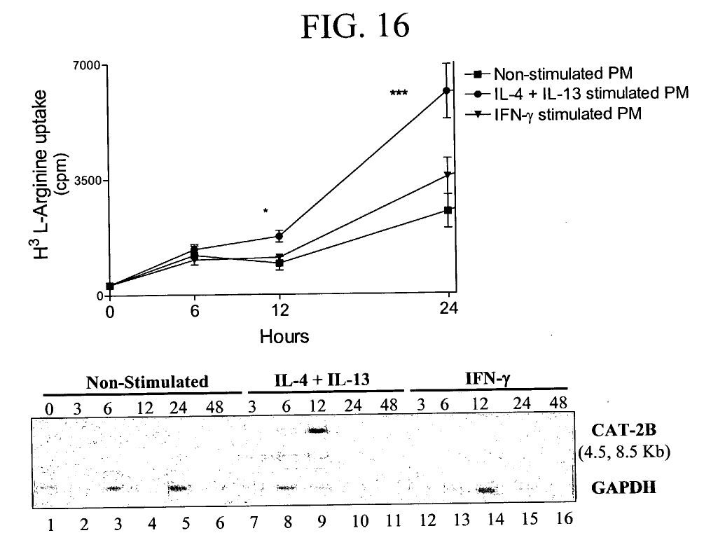

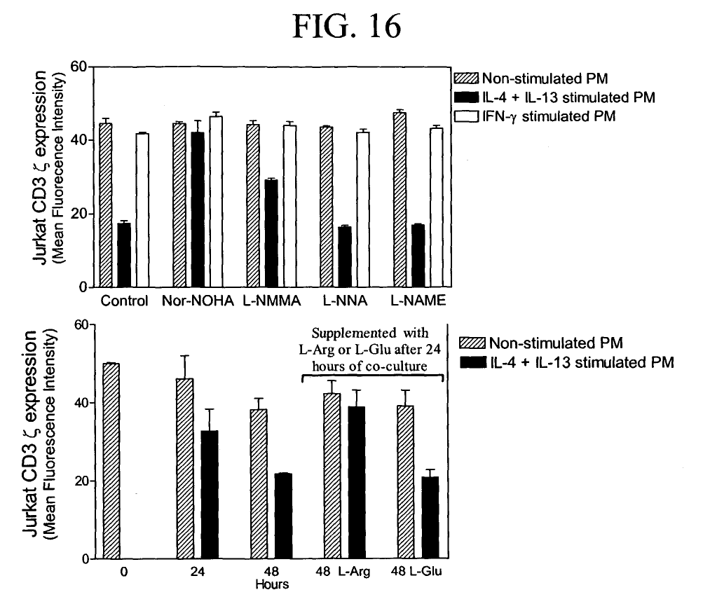

ASE I/CAT-2B increase L-Arg uptake in macrophages: Low levels of serum L-Arg, following liver transplantation, is caused by a significant and rapid increase of ASE in circulation. Therefore, the decrease in L-Arg levels induced by IL-4+IL-13 stimulated PM could be the result of ASE I released into the tissue culture media. Interestingly, there was no a significant ASE activity in the supernatants from IL-4+IL-13 stimulated PM (data not shown). Therefore, this example tested whether L-Arg uptake by PM could in part explain the depletion of extra-cellular L-Arg. L-Arg uptake was significantly higher at 12 and 24 hours in PM stimulated with IL-4+IL-13 than in PM stimulated with IFN-γ or non-stimulated PM (P<0.005) (FIG. 16A). L-Arg is transported into cells by the recently described cationic amino acid transporter Y⁺ family of receptors (CAT). The increase in the H³-L-Arg uptake and the decreased extra-cellular levels of L-Arg was paralleled by an increased CAT-2B mRNA expression in IL-4+IL-13 stimulated PM (FIG. 16B). In contrast, there was no increase in CAT-2B mRNA in PM stimulated with IFN-γ. Furthermore, the L-Arg analogue L-mono-methyl-L-Arg (NMMA), which competitively inhibits CAT-2B, partially prevented the CD3ζ down-regulation induced by IL-4+IL-13 stimulated PM (FIG. 16C). Other L-Arg analogues, not transported by CAT carriers such as N-NO2-L-Arg (L-NNA) and N-NO2-L-Arg-OMe (L-NAME), did not prevent CD3ζ decrease. Stimulation with IL-4+IL-13 or IFN-γ did not induce significant changes in the expression of other CAT transporters including CAT-1, CAT-2A and CAT-3 (data not shown). Finally, if the ASE I/CAT-2B pathway is an important mechanism in the depletion of extra-cellular L-Arg and CD3ζ down-regulation, then addition of excess exogenous L-Arg should saturate this pathway and reverse the decrease in CD3ζ. As shown in FIG. 16D, addition of 2 mM L-Arg but not L-glutamine induced the re-expression of CD3ζ. Furthermore, the addition of excess of L-ornithine or urea did not induce CD3ζ down-regulation, suggesting that L-Arg consumption instead of L-ornithine or urea production leads to decrease on CD3ζ expression (data not shown).

In conclusion these results suggest that IL-4+IL-13 stimulation of PM causes the up-regulation of ASE I/CAT-2B pathway, which increases the incorporation and metabolism of L-Arg resulting in the reduction of extra-cellular levels of L-Arg. The decrease in L-Arg in turn leads to the low expression of CD3ζ in Jurkat and normal stimulated T-cells.

### Discussion of Results

L-Arg is a non-essential amino acid that plays a central role in several biological systems including the immune response. In macrophages, L-Arg is metabolized by ASE I, ASE II and the nitric oxide synthase family (NOS) of enzymes. ASE I and ASE II, encoded by two distinct genes, hydrolyze L-Arg into urea and L-ornithine, the latter of which is the main substrate for the production of polyamines (putrescine, spermidine and spermine), required for cell cycle progression. L-Arg is also metabolized in macrophages by iNOS to citrulline and NO, a highly reactive compound important in the cytotoxic mechanism of these cells. The importance of L-Arg on the immune system has been demonstrated by the significant decrease in NO production caused by the depletion of circulating L-Arg in patients and rodents following liver transplantation, trauma or sepsis. Furthermore, trauma patients display a poor T-cell response, which recovers with the enteral supplement of L-Arg, suggesting a possible role this amino acid in modulating T-cell function. Other diseases including cancer and certain chronic infections such as tuberculosis and leprosy are also characterized by T-cell dysfunction, which may in part be explained by the decreased expression of T-cell signal transduction proteins including CD3ζ. The inventors have shown that T-cells loose CD3ζ expression when cultured in low concentrations of L-Arg. Thus, it was asked whether macrophages could modulate the availability of L-Arg and alter the expression of CD3ζ chain in Jurkat cells (as marker of L-Arg reduction) and normal T lymphocytes.

The data discussed herein confirmed previous observations demonstrating the reciprocal regulation of iNOS and ASE I in macrophages by Th1 and Th2 cytokines, for example Th1 cytokines such as IFN-γ increased iNOS, while Th2 cytokines, IL-4 plus IL-13, enhanced ASE I. The expression of ASE II does not appear be regulated by Th1 or Th2 cytokines. An increased production of Th2 cytokines has been frequently described in chronic intracellular infections such as leishmaniasis, leprosy and in some cancers, which could therefore cause an increased ASE I expression and possibly T-cell dysfunction.

The data shown herein demonstrate that surprisingly, ASE I, but not iNOS and ASE II produced in PM rapidly decreases extra-cellular L-Arg concentrations to levels below 10 μM. Chang et al (Cancer Res. 61:1100-1106.) and Que et al (Am.J Clin.Nutr. 76:128-140.) had previously demonstrated a significant decrease in the extra-cellular levels of L-Arg in an in vitro model using ASE I transfected cell lines. Furthermore, transgenic mice having enterocytes that over-express ASE I have a selective decrease of L-Arg in serum. In addition to ASE I, the coordinated expression of CAT proteins that transport L-Arg from extra-cellular microenvironment into the cell also play an important role in the regulation of extra-cellular levels of L-Arg. This particular carrier system is characterized by its high affinity for basic amino acids, its independence of Na⁺ and the ability of substrate on the opposite (trans) side of the membrane to increase transport activity. CAT genes have been recently cloned and designated CAT-1, CAT-2A, CAT-2B, and CAT-3. Whereas CAT-1, CAT-2B, and CAT-3 are high-affinity (Km 100 μmol/L) transporters for L-Arg, CAT-2A is an alternative splice variant of CAT-2B that possesses low affinity for L-Arg (Km 1 to 2 mmol/L). In accordance with Louis et al, the data in this example show that PM stimulated with IL-4+IL-13 up-regulate the expression of CAT-2B displaying similar kinetics to ASE I (FIG. 13C, 16B). Instead, IL-4+IL-13 did not induce major changes in the expression of CAT-1 and CAT-2A in PM (data not shown).

In vitro data show that L-Arg concentrations below 40 μM cause the rapid decrease of CD3ζ chain in Jurkat cells and impair the re-expression of this chain in stimulated T-cells. Several reports have demonstrated that rodents and patients with trauma or undergoing liver transplantation have a rapid decrease in circulating L-Arg levels to concentrations below 40 μM. It is also possible that L-Arg levels at sites of tumors may be more reduced than in circulation, which might explain the preferential decrease in CD3ζ chain in tumor infiltrating T-cells. However, this phenomenon is readily reversible by culturing these cells in vitro, which can occur because these T-cells are cultured in CRPMI that contains 1140 μM L-Arg.

The molecular mechanisms involved in the control of gene expression by amino acid deprivation have been extensively studied in yeast. However, the effect of starvation of different amino acids in mammalian cells is less clear. Recent publications have suggested a close correlation between amino acid availability and immune response. Munn et al described that tryptophan metabolism by macrophages producing indoleamine 2,3-dioxygenase inhibit T-cell proliferation. This group also suggested that tryptophan starvation induced cell cycle arrest in normal T lymphocytes and sensitizes activated T-cells to apoptosis prior to cell division. The absence of the amino acid leucine have also been associated with an increase in the amount and stability of mRNA for the CHOP gene. This gene encodes a transcription factor that interacts with CCAAT/enhancer-binding proteins family, which in turn inhibits the normal proliferation of cells. Therefore, essential amino acids appear to induce changes in T-cells that ultimately inhibit their normal proliferation. Our data show that metabolism of the non-essential amino acid L-Arg can control T-cell function through modulation of CD3ζ, which appears to be the result of a decreased CD3ζ mRNA stability. Furthermore, L-Arg starvation induces in Jurkat cells a de novo protein that releases a ribonucleoprotein complex bound to the 3′ UTR of CD3ζ mRNA, reducing its stability. L-Arg starvation also altered CD3ζ re-expression in normal T-cells after anti-CD3 plus anti-CD28 stimulation, which was caused by a specific decrease in CD3ζ.

ASE I is produced by several tumors including gastric, colon, breast and lung cancers. Most reports have associated the increased ASE expression by tumor cells with the need to produce polyamines that sustain rapid tumor proliferation. It is also possible, as shown by our data, that the increase in ASE I expression and the consequent reduction of L-Arg may have as a secondary effect the decreased expression of CD3ζ and the inhibition of T-cell function. Preliminary data in animal models indeed suggest that ASE I production in tumor bearing mice by specific populations inside the tumor can modulate CD3ζ expression in T-cells. ASE I is not only expressed by tumor cells but also by several bacteria and parasites. Therefore it is possible that the mechanisms presented here could be used by certain pathogens as a common mechanism to induce T-cell dysfunction in these diseases.

A decreased expression of CD3ζ and a diminished T-cell function have been repeatedly reported in patients with cancer, chronic infections (leprosy) and autoimmune diseases such as lupus. The lack of a mechanism that could explain the loss of this important T-cell signal transduction molecule in diseases as different as cancer and infectious diseases has made these findings controversial. Studies in cancer suggest that Fas-Fas ligand interaction between tumor cells and T lymphocytes can lead to an increased T-cell apoptosis and the loss of CD3ζ chain. Other investigators have shown that the production of hydrogen peroxide by macrophages and neutrophils in cancer patients may be responsible in part for the loss of CD3ζ. Kono, et al. Eur J Immunol 26:1308-1313; Otsuji, et al. Proc.Natl.Acad.Sci.U.S.A 93:13119-13124; and Ochoa et al. Ann.Surg. 214:621-626.Here, it is suggested that the regulation of the L-Arg availability by ASE I/CAT2B pathway in macrophages may play a role in the induction of these T-cell alterations.

In summary, these results present a novel mechanism by which macrophages, dendritic cells, some microorganisms and certain tumors may regulate the availability of L-Arg in the microenvironment inducing alterations in the expression of some T-cell signal transduction proteins and ultimately causing T-cell dysfunction.

TABLE 6
Arginase inhibitors prevent the depletion of extra-cellular
L-Arg levels induced by PM stimulated with IL-4 + IL-13

| | |
|---|---|
| Non-stimulated | IL-4 + IL-13 |

| | | | | |
|---|---|---|---|---|
| PM | 64.9 | (6.2)* | 7.8 | (49) |
| PM + Exogenous | 1267 | (123.6) | 59.8 | (11.4) |
| L-Arg (2 mM) | | | | |
| PM + NOHA | 101.6 | (9.6) | 75.6 | (13.1) |
| (100 μM) | | | | |
| PM + Nor-NOHA | 109.8 | (8.3) | 73.8 | (18.4) |
| (50 μM) | | | | |

*Mean of μM L-Arg (±SD) in three different experiments

### Example VI

This Example Demonstrates That Helicobacter pylori Arginase Inhibits T-cell Proliferation and Reduces T-cell Function.

### Introduction

Helicobacter pylori (H. pylori) infection has been associated with diseases ranging from gastritis to gastric cancer and mucosa-associated lymphoid tissues (MALT) lymphomas. Asaka, M., et al. J. Gastroenterol. 29 Suppl. 7, pp.100-104 (1994); Du, M. et al. Lancet Oncol. 3, pp. 97-104 (2002); Hori, K., et al. J. Gastroenterol. 37, pp. 288-292 (2002). Low income, over-crowding and other factors characteristic of lower socioeconomic status are related to the high prevalence of the infection. Malaty, H. M., et al. Gut 35, pp. 742-745 (1994); Malaty, H. M., et al. Helicobacter. 1, pp. 82-87 (1996); Torres, J. Rev. Gastroenterol. Mex. 65, pp. 13-19 (2000). All infected individuals present histological signs of gastritis, Annibale, B., et al. Helicobacter. 6, pp. 225-233 (2001); Loffeld, R. J. Neth. J. Med. 54, pp. 96-100 (1999), but many do not develop clinical symptoms of the disease. Cave, D. R. Semin. Gastrointest. Dis. 12, pp. 196-202 (2001); Joshi, A., et al. Trop. Gastroenterol. 22, pp. 194-196 (2001); Strauss, R. M., et al. Am. J. Med. 89, pp. 464-469 (1990). Gastric pathology appears to be closely associated with H. pylori virulence genes and with the immune response of the infected host against the bacterium. Glupczynski, Y. et al. Eur. J. Gastroenterol.Hepatol. 9, pp. 447-450 (1997); Kidd, M., et al. Gut 45, pp. 499-502 (1999); Nogueira, C., et al. Am. J. Pathol. 158, pp. 647-654 (2001); Telford, J. L., et al. Curr. Opin. Immunol. 9, pp. 498-503 (1997). In murine models a Th1 response is associated with damage to the mucosa, while a Th2 response appears to be protective. Nedrud, J. G., et al. Basic mechanisms and clinical cure, pp. 101-109 (1998). In addition, IRF-1 knockout mice that are unable to establish a Th1 response, do not develop damage of the gastric mucosa after exposure to H. pylori. Sommer, F., et al. Eur. J. Immunol. 31, pp. 396-402 (2001).

In humans, a Th1 response is observed in some patients with active gastritis and duodenal ulcer, however, this response does not appear to confer protection against H. pylori. Blanchard, T. G., et al. Curr. Top. Microbiol. Immunol. 241, pp. 181-213 (1999); Ernst, P. B. et al. Acta Odontol.Scand. 59, pp. 216-221 (2001); Ernst, P. B., et al. Dig. Dis. 19, pp. 104-111 (2001). Therefore it is possible that H. pylori, like other bacteria, has mechanisms to escape the immune response. Virulent strains of H. pylori carrying the cag pathogenicity island (PAI), delay phagocytosis by macrophages in vitro and are killed less efficiently than those without the PAI element. Allen, L. A., et al. J. Exp. Med. 191, pp. 115-128 (2000); Ramarao, N., et al. Mol. Microbiol. 37, pp. 1389-1404 (2000); Ramarao, N., et al. Infect. Immun. 69, pp. 2604-2611 (2001). Recent reports have also shown the CagA binds to SHP-2 phosphatase, which could impair signal transduction in cells. Higashi, H., et al. Science 295, pp. 683-686 (2002).

L-Arginine is a key factor in the activation and function of T-cells. Its depletion results in T-cell dysfunction. Taheri, F., et al. Clin. Cancer Res. 7, pp. 958s-965s (2001). The possibility that H. pylori arginase could modulate T-cell function and limit L-arginine availability was therefore studied. To survive the stomach environment, H. pylori produces arginase that hydrolyzes L-arginine to urea and ornithine. Urea is then converted by urease to ammonia that neutralizes the acidic pH in the stomach. Jurkat T-cells and normal T lymphocytes cultured in the presence of an H. pylori sonicate or co-cultured with H. pylori in a transwell system (Boyden Chamber), showed a significant decrease in proliferation, which was paralleled by a reduced expression of CD3ζ chain of the T-cell receptor. Pre-incubation of the H. pylori sonicate with N-hydroxy-L-arginine (NOHA), an arginase inhibitor, or with excess L-arginine prevented the loss of the CD3ζ chain and maintained the ability of T-cells to proliferate. Furthermore, co-culture of Jurkat cells with live isogenic strains of H. pylori using a trans-well system showed that arginase produced by the wild type H. pylori strain but not by the mutant strain, rocF(−), decreased CD3ζ and blocked T-cell proliferation.

### Materials and Methods

Helicobacter pylori Sonica:

H. pylori ATCC 43504 strain was cultured on blood agar (Becton-Dickinson Microbiology System; Sparks, Md.) for 5 days using the BBL Campy Pouch System (Becton-Dickinson) to generate a microaerobic environment. After 5 days the cells were collected and washed twice in PBS. The bacteria were resuspended in PBS and subjected to six rounds of 30 s sonication at 200 W using a Cell Disruptor Model 450 (Branson Sonifier; Eagle Road, Conn.). The sonicated bacteria were centrifuged at 20,000×g for 30 min at 4° C. and the supernatant was collected and filtered through a 0.2 μm filter. The protein concentration of the supernatant was determined by BCA assay, following manufacturer's instructions (Pierce; Rockford, Ill.).

H. pylori Arginase Mutant:

An H. pylori ATCC 43504 arginase mutant rocF(−) was created by transforming wild type 43504 with the rocF disruption plasmid, pBS-rocF::aphA3. McGee, D. J., et al. J. Bacteriol. 181, pp. 7314-7322 (1999). A kanamycin resistant transformant was confirmed by PCR as described before. McGee, D. J., et al. J. Bacteriol. 181, pp. 7314-7322 (1999). Bacterial sonicates were prepared as described above and arginase activity measured as described later.

Proliferation Assays:

Unless otherwise stated, the cells were always cultured in RPMI 1640 (Gibco Life-Technologies; Rockville, Md.) which contains 1104 μM L-arginine, 2.5 mM of L-glutamine, (Gibco), 25 mM HEPES (Gibco), 100 μg/ml of penicillin/streptomycin (Gibco), and 10% heat inactivated FCS (Hyclone). Jurkat cells were maintained at 0.5×10⁶/ml. For proliferation assays Jurkat cells or peripheral blood mononuclear cells (PBMC) were placed at 2×10⁵/well in round-bottom 96-well plates (Corning; Corning, N.Y.) and incubated for 2 h with increasing concentrations of the H. pylori sonicate ranging from 0.01 μg/ml to 100 μg/ml. After this, 30 ng/ml of anti-CD3 (Ortho Diagnostics, Raritan, N.J.) and 100 ng/ml of anti-CD28 (Becton-Dickinson Biosciences; Palo Alto, Calif.) were added to the PBMC. Jurkat cells were incubated for an additional 24 h and the PBMC for 48 h. One μCi of ³H-thymidine (NEN Life Sciences Products; Boston, Mass.) was added per well for the last 20 hours. The cells were collected onto glass fiber filters (Unifilter GF/B; Packard Bioscience; Meriden, Conn.) and radioactivity was counted in a Beta counter (Microplate Scintillation and Luminiscence Counter, TopCount, Packard).

CD3ζ Chain Expression:

Jurkat cells were cultured for 24 h in medium with the H. pylori sonicate at concentrations ranging from 1 μg/ml to 50 μg/ml. CagA (2 μg/ml) (Austral Biologicals; San Ramon, Calif.), VacA (4 μg/ml) (Austral), H. pylori urease A (2 μg/ml) (Austral), H. pylori urease B (2 μg/ml) (Austral) and E. coli LPS (500 ng/ml) (Sigma-Aldrich; St Louis, Mo.) were also used. Jurkat and T-cells were counted and stained with FITC-conjugated anti-human CD3 (Beckman-Coulter; Miami, Fla.) for 20 min in the dark. After washing with PBS the cells were stained for 8 min with phycoerythrin (PE)-conjugated anti-human CD3ζ (Beckman-Coulter; Miami, Fla.) in PBS containing 50 μg/ml of digitonin. The cells were washed twice in PBS and analyzed in an EPICS XL Coulter Flow Cytometer. The results compare the percentage of cells expressing CD3ζ as well as the mean fluorescence intensity.

JAM Cytotoxicity Assay:

Jurkat cells were radioactively labeled by culturing in RPMI containing 20 μCi/ml of ³H-thymidine (NEN) for 20 h at 37° C. The cells were then washed, resuspended in twice the initial volume of RPMI (to a concentration of approximately 1×10⁶ cells/ml) and plated in round-bottom 96-well plates in a final volume of 200 μl. The H. pylori sonicate was added at 50 μg/ml and the cells were incubated for an additional 20 h. The cells were collected onto filters and the radioactivity was counted. The results are expressed as percent radioactivity of the control cells cultured without the H. pylori sonicate.

Apoptosis:

Jurkat cells were treated with the H. pylori sonicate for 24 h and stained with the non-isotopic stain Annexin V as recommended by the manufacturer (Oncogene Research Products; La Jolla, Calif.). Briefly, 5×10⁵ Jurkat cells were washed once in 0.5 ml of binding buffer (supplied by the manufacturer) and resuspended in 0.5 ml of binding buffer containing 1.25 μl of annexin V. The cells were incubated for 15 min at room temperature (RT) in the dark, centrifuged at 1000 rpm and 10 μl of propidium iodide (PI) (supplied by the manufacturer) were added per sample. Fluorescence was measured by using an EPICS XL Coulter Flow Cytometer. The percentage of cells undergoing either necrosis (PI positive cells) or apoptosis (Annexin V positive) was determined.

Protein Tyrosine Phosphorylation:

Three million Jurkat cells were cultured for 4 h and 8 h in the presence of 50 μg/ml of the H. pylori sonicate after which the cells were recovered, washed with cold PBS and lysed in a buffer containing 50 mM HEPES, 150 mM NaCl, 5 mM EDTA, 100 mM Na₃VO₄ and 0.5% Triton X-100, pH 7.5. In addition the buffer contained 100 μg/ml aprotinin, leupeptin (Boehringer Manheim, Indianapolis, Ind.), 100 μg/ml trypsin-chemotrypsin inhibitor and 2 mM PMSF (Sigma, St. Louis, Mo.). Proteins were separated by SDS-PAGE, transferred to Immobilon-P membranes (Millipore, Bedford, Mass.) and immunobloted with anti-phosphotyrosine 4G10 antibody (Upstate Biotechnology Inc. Lake Placid, N.Y.). Protein bands were visualized by using enhanced chemioluminiscence (ECL; Amersham-Pharmacia Biotech; Piscataway, N.J.) and X-OMAT AR films (Eastman Kodak Co. Rochester, N.Y.).

L-Arginine and NOHA:

The H. pylori sonicate was pre-incubated overnight with the arginase inhibitor N-hydroxy L-arginine (NOHA) (Sigma-Aldrich) at 100 μg/ml or L-arginine at 2 mM and added to the cultures at a final concentration of 50 μg/ml. After addition of the sonicate plus NOHA or sonicate plus L-arginine mixture to the Jurkat cells, 1.0 μCi of ³H-thymidine (NEN) was added per well, the cells were incubated at 37° C. for 18 h, harvested onto filters and counted in a β-counter (Packard).

Arginase Activity in H. pylori:

To determine arginase activity in the H. pylori sonicate a modified version of the methodology described previously, Mendz, G. L., et al. Biochim. Biophys. Acta 1388, pp. 465-477 (1998), which measures the conversion of L-arginine to L-ornithine was used. Briefly, 25 μl of the H. pylori sonicate were mixed with 25 μl of 5 mM CoCl₂ and incubated at 56° C. for 20 min to activate the enzyme. One hundred and fifty (150) μl of pre-warmed 100 mM Tris (pH 7.4) containing 5 mM L-arginine (Sigma-Aldrich) was added to the mixture. The sample was incubated at 37° C. for 1 h. The reaction was stopped by adding 750 μl glacial acetic acid. Two hundred and fifty (250) μl of ninhydrin solution (2.5 g ninhydrin dissolved in 40 ml of 6M phosphoric acid and 60 ml glacial acetic acid) were added to the sample and heated at 90° C. for 1 h. After cooling, 200 μl of the mixture was plated on flat-bottomed 96 well plates and the absorbance was read at 515 nm using a Benchmark Plus Microplate Spectrophotometer (BioRad; Hercules, Calif.). The concentration of the L-ornithine present on the sample was estimated by using a standard L-ornithine curve ranging from 2 nmoles to 250 nmoles.

L-Arginine Detection by HPLC-ECD:

HPLC-ECD was performed as previously reported, Tcherkas, Y. V., et al. J. Chromatogr. A 913, pp. 303-308 (2001), using an ESA-CoulArray Model 540 (ESA Inc; Chelmsford, Mass.) with an 80×3.2 Column with 120A pore size. Briefly, supernatants were deproteinized by methanol. After centrifugation at 6000×g for 10 min at 4° C., the supernatant was derivatized with 0.2 M OPA/BME (o-phtaldialdehyde containing β-mercaptoethanol). Fifty microliters of the sample were injected per sample. The retention time for L-arginine was 10.2 min. Standars of L-arginine were prepared in methanol.

Purified T-cell Cultures:

After isolating peripheral blood mononuclear cells by Ficoll-Paque Plus (Amersham Pharmacia Biotech; Piscataway, N.J.), T-cells were purified by negative selection by using an affinity column (R & D Systems; Minneapolis, Minn.). The purity was always >90% T-cells as measured by the expression of CD3ε. H. pylori extracts or live H. pylori have no effect on resting T-cells, therefore T-cells were stimulated by cross linking anti-CD3 plus anti-CD28 as follows: 24 well plates were coated with 0.3 ml of goat-anti mouse (GAM) (Kirkegaard & Perry Laboratories; Gaithersburg, Md.) at 10 μg/ml in HBSS for 1 h. After washing twice with Hank's balanced salt solution (HBSS), 0.5×10⁶ of purified T-cells were added per well in 1 ml of RPMI without L-arginine containing 1 μg/ml of anti-human CD3 (Ortho Diagnostics, Raritan, N.J.) and 100 ng/ml of anti-human CD28 (Becton-Dickinson) and cultured for 24 h. After this time L-arginine was added to the wells to give a final concentration of 0.4 mM. H. pylori sonicate (20 μg/ml) from either the wild type strain ATCC 43504 or its rocF (−) isogenic mutant was also added to the wells. After 24 h under these conditions, the cells were recovered and stained for CD3ζ. Six different donors were tested. Using the same system, purified T-cells were cultured with recombinant H. pylori antigens at the concentrations mentioned before and stained for the expression of CD3ζ.

Co-culture of Live H. pylori With Jurkat Cells:

Jurkat cells (0.5×10⁶ per well) were plated in 0.7 ml of RPMI without L-arginine. Live Helicobacter pylori was plated in a transwell insert (0.4 μm pore size, Becton-Dickinson) in RPMI containing 400 μM of L-arginine to give a 400:1 bacteria to cell ratio. The cells were then cultured under microaerobic conditions by using the Campy-Pak system (Becton-Dickinson), at 37° C. for 24 h. The cells were recovered and stained for CD3ζ as well as for Annexin V as described previously.

Statistical Analysis:

Differences between groups were determined by using either paired or unpaired Students' t test. All the statistical analysis was done with GraphPad Prism 3.0 (Graph Pad Software; San Diego, Calif.).

### Results of This Example

H. pylori Sonicate Decreases T-cell Proliferation Without Increasing Apoptosis:

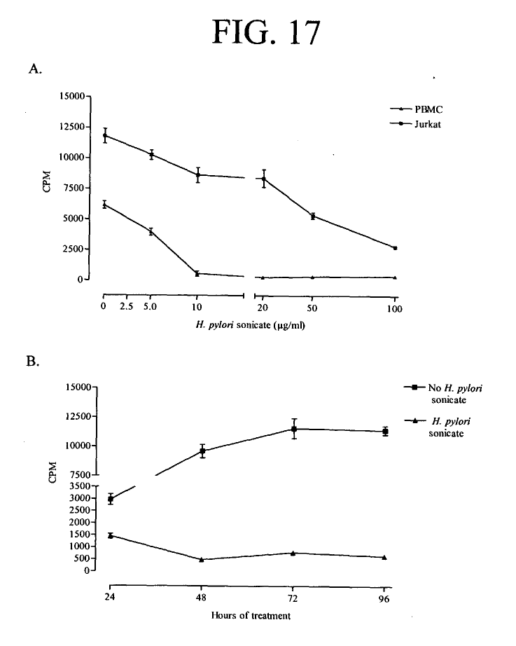

Jurkat cells and antibody-stimulated peripheral blood mononuclear cells (PBMC) were cultured with increasing concentrations of a sonicate derived from the ATCC 43504 strain of H. pylori. As shown in FIG. 17A, there was a dose-dependent decrease in the proliferation of Jurkat cells and activated PBMC. The antibody-stimulated PBMC appeared to be more sensitive to the effects of the H. pylori sonicate as shown by the fact that concentrations as low as 10 μg/ml caused a greater than 90% decrease in proliferation. This effect was not reversed by time in culture as demonstrated in FIG. 17B, where Jurkat cells cultured for up to 96 h in the presence of the H. pylori sonicate, failed to recover their proliferative activity. Jurkat cells were used because under specific culture conditions they have been shown to have a reduction in proliferation and CD3ζ expression similar to anergic T-cells. Taheri, F., et al. Clin. Cancer Res. 7, pp. 958s-965s (2001); Rodriguez, P. C., et al. J. Biol. Chem. 277, pp. 21123-21129 (2002). However the significance of these changes was confirmed using normal human T-cells.

To determine whether the H. pylori sonicate was cytotoxic to the cells or induced apoptosis, two different tests were done. A JAM assay to test cytotoxicity failed to demonstrate any significant reduction in the radioactivity of labeled Jurkat cells, indicating that there were not significant DNA damage of the cells cultured with the H. pylori sonicate (Table I). Furthermore, staining of Jurkat cells with Annexin V after 24 h of culture with the H. pylori sonicate, showed less than 5% apoptotic or necrotic cells (Table II).

Changes in T-cell Signal Transduction Induced by H. pylori:

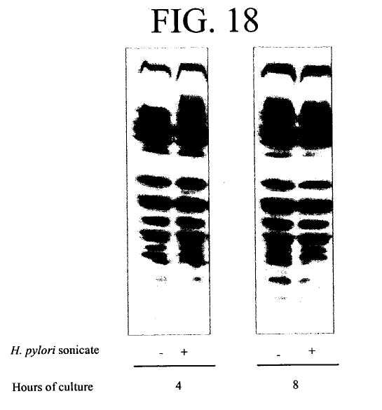

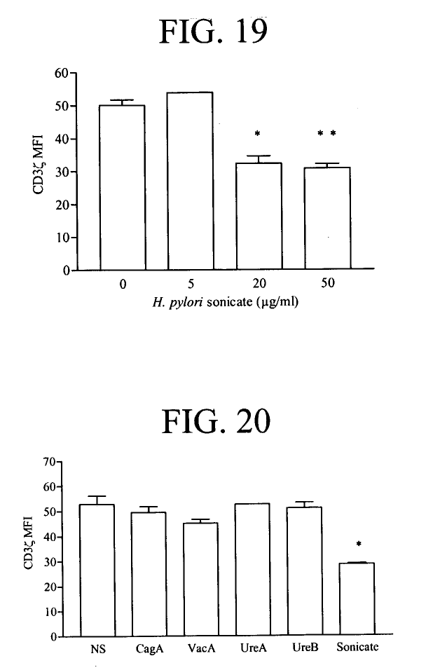

To further explore possible mechanisms leading to a decreased T-cell response induced by the H. pylori sonicate, various aspects of T-cell signal transduction were studied. H. pylori sonicate did not impair the early stages of T-cell signal transduction as determined by Ca⁺⁺ flux (data not shown), nor did it cause alterations in the pattern of tyrosine kinase phosphorylation of Jurkat cells even at high concentrations of the H. pylori sonicate (50 μg/ml) (FIG. 18). The expression of the CD3ζ chain, the main signaling element of the T-cell antigen receptor, were then tested. The H. pylori sonicate down-regulated the expression of the CD3ζ chain (FIG. 19), which paralleled the decrease in T-cell proliferation. Several H. pylori proteins implicated in the pathogenesis of the infection, have also been shown to alter the immune response in vitro. Harris, P. R., et al. J. Infect. Dis. 178, pp. 1516-1520 (1998); Zhang, Q. B., et al. Gut 38, pp. 841-845 (1996). Therefore some of these H. pylori proteins were tested to determine whether they could reduce CD3ζ expression. The recombinant proteins CagA, VacA, urease A (UreA), urease B (UreB) were titrated up to 10 μg/ml, a concentration where they caused cell death within 24 h as determined by trypan blue exclusion. CD3ζ expression did not change when T-cells were cultured with recombinant H. pylori proteins at concentrations known to impair cellular functions. Harris, P. R., et al. J. Infect. Dis. 178, pp. 1516-1520 (1998); Pai, R., et al. Biochem. Biophys. Res. Commun. 262, pp. 245-250 (1999); Rudnicka, W., et al. J. Physiol Pharmacol. 49, pp. 111-119 (1998); Tanahashi, T., et al. Infect. Immun. 68, pp. 664-671 (2000). As shown in FIG. 20, only VacA induced a small, but non-significant decrease in CD3ζ as compared to the H. pylori sonicate. Furthermore, various combinations of these proteins or LPS (derived from E. coli) also failed to decrease the expression of CD3ζ chain (data not shown). Similar to Jurkat cells, human T-cells cultured with purified recombinant H. pylori antigens only showed a significant decrease in the expression of the CD3ζ with the whole H. pylori sonicate (data not shown).

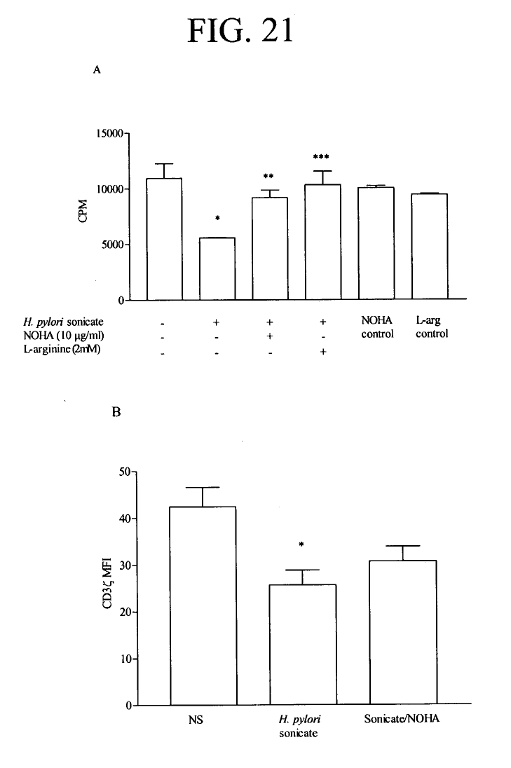

The present inventors have recently shown that production of arginase by macrophages can deplete L-arginine from the microenvironment and induce the loss of CD3ζ in Jurkat and normal T lymphocytes. This process can be prevented by the addition of L-arginine or arginase inhibitors. As shown in FIG. 21A, the addition of the arginase inhibitor NOHA (10 μg/ml) not only prevented the decrease in Jurkat cell proliferation, but also partially inhibited the drop in CD3ζ chain expression (FIGS. 21A and 21B), although the difference in the latter was not found to be statistically significant. The incomplete recovery of CD3ζ chain might be explained by the fact that NOHA appears to only partially inhibit H. pylori arginase (McGee, D. J., manuscript in preparation). The addition of excess L-arginine (2 mM) to Jurkat cells cultured with the H. pylori sonicate also prevented a decrease in cell proliferation. Control Jurkat cells cultured with equivalent concentrations of L-arginine and NOHA did not show a significant variation in either the proliferative capacity or in the expression of CD3ζ.

H. pylori Arginase Reduces CD3ζ Expression and Proliferation of T-cells:

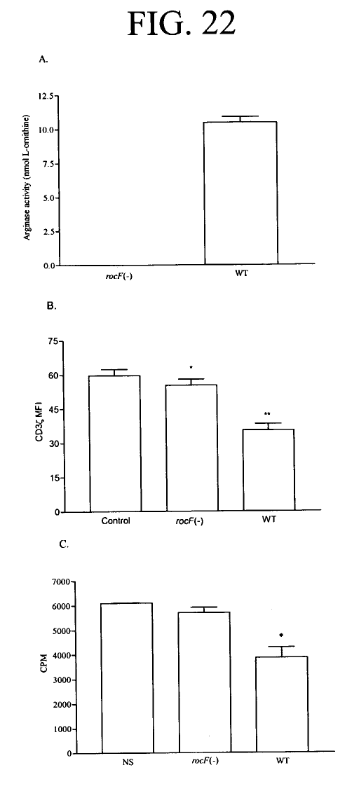

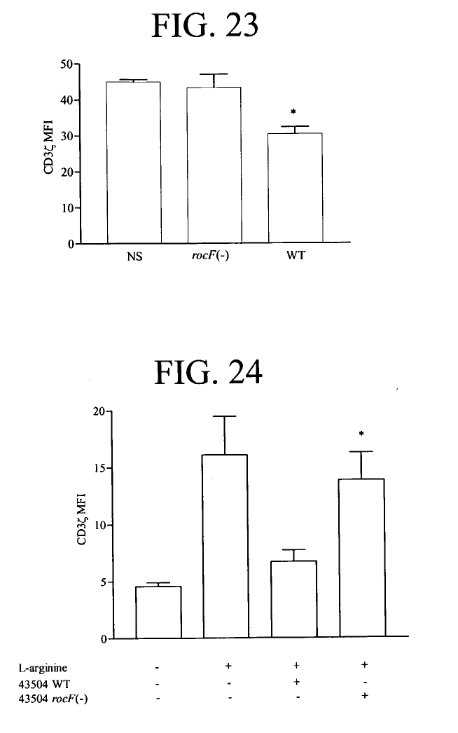

Isogenic H. pylori mutants for arginase were developed from the H. pylori strain ATCC 43504. The arginase gene rocF was inactivated by the insertion of a kanamycin resistance gene. McGee, D. J., et al. J. Bacteriol. 181, pp. 7314-7322 (1999). Arginase activity was measured from sonicates of both the ATCC 43504 wild type and its rocF(−) isogenic arginase mutant. As shown in FIG. 22A, the rocF mutant did not have detectable arginase activity compared to the parental WT 43504 strain, as measured by the ability to metabolize L-arginine to L-ornithine. This result was confirmed by showing that the WT H. pylori bacteria could reduce the L-arginine concentration in the culture medium, while the rocF(−) H. pylori could not (Table III). Culture of Jurkat cells with the WT H. pylori sonicates reduced CD3ζ chain expression and the proliferation of Jurkat cells (FIGS. 22B and 22C). In contrast, the culture of Jurkat cells with the rocF(−) H. pylori sonicate did not alter the expression of the T-cell receptor CD3ζ chain. A similar decrease in the expression of the CD3ζ was induced when live bacteria were co-cultured with Jurkat cells using a Transwell system that kept bacteria separated from the cells (FIG. 23), indicating that bacteria-T-cell contact is not required for the for the induction of these molecular changes.

H. pylori Arginase Reduces the Expression of the CD3ζ Chain in Activated Human T Lymphocytes:

The effect of H. pylori was also tested on freshly isolated normal human T lymphocytes. Activation and culture of T lymphocytes in the absence of L-arginine also causes the loss of CD3ζ, Taheri, F., et al. Clin. Cancer Res. 7, pp. 958s-965s (2001), however, the mechanisms are different from Jurkat cells. In normal T-cells, antigen stimulation causes the internalization of the TCR and the degradation of CD3ζ. Valitutti, S., et al. J. Exp. Med. 185, pp. 1859-1864 (1997). This is followed by synthesis of new CD3ζ protein and the re-expression of the TCR within 48-72 hours. T-cells stimulated in media depleted of L-arginine have a prolonged decrease in CD3ζ that is only reversed by the replenishment of the amino acid. The loss in CD3ζ is not observed when resting cells are cultured in the absence of L-arginine. As shown in FIG. 24 stimulated T-cells cultured in the absence of L-arginine for 48 hours, had a low expression of CD3ζ, while those cultured in medium with L-arginine (400 μM) recovered CD3ζ expression (had undergone the normal cycle of internalization and re-expression of the TCR). In contrast, stimulated T-cells co-cultured with the sonicate from the wild type H. pylori 43504 had a low CD3ζ expression, while T-cells cultured with the sonicate from the arginase mutant had re-expressed CD3ζ (FIG. 24). Therefore, co-culture of stimulated T-cells with WT H. pylori strains that produce arginase has the same deleterious effect on CD3ζ expression as that seen in cells cultured in the absence of L-arginine.

### Discussion of Results

H. pylori infection induces an inflammatory response characterized by infiltrating polymorphonuclear leukocytes, macrophages and lymphocytes, and the production of several inflammatory cytokines including TNF-60 , IFN-γ and IL8. Tanahashi, T., et al. Infect. Immun. 68, pp. 664-671 (2000); Bauditz, J., et al. Clin. Exp. Immunol. 117, pp. 316-323 (1999); Beales, I. L., et al. Cytokine 9, pp. 514-520 (1997); Sharma, S. A., et al. J. Immunol. 160, pp. 2401-2407 (1998); Yamada, H., et al. Biochem. Pharmacol. 61, pp. 1595-1604 (2001). However, this strong immune response appears to confer little or no protection against H. pylori infection. In vitro models show that virulent strains of H. pylori (carrying the PAI) can impair phagocytosis by delaying actin rearrangement. Allen, L. A., et al. J. Exp. Med. 191, pp. 115-128 (2000). Once phagocytosed, these strains of H. pylori cause the fusion of phagosomes into megasomes, decreasing the killing ability of macrophages. Allen, L. A., et al. J. Exp. Med. 191, pp. 115-128 (2000). In doing so, H. pylori not only delays its own phagocytosis, but also that of other particles and bacteria. Ramarao, N., et al. Infect. Immun. 69, pp. 2604-2611 (2001). However, little is known on how H. pylori affects T-cells.

Several reports have also shown that co-culture of T-cells with H. pylori or H. pylori-derived products decreases their response to mitogens. Knipp, U., et al. Med. Microbiol. Immunol.(Berl) 182, pp. 63-76 (1993); Knipp, U., et al. FEMS Immunol. Med. Microbiol. 8, pp. 157-166 (1994); Knipp, U., et al. Infect. Immun. 64, pp. 3491-3496 (1996). The data in this example confirms this effect with an H. pylori sonicate that reduced the proliferation of Jurkat cells and freshly isolated T-cells in a dose- and time-dependent manner, an effect that was not reversible by time in culture. Jurkat cells were used as an indicator of arginase effects because in the absence of L-arginine they undergo a reduction in proliferation and CD3ζ expression similar to anergic cells. Taheri, F., et al. Clin. Cancer Res. 7, pp. 958s-965s (2001); Rodriguez, P. C., et al. J. Biol. Chem. 277, pp. 21123-21129 (2002). However, most of the findings were also tested in normal T-cells. The reduced CD3ζ expression and the diminished proliferation did not appear to be mediated by apoptosis as has previously been reported, Wang, J., et al. J. Immunol. 167, pp. 926-934 (2001), since only a small amount of apoptotic cells were observed (<5%) in this example. It is possible that the activation signals used to stimulate T-cells (anti-CD3 plus anti-CD28) are a strong enough anti-apoptotic signal to prevent programmed cell death induced by the H. pylori extract. Instead, a decreased expression of CD3ζ chain, the principal signal transduction protein in the T-cell receptor was found. T-cell activation is initiated by the binding of antigens to the αβ chains of the TCR, which triggers the phosphorylation of the CD3ζ chain. This protein has three sequences known as immuno-receptor tyrosine-based activation motif (ITAM), which in turn phosphorylate other tyrosine kinases including ZAP-70 and eventually lead to T-cell activation. A decreased expression of CD3ζ has been demonstrated in various chronic infections such as leprosy, tuberculosis and AIDS, Geertsma, M. F., et al. J. Infect. Dis. 180, pp. 649-658 (1999); Seitzer, U., et al. Immunology 104, pp. 269-277 (2001); Trimble, L. A., et al. J. Virol. 74, pp. 7320-7330 (2000); Zea, A. H., et al. Infect. Immun. 66, pp. 499-504 (1998), and appears to partially explain the T-cell anergy that characterizes some of these diseases. Kurt, R. A., et al. Int. J. Cancer 78, pp. 16-20 (1998). Changes in other signal transduction molecules have also been described including a decreased Jak-3 tyrosine kinase and abnormal expression or function of NF-κβ p65 nuclear transcription factor, Kurt, R. A., et al. Int. J. Cancer 78, pp. 16-20 (1998), although these additional changes have not been studied in H. pylori infections

The mechanisms by which L-arginine depletion causes alterations in T-cell signal transduction in these infectious diseases is still unclear. The inventors have previously shown that Jurkat cells or antigen stimulated T-cells cultured in medium without L-arginine undergo a rapid reduction of CD3ζ, have a decreased proliferation and a diminished production of cytokines such as IFNγ. Taheri, F., et al. Clin. Cancer Res. 7, pp. 958s-965s (2001). The diminished expression of CD3ζ chain appears to be caused by a decrease in CD3ζ-chain mRNA stability. Rodriguez, P. C., et al. J. Biol. Chem. 277, pp. 21123-21129 (2002). L-arginine is essential for H. pylori survival. L-arginine is metabolized by arginase into L-ornithine and urea, providing a substrate for urease to synthesize ammonia and carbon dioxide, thereby protecting the bacteria from the harsh acidic environment of the stomach. H. pylori arginase activity was initially described by Mendz and Hazell, Mendz, et al. Microbiology 142 ( Pt 10), pp. 2959-2967 (1996), in experiments using L-arginine as the sole carbon source and measuring the accumulation of L-ornithine and urea by nuclear magnetic resonance (¹H-NMR). They suggested that H. pylori arginase activity was associated with the inner cell membrane and that its activity was dependent on cobalt as a cofactor. Mendz, et al. Microbiology 142 ( Pt 10), pp. 2959-2967 (1996). The latter characteristic differentiates H. pylori arginase from arginase produced by macrophages, which uses Mn⁺⁺ as its main cofactor. Carvajal, N., et al. Comp. Biochem. Physiol. B. Biochem. Mol. Biol. 112, pp. 153-159 (1995). Gobert et al., Gobert, A. P., et al. J. Immunol. 168, pp. 4692-4700 (2002), recently reported that H. pylori also induces arginase II in macrophages, a process that was linked to an increased macrophage apoptosis. In addition, H. pylori arginase can also impair the bactericidal activity of macrophages by inhibiting the production of nitric oxide via L-arginine depletion. Gobert, A. P., et al. Proc. Natl. Acad. Sci.U.S.A 98, pp. 13844-13849 (2001). Our data suggest that arginase activity from H. pylori alters the expression of CD3ζ and T-cell proliferation by decreasing L-arginine availability. Therefore, the enzymatic pathway used by H. pylori for the production of urea needed for its survival in the gastric environment could also serve as a mechanism for impairing macrophage and T-cell responses. It is possible that this mechanism may in part explain the lack of protective effect of the immune response and the chronicity of this infection.

It is also possible that, as shown by Gobert et al., Gobert, A. P., et al. J. Immunol. 168, pp. 4692-4700 (2002), H. pylori antigens can translocate into the gastric mucosa and induce the production arginase by host macrophages, which could also limit the availability of L-arginine and induce T-cell dysfunction. Preliminary data suggests that these changes occur in patients with H. pylori infection (Zabaleta et al, manuscript in preparation). However, the impact of this mechanism on the development and the outcome of H. pylori infection is yet to be determined in vivo.

### Tables I-III of Example VI

### Example VII

This Example Demonstrates That the Effects of Arginase Inhibitors on Arginase Produced by Tumor Cells.

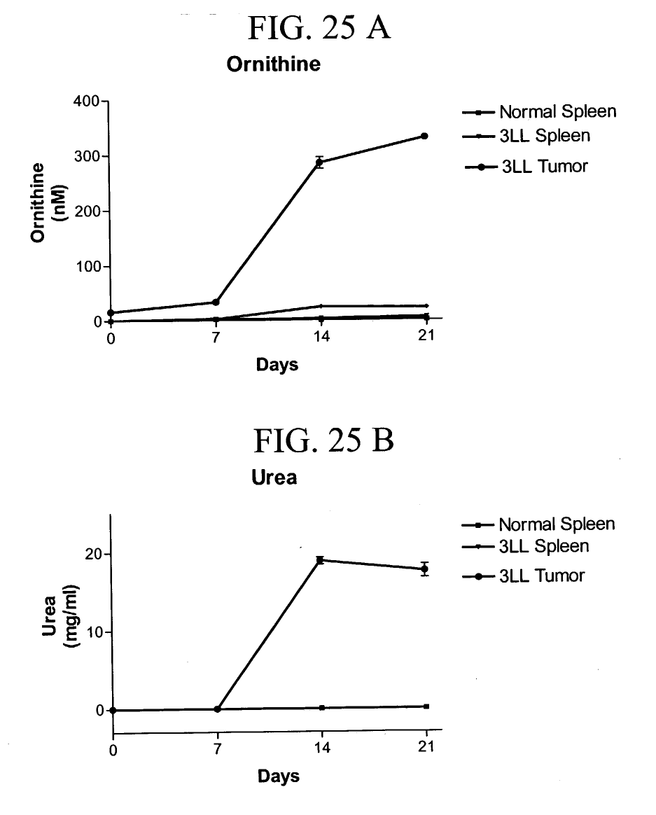

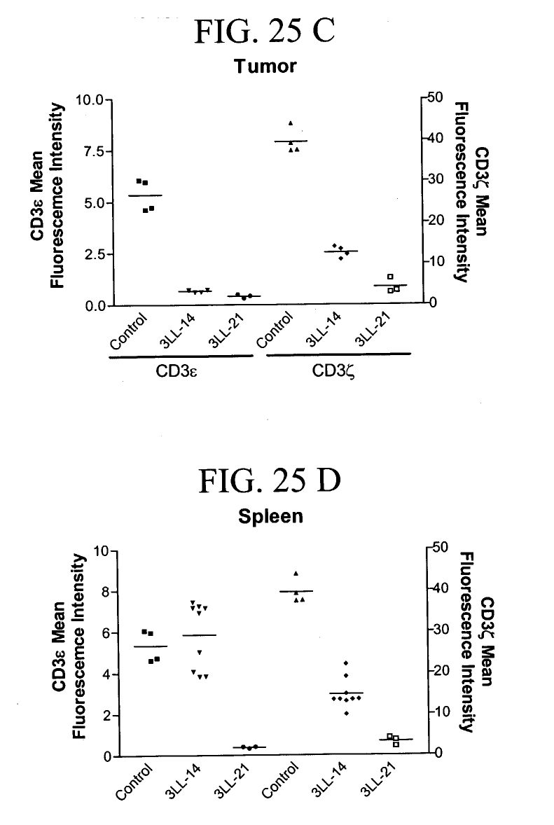

Arginase production in murine tumors. C57Bl/6 mice were injected subcutaneously with one million 3LL lung carcinoma cell lines on the flank. Mice were sacrificed on day 7, 14 and 21 after tumor implantation. Tumors and spleens were removed and arginase activity was measured by testing the conversion of arginine to ornithine and urea. The data demonstrates arginase activity in the tumor by day 7 with a marked increase by day 14 and 21 (FIGS. 25 A and B). The expression of z chain was measured in T cells infiltrating the tumor and in T cells from the spleen (FIGS. 25 C and D). T cells from both sites show a decreased expression of z chain.

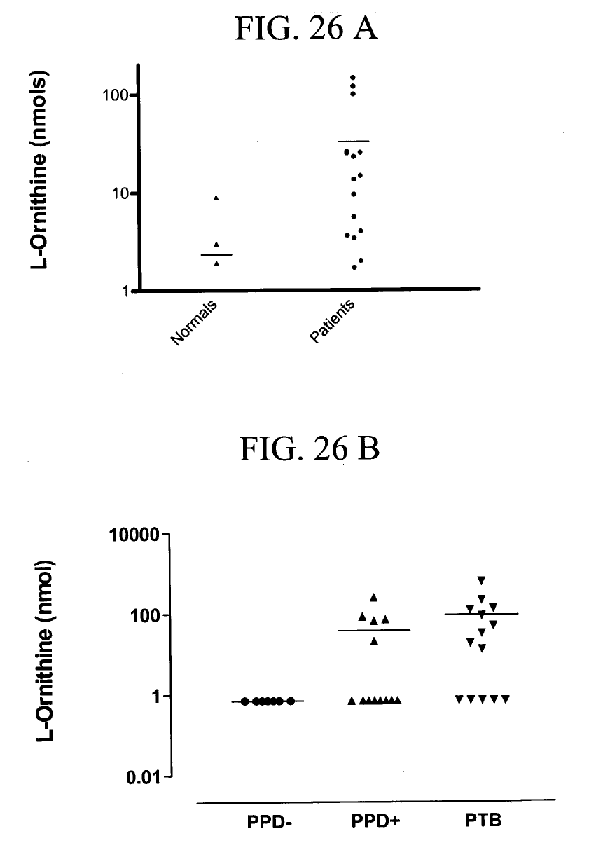

The anti-tumor effect of arginase inhibitor Nor-NOHA was also measured on these tumor cells. C57Bl/6 mice were injected with one million 3LL lung carcinoma cells on the flank. In the contralateral flank mice received different concentrations of nor-NOHA subcutaneously (20 or 40 mgs/kg). Control mice received normal saline solution. Tumors were measured and the tumor volume was calculated in cubic millimiters. The results, shown in the table below and FIGS. 26 A and B, show a significant arrest of tumor growth in mice receiving nor-NOHA. The highest dose tested (40 mgs/kg) was most effective. Materials and methods for this example were similar to those described in the above examples.

Similar results in murine tumors in which arginase production has been tested by us include 3LL lung carcinoma and MCA-38 colon carcinoma. Additionally, in humans, increased arginase production has been demonstrated in renal cells carcinoma where arginase was increased in peripheral blood mononuclear cells and in prostate cancer where arginase is produced by the tumor itself.

The present methods can be carried out by performing any of the steps described herein, either alone or in various combinations. Additionally, one skilled in the art will realize that the present invention also encompasses variations of the present methods that specifically exclude one or more of the steps described above.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than” and the like include the number recited and refer to ranges which can be subsequently broken down into subranges as discussed above. In the same manner, all ratios disclosed herein also include all subratios falling within the broader ratio.

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the claimed invention.

All references disclosed herein are specifically incorporated herein by reference thereto.

While preferred embodiments have been illustrated and described, it should be understood that changes and modifications can be made therein in accordance with ordinary skill in the art without departing from the invention in its broader aspects as defined in the following claims.

Specifically and additionally, the following publication is incorporated into this disclosure by reference:

Bernard, A. C., Mistry, S. K., Morris, S. M., Jr., O'Brien, W. E., Tsuei, B. J., Maley, M. E., Shirley, L. A., Kearney, P. A., Boulanger, B. R., Ochoa, J. B. 2001 “Alterations in arginine metabolic enzymes in trauma. Shock, Vol 15(3):215 (March 2001).”

The following articles and disclosures, enclosed herewith, hereby explicitly form part of the present application:

“The regulation of Signal Transduction Proteins in Immune Cells by Micronutrients including L-arginine”;

Memorandum: “Alterations in Arginine Metabolism and Up Regulation of Arginase in Gene Expression After Trauma”;

“L-Arginine Regulates the Expression of the T-Cell Receptor ζ Chain (CD3ζ) in Jurkat Cells”;

“Arginase I Expression and Activity in Human Mononuclear Cells After Injury”, Annals of Surgery, Vol. 233, No. 3, pp. 393-399;

“Arginase I Expression and Activity in Human Mononuclear Cells After Injury”;

“Trauma Increases Extra-hepatic Arginase Activity”; and

“Leprosy/Tuberculosis Presentation.”

### Reference List

1. Albina, J. E., M. D. Caldwell, W. L. Henry, Jr., and C. D. Mills. 1989. Regulation of macrophage functions by L-arginine. J Exp Med 169:1021-1029.

2. Mills, C. D. 2001. Macrophage arginine metabolism to ornithine/urea or nitric oxide/citrulline: a life or death issue. Crit Rev Immunol 21:399-425.

3. Wu, G. and S. M. Morris, Jr. 1998. Arginine metabolism: nitric oxide and beyond. Biochem.J 336 ( Pt 1):1-17.

4. Amber, I. J., J. B. Hibbs, Jr., C. J. Parker, B. B. Johnson, R. R. Taintor, and Z. Vavrin. 1991. Activated macrophage conditioned medium: identification of the soluble factors inducing cytotoxicity and the L-arginine dependent effector mechanism. J.Leukoc.Biol. 49:610-620.

5. Hibbs, J. B., Jr., R. R. Taintor, and Z. Vavrin. 1987. Macrophage cytotoxicity: role for L-arginine deiminase and imino nitrogen oxidation to nitrite. Science 235:473-476.

6. Chang, C. I., J. C. Liao, and L. Kuo. 2001. Macrophage arginase promotes tumor cell growth and suppresses nitric oxide-mediated tumor cytotoxicity. Cancer Res. 61:1100-1106.

7. Hesse, M., M. Modolell, A. C. La Flamme, M. Schito, J. M. Fuentes, A. W. Cheever, E. J. Pearce, and T. A. Wynn. 2001. Differential regulation of nitric oxide synthase-2 and arginase-1 by type 1/type 2 cytokines in vivo: granulomatous pathology is shaped by the pattern of L-arginine metabolism. J Immunol 167:6533-6544.

8. Munder, M., K. Eichmann, J. M. Moran, F. Centeno, G. Soler, and M. Modolell. 1999. Th1/Th2-regulated expression of arginase isoforms in murine macrophages and dendritic cells. J Immunol 163:3771-3777.

9. Munder, M., K. Eichmann, and M. Modolell. 1998. Alternative metabolic states in murine macrophages reflected by the nitric oxide synthase/arginase balance: competitive regulation by CD4+ T-cells correlates with Th1/Th2 phenotype. J Immunol 160:5347-5354.

10. Mori, M. and T. Gotoh. 2000. Regulation of nitric oxide production by arginine metabolic enzymes. Biochem.Biophys.Res Commun. 275:715-719.

11. Rutschman, R., R. Lang, M. Hesse, J. N. Ihle, T. A. Wynn, and P. J. Murray. 2001. Cutting edge: Stat6-dependent substrate depletion regulates nitric oxide production. J Immunol 166:2173-2177.

12. Louis, C. A., V. Mody, W. L. Henry, Jr., J. S. Reichner, and J. E. Albina. 1999. Regulation of arginase isoforms I and II by IL-4 in cultured murine peritoneal macrophages. Am.J Physiol 276:R237-R242.

13. Roth, E., R. Steininger, S. Winkler, F. Langle, T. Grunberger, R. Fugger, and F. Muhlbacher. 1994. L-Arginine deficiency after liver transplantation as an effect of arginase efflux from the graft. Influence on nitric oxide metabolism. Transplantation 57:665-669.

14. Langle, F., R. Steininger, R. Roth, S. Winkler, H. Andel, S. Acimovic, R. Fugger, and F. Muhlbacher. 1995. L-arginine deficiency and hemodynamic changes as a result of arginase efflux following orthotopic liver transplantation. Transplant.Proc. 27:2872-2873.

15. Bernard, A. C., S. K. Mistry, S. M. Morris, Jr., W. E. O'Brien, B. J. Tsuei, M. E. Maley, L. A. Shirley, P. A. Kearney, B. R. Boulanger, and J. B. Ochoa. 2001. Alterations in arginine metabolic enzymes in trauma. Shock 15:215-219.

16. Schaffer, M. and A. Barbul. 1998. Lymphocyte function in wound healing and following injury. Br.J.Surg. 85:444-460.

17. Barbul, A., H. L. Wasserkrug, N. Yoshimura, R. Tao, and G. Efron. 1984. High arginine levels in intravenous hyperalimentation abrogate post-traumatic immune suppression. J.Surg.Res. 36:620-624.

18. Barbul, A. 1990. Arginine and immune function. Nutrition 6:53-58.

19. Moore, F. A., E. E. Moore, K. A. Kudsk, R. O. Brown, R. H. Bower, M. J. Koruda, C. C. Baker, and A. Barbul. 1994. Clinical benefits of an immune-enhancing diet for early postinjury enteral feeding. J.Trauma 37:607-615.

20. Daly, J. M., J. Reynolds, A. Thom, L. Kinsley, M. Dietrick-Gallagher, J. Shou, and B. Ruggieri. 1988. Immune and metabolic effects of arginine in the surgical patient. Ann.Surg. 208:512-523.

21. Taheri, F., J. B. Ochoa, Z. Faghiri, K. Culotta, H. J. Park, M. S. Lan, A. H. Zea, and A. C. Ochoa. 2001. L-Arginine regulates the expression of the T-cell receptor zeta chain (CD3zeta) in Jurkat cells. Clin Cancer Res 7:958s-965s.

22. Irving, B. A. and A. Weiss. 1991. The cytoplasmic domain of the T-cell receptor zeta chain is sufficient to couple to receptor-associated signal transduction pathways. Cell 64:891-901.

23. Rodriguez, P. C., A. H. Zea, K. S. Culotta, J. Zabaleta, J. B. Ochoa, and A. C. Ochoa. 2002. Regulation of T-cell receptor CD3 zeta chain expression by L-arginine. J Biol Chem 277:21123-21129.

24. O'Quinn, P. R., D. A. Knabe, and G. Wu. 2002. Arginine catabolism in lactating porcine mammary tissue. J Anim Sci. 80:467-474.

25. Kono, K., F. Salazar-Onfray, M. Petersson, J. Hansson, G. Masucci, K. Wasserman, T. Nakazawa, P. Anderson, and R. Kiessling. 1996. Hydrogen peroxide secreted by tumor-derived macrophages down-modulates signal-transducing zeta molecules and inhibits tumor-specific T-cell- and natural killer cell-mediated cytotoxicity. Eur J Immunol 26:1308-1313.

26. Otsuji, M., Y. Kimura, T. Aoe, Y. Okamoto, and T. Saito. 1996. Oxidative stress by tumor-derived macrophages suppresses the expression of CD3 zeta chain of T-cell receptor complex and antigen-specific T-cell responses. Proc.Natl.Acad.Sci.U.S.A 93:13119-13124.

27. Schmielau, J. and O. J. Finn. 2001. Activated granulocytes and granulocyte-derived hydrogen peroxide are the underlying mechanism of suppression of t-cell function in advanced cancer patients. Cancer Res. 61:4756-4760.

28. Closs, E. I., F. Z. Basha, A. Habermeier, and U. Forstermann. 1997. Interference of L-arginine analogues with L-arginine transport mediated by the y+ carrier hCAT-2B. Nitric.Oxide. 1:65-73.

29. Ochoa, J. B., A. O. Udekwu, T. R. Billiar, R. D. Curran, F. B. Cerra, R. L. Simmons, and A. B. Peitzman. 1991. Nitrogen oxide levels in patients after trauma and during sepsis. Ann.Surg. 214:621-626.

30. Ochoa, J. B., A. C. Bernard, S. K. Mistry, S. M. Morris, Jr., P. L. Figert, M. E. Maley, B. J. Tsuei, B. R. Boulanger, and P. A. Kearney. 2000. Trauma increases extrahepatic arginase activity. Surgery 127:419-426.

31. Correa, M. R., A. C. Ochoa, P. Ghosh, H. Mizoguchi, L. Harvey, and D. L. Longo. 1997. Sequential development of structural and functional alterations in T-cells from tumor-bearing mice. J Immunol 158:5292-5296.

32. Mizoguchi, H., J. J. O'Shea, D. L. Longo, C. M. Loeffler, D. W. McVicar, and A. C. Ochoa. 1992. Alterations in signal transduction molecules in T lymphocytes from tumor-bearing mice. Science 258:1795-1798.

33. Zea, A. H., M. T. Ochoa, P. Ghosh, D. L. Longo, W. G. Alvord, L. Valderrama, R. Falabella, L. K. Harvey, N. Saravia, L. H. Moreno, and A. C. Ochoa. 1998. Changes in expression of signal transduction proteins in T lymphocytes of patients with leprosy. Infect.Immun. 66:499-504.

34. Spellberg, B. and J. E. Edwards, Jr. 2001. Type 1/Type 2 immunity in infectious diseases. Clin.Infect.Dis. 32:76-102.

35. Iniesta, V., L. Carlos Gomez-Nieto, I. Molano, A. Mohedano, J. Carcelen, C. Miron, C. Alonso, and I. Corraliza. 2002. Arginase I induction in macrophages, triggered by Th2-type cytokines, supports the growth of intracellular Leishmania parasites. Parasite Immunol 24:113-118.

36. Taylor-Robinson, A. 2001. Th1/Th2-regulated arginase availability modulates Leishmania infection. Trends Parasitol. 17:262.

37. Que, L. G., S. E. George, T. Gotoh, M. Mori, and Y. C. Huang. 2002. Effects of arginase isoforms on NO Production by nNOS. Nitric.Oxide. 6:1-8.

38. de Jonge, W. J., M. M. Hallemeesch, K. L. Kwikkers, J. M. Ruijter, C. Gier-de Vries, M. A. van Roon, A. J. Meijer, B. Marescau, P. P. de Deyn, N. E. Deutz, and W. H. Lamers. 2002. Overexpression of arginase I in enterocytes of transgenic mice elicits a selective arginine deficiency and affects skin, muscle, and lymphoid development. Am.J Clin.Nutr. 76:128-140.

39. White, M. F. 1985. The transport of cationic amino acids across the plasma membrane of mammalian cells. Biochim.Biophys.Acta 822:355-374.

40. Closs, E. I. 2002. Expression, regulation and function of carrier proteins for cationic amino acids. Curr.Opin.Nephrol.Hypertens. 11:99-107.

41. Finke, J. H., A. H. Zea, J. Stanley, D. L. Longo, H. Mizoguchi, R. R. Tubbs, R. H. Wiltrout, J. J. O'Shea, S. Kudoh, E. Klein, and . 1993. Loss of T-cell receptor zeta chain and p561ck in T-cells infiltrating human renal cell carcinoma. Cancer Res. 53:5613-5616.

42. Fafournoux, P., A. Bruhat, and C. Jousse. 2000. Amino acid regulation of gene expression. Biochem J 351:1-12.

43. Munn, D. H., E. Shafizadeh, J. T. Attwood, I. Bondarev, A. Pashine, and A. L. Mellor. 1999. Inhibition of T-cell proliferation by macrophage tryptophan catabolism. J.Exp.Med. 189:1363-1372.

44. Lee, G. K., H. J. Park, M. Macleod, P. Chandler, D. H. Munn, and A. L. Mellor. 2002. Tryptophan deprivation sensitizes activated T-cells to apoptosis prior to cell division. Immunology 107:452-460.

45. Mellor, A. L. and D. H. Munn. 1999. Tryptophan catabolism and T-cell tolerance: immunosuppression by starvation? Immunol.Today 20:469-473.

46. Bruhat, A., C. Jousse, X. Z. Wang, D. Ron, M. Ferrara, and P. Fafournoux. 1997. Amino acid limitation induces expression of CHOP, a CCAAT/enhancer binding protein-related gene, at both transcriptional and post- transcriptional levels. J Biol Chem 272:17588-17593.

47. Bruhat, A., C. Jousse, and P. Fafournoux. 1999. Amino acid limitation regulates gene expression. Proc.Nutr.Soc. 58:625-632.

48. Wu, C. W., C. W. Chi, E. C. Lin, W. Y. Lui, F. K. P'eng, and S. R. Wang. 1994. Serum arginase level in patients with gastric cancer. J Clin.Gastroenterol. 18:84-85.

49. Wu, C. W., W. W. Chung, C. W. Chi, H. L. Kao, W. Y. Lui, F. K. P'eng, and S. R. Wang. 1996. Immunohistochemical study of arginase in cancer of the stomach. Virchows Arch. 428:325-331.

50. Selamnia, M., C. Mayeur, V. Robert, and F. Blachier. 1998. Alpha-difluoromethylornithine (DFMO) as a potent arginase activity inhibitor in human colon carcinoma cells. Biochem.Pharmacol. 55:1241-1245.

51. Suer, G. S., Y. Yoruk, E. Cakir, F. Yorulmaz, and S. Gulen. 1999. Arginase and ornithine, as markers in human non-small cell lung carcinoma. Cancer Biochem Biophys. 17:125-131.

52. Singh, R., S. Pervin, A. Karimi, S. Cederbaum, and G. Chaudhuri. 2000. Arginase activity in human breast cancer cell lines: N(omega)-hydroxy-L-arginine selectively inhibits cell proliferation and induces apoptosis in MDA-MB-468 cells. Cancer Res. 60:3305-3312.

53. Wei, L. H., G. Wu, S. M. Morris, Jr., and L. J. Ignarro. 2001. Elevated arginase I expression in rat aortic smooth muscle cells increases cell proliferation. Proc.Natl.Acad.Sci.U.S.A 98:9260-9264.

54. da Silva, E. R., T. M. Castilho, F. C. Pioker, Tomich de Paula Silva CH, and L. M. Floeter-Winter. 2002. Genomic organisation and transcription characterisation of the gene encoding Leishmania (Leishmania) amazonensis arginase and its protein structure prediction. Int.J.Parasitol. 32:727-737.

55. Gobert, A. P., D. J. McGee, M. Akhtar, G. L. Mendz, J. C. Newton, Y. Cheng, H. L. Mobley, and K. T. Wilson. 2001. Helicobacter pylori arginase inhibits nitric oxide production by eukaryotic cells: a strategy for bacterial survival. Proc.Natl.Acad.Sci.U.S.A 98:13844-13849.

56. Mendz, G. L., E. M. Holmes, and R. L. Ferrero. 1998. In situ characterization of Helicobacter pylori arginase. Biochim.Biophys.Acta 1388:465-477.

57. Liossis, S. N., X. Z. Ding, G. J. Dennis, and G. C. Tsokos. 1998. Altered pattern of TCR/CD3-mediated protein-tyrosyl phosphorylation in T-cells from patients with systemic lupus erythematosus. Deficient expression of the T-cell receptor zeta chain. J Clin.Invest 101:1448-1457.

58. Rabinowich, H., T. E. Reichert, Y. Kashii, B. R. Gastman, M. C. Bell, and T. L. Whiteside. 1998. Lymphocyte apoptosis induced by Fas li. J Clin.Invest 101:2579-2588.

59. Schmielau, J., M. A. Nalesnik, and O. J. Finn. 2001. Suppressed T-cell receptor zeta chain expression and cytokine production in pancreatic cancer patients. Clin.Cancer Res. 7:933s-939s.

What is claimed is:

1. A method of treating an arginase I mediated immune suppression in a mammal in need thereof, comprising:
administering an effective amount of an inhibitor of arginase I, an inhibitor of a cationic amino acid transporter Y+ receptor or a liposomal formulation of arginine or an arginine provider to a mammal wherein an immune response in the mammal is increased.

2. The method of claim 1 wherein the mammal is a human.

3. The method of claim 1 wherein the arginase I mediated immune suppression is caused by a chronic infectious disease, autoimmune disease, trauma, leprosy, tuberculosis, liver transplantation, infectious microorganisms such as bacteria or parasites or a cancer.

4. The method of claim 1 wherein the inhibitor of arginase I or the inhibitor of the cationic amino acid transporter Y+ receptor is selected from the group consisting of cycloheximide, NOHA, nor-NOHA, ornithine, lysine, norvaline, adrenergic blocking agents, propanolol, a cytokine, L-mono-methyl-L-arginine (NMMA), a boronic acid based compound, 2(S)-amino-6-boronohexanoic acid (ABH) and S-(2-boronoethyl)-L-cysteine (BEC), and combinations thereof.

5. The method of claim 1 wherein the immune response increased in the mammal comprises increasing stimulated T-cell proliferation, T-cell function or both.

6. The method of claim 1 wherein the increased immune response is determined by measuring arginase I activity, arginase I levels, arginine levels, T-cell function, T-cell proliferation, TCR zeta chain expression after antigen stimulation and combinations thereof.

7. The method of claim 1 wherein the immune response increased in the mammal is a systemic immune response.

8. The method of claim 1 wherein the arginase inhibitor preferentially inhibits arginase I compared to arginase II.

9. The method of claim 5 wherein the inhibitor of arginase, the inhibitor of a cationic amino acid transporter Y+ receptor or the liposomal formulation of arginine or an arginine provider is administered in amount such that the arginine level available to the T-cells of the subject is about 40 μM or greater.

10. A method of treating an arginase mediated immune suppression resulting from a bacterial or viral infection in a mammal in need thereof, comprising:
administering an effective amount of an inhibitor of arginase, an inhibitor of a cationic amino acid transporter Y+ receptor or a liposomal formulation of arginine or an arginine provider to a mammal suffering having a bacterial or viral infection
wherein an immune response in the mammal is increased and further wherein the infection is not a result of leishmaniasis.

11. The method of claim 10 wherein the mammal is a human.

12. The method of claim 10 wherein the arginase I mediated immune suppression is caused by a chronic infectious disease, leprosy, tuberculosis, an infectious microorganisms or a virus.

13. The method of claim 10 wherein the inhibitor of arginase I or the inhibitor of the cationic amino acid transporter Y+ receptor is selected from the group consisting of cycloheximide, NOHA, nor-NOHA, ornithine, lysine, norvaline, adrenergic blocking agents, propanolol, a cytokine, L-mono-methyl-L-arginine (NMMA), a boronic acid based compound, 2(S)-amino-6-boronohexanoic acid (ABH) and S-(2-boronoethyl)-L-cysteine (BEC), and combinations thereof.

14. The method of claim 10 wherein the immune response increased in the mammal comprises increasing stimulated T-cell proliferation, function or both.

15. The method of claim 10 wherein the increased immune response is determined by measuring arginase I activity, arginase I levels, arginine levels, T-cell function, T-cell proliferation, TCR zeta chain expression after antigen stimulation and combinations thereof.

16. The method of claim 10 wherein the immune response increased in the mammal is a systemic immune response.

17. The method of claim 10 wherein the arginase inhibitor preferentially inhibits arginase I compared to arginase II.

18. The method of claim 14 wherein the inhibitor of arginase, the inhibitor of a cationic amino acid transporter Y+ receptor or the liposomal formulation of arginine or an arginine provider is administered in amount such that the arginine level available to the T-cells of the subject is about 40 μM or greater.

19. A method of therapeutically suppressing an immune response in an animal, comprising:
administering an effective amount of arginase I or a stimulator of arginase I to a mammal wherein an immune response in the mammal is suppressed.

20. The method of claim 19 wherein the stimulator of arginase I is a Th2 cytokine, IL-4, IL-10, IL-13, 8-bromo-cAMP, 8-bromo-cAMP plus Lipopolysaccharide 8-bromo-cAMP and interferon-gamma and combinations thereof.
